(19) United States
(12) Patent Application Publication
(10) Pub. No.: US 2004/0235071 A1
(43) Pub. Date: Nov. 25, 2004

(54) Methods and compositions for treating cancer using 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249

(75) Inventors: Eric S. Lightcap, Natick, MA (US); Jeffrey A. Ecsedy, Jamaica Plain, MA (US); John J. Hunter, Oakland, CA (US); Kyle J. MacBeth, Boston, MA (US); Michelle Tighe Nestor, North Woodstock, NH (US)

Correspondence Address: Jean M. Silveri, Millennium Pharmaceuticals, Inc., 40 Landsdowne Street, Cambridge, MA, 02139, US

(73) Assignee: Millennium Pharmaceuticals, Inc.

(21) Appl. No.: 10/737,450

(22) Filed: Dec. 16, 2003

Related U.S. Application Data
(60) Provisional application No. 60/435,108, filed on Dec. 20, 2002.
(60) Provisional application No. 60/436,443, filed on Dec. 23, 2002.
(60) Provisional application No. 60/438,498, filed on Jan. 7, 2003.
(60) Provisional application No. 60/444,370, filed on Jan. 31, 2003.
(60) Provisional application No. 60/446,031, filed on Feb. 6, 2003.
(60) Provisional application No. 60/453,635, filed on Mar. 11, 2003.
(60) Provisional application No. 60/457,199, filed on Mar. 25, 2003.
(60) Provisional application No. 60/462,458, filed on Apr. 10, 2003.
(60) Provisional application No. 60/466,732, filed on Apr. 30, 2003.
(60) Provisional application No. 60/469,184, filed on May 8, 2003.
(60) Provisional application No. 60/471,663, filed on May 19, 2003.
(60) Provisional application No. 60/475,472, filed on Jun. 3, 2003.
(60) Provisional application No. 60/478,150, filed on Jun. 12, 2003.
(60) Provisional application No. 60/480,631, filed on Jun. 23, 2003.
(60) Provisional application No. 60/487,369, filed on Jul. 15, 2003.
(60) Provisional application No. 60/490,866, filed on Jul. 29, 2003.
(60) Provisional application No. 60/499,614, filed on Sep. 2, 2003.
(60) Provisional application No. 60/510,081, filed on Oct. 9, 2003.
(60) Provisional application No. 60/517,742, filed on Nov. 6, 2003.

(51) Int. Cl. G01N 33/574

(52) U.S. Cl. 435/7.23

(57) ABSTRACT

The present invention relates to methods for the diagnosis and treatment of a cancer or cancer. Specifically, the present invention identifies the differential expression of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 and 13249 genes in tissues relating to cancer, relative to their expression in normal, or non-cancer disease states, and/or in response to manipulations relevant to a cancer. The present invention describes methods for the diagnostic evaluation and prognosis of various cancers, and for the identification of subjects exhibiting a predisposition to such conditions. The invention also provides methods for identifying a compound capable of modulating a cancer or cancer. The present invention also provides methods for the identification and therapeutic use of compounds as treatments of cancer.

### RELATED APPLICATIONS

The present application claims the benefit of U.S. Provisional Application Ser. No. 60/435,108, filed on Dec. 20, 2002, of U.S. Provisional Application Ser. No. 60/436,443, filed on Dec. 23, 2002, of U.S. Provisional Application Ser. No. 60/438,498, filed on Jan. 7, 2003, of U.S. Provisional Application Ser. No. 60/444,370, filed on Jan. 31, 2003, of U.S. Provisional Application Ser. No. 60/446,031, filed on Feb. 6, 2003, of U.S. Provisional Application Ser. No. 60/453,635, filed on Mar. 11, 2003, of U.S. Provisional Application Ser. No. 60/457,199, filed on Mar. 25, 2003, of U.S. Provisional Application Ser. No. 60/462,458, filed on Apr. 10, 2003, of U.S. Provisional Application Ser. No. 60/466,732, filed on Apr. 30, 2003, of U.S. Provisional Application Ser. No. 60/469,184, filed on May 8, 2003, of U.S. Provisional Application Ser. No. 60/471,663, filed on May 19, 2003, of U.S. Provisional Application Ser. No. 60/475,472, filed on Jun. 3, 2003, of U.S. Provisional Application Ser. No. 60/478,150, filed on Jun. 12, 2003, of U.S. Provisional Application Ser. No. 60/480,631, filed on Jun. 23, 2003, of U.S. Provisional Application Ser. No. 60/487,369, filed on Jul. 15, 2003, of U.S. Provisional Application Ser. No. 60/490,866, filed on Jul. 29, 2003, of U.S. Provisional Application Ser. No. 60/499,614, filed on Sep. 2, 2003, of U.S. Provisional Application Ser. No. 60/510,081, filed on Oct. 9, 2003 and of U.S. Provisional Application Ser. No. 60/517,742, filed on Nov. 6, 2003. The entire contents of these provisional patent applications are hereby incorporated in their entirety by this reference.

### BACKGROUND OF THE INVENTION

Cancer can be viewed as a breakdown in the communication between tumor cells and their environment, including their normal neighboring cells. Growth-stimulatory and growth-inhibitory signals are routinely exchanged between cells within a tissue. Normally, cells do not divide in the absence of stimulatory signals or in the presence of inhibitory signals. In a cancerous or neoplastic state, a cell acquires the ability to “override” these signals and to proliferate under conditions in which a normal cell would not.

In general, tumor cells must acquire a number of distinct aberrant traits in order to proliferate in an abnormal manner. Reflecting this requirement is the fact that the genomes of certain well-studied tumors carry several different independently altered genes, including activated oncogenes and inactivated tumor suppressor genes. In addition to abnormal cell proliferation, cells must acquire several other traits for tumor progression to occur. For example, early on in tumor progression, cells must evade the host immune system. Further, as tumor mass increases, the tumor must acquire vasculature (e.g. through neo-angiogenesis) to supply nourishment and remove metabolic waste. Additionally, cells must acquire an ability to invade adjacent tissue. In many cases cells ultimately acquire the capacity to metastasize to distant sites.

Angiogenesis is a fundamental process by which new blood vessels are formed, as reviewed, for example, by Folkman and Shing, J. Biol. Chem. 267:10931-10934 (1992). Capillary blood vessels consist of endothelial cells and pericytes. These two cell types carry all of the genetic information to form tubes, branches and whole capillary networks. Specific angiogenic molecules and growth factors can initiate this process. Specific inhibitory molecules can stop it. These molecules with opposing function appear to be continuously acting in concert to maintain a stable microvasculature in which endothelial cell turnover is thousands of days. However, the same endothelial cells can undergo rapid proliferation, i.e. less than five days, during burst of angiogenesis, for example, during wound healing.

Key components of the angiogenic process are the degradation of the basement membrane, the migration and proliferation of capillary endothelial cell (EC) and the formation of three dimensional capillary tubes. The normal vascular turnover is rather low: the doubling time for capillary endothelium is from 50-20,000 days, but it is 2-13 days for tumor capillary endothelium. The current understanding of the sequence of events leading to angiogenesis is that a cytokine capable of stimulating endothelial cell proliferation, such as fibroblast growth factor (FGF), causes release of collagenase or plasminogen activator which, in turn, degrade the basement membrane of the parent venule to facilitate the migration of the endothelial cells. These capillary cells, having sprouted from the parent vessel, proliferate in response to growth factors and angiogenic agents in the surrounding environment to form lumen and eventually new blood vessels.

The development of a vascular blood supply is essential in reproduction, development and wound repair (Folkman, et al., Science 43:1490-1493 (1989)). Under these conditions, angiogenesis is highly regulated, so that it is turned on only as necessary, usually for brief periods of days, then completely inhibited. However, a number of serious diseases are also dominated by persistent unregulated angiogenesis and/or abnormal neovascularization including solid tumor growth and metastasis, psoriasis, endometriosis, Grave's disease, ischemic disease (e.g., atherosclerosis), and chronic inflammatory diseases (e.g., rheumatoid arthritis), and some types of eye disorders, (reviewed by Auerbach, et al., J. Microvasc. Res. 29:401-411 (1985); Folkman, Advances in Cancer Research, eds. Klein and Weinhouse, pp. 175-203 (Academic Press, New York, 1985); Patz, Am. J. Opthalmol. 94:715-743 (1982); and Folkman, et al., Science 221:719-725 (1983)). For example, there are a number of eye diseases, many of which lead to blindness, in which ocular neovascularization occurs in response to the diseased state. These ocular disorders include diabetic retinopathy, macular degeneration, neovascular glaucoma, inflammatory diseases and ocular tumors (e.g., retinoblastoma). There are a number of other eye diseases which are also associated with neovascularization, including retrolental fibroplasia, uveitis, eye diseases associated with choroidal neovascularization and eye diseases which are associated with iris neovascularization.

It is apparent that the complex process of tumor development and growth must involve multiple gene products. It is therefore important to define the role of specific genes involved in tumor development and growth and identify those genes and gene products that can serve as targets for the diagnosis, prevention and treatment of cancers.

In the realm of cancer therapy it often happens that a therapeutic agent that is initially effective for a given patient becomes, overtime, ineffective or less effective for that patient. The very same therapeutic agent may continue to be effective over a long period of time for a different patient. Further, a therapeutic agent that is effective, at least initially, for some patients can be completely ineffective or even harmful for other patients. Accordingly, there exists a need to identify genes and/or gene products that represent prognostic markers with respect to a given therapeutic agent or class of therapeutic agents. It then may be possible to determine which patients will benefit from particular therapeutic regimen and, importantly, determine when, if ever, the therapeutic regime begins to lose its effectiveness for a given patient. The ability to make such predictions would make it possible to discontinue a therapeutic regime that has lost its effectiveness well before its loss of effectiveness becomes apparent by conventional measures

### DESCRIPTION OF THE INVENTION

The present invention provides methods and compositions for the diagnosis and treatment of cancer and/or angiogenic disorders, including but not limited to cancers of the lung, ovary, prostate, breast or colon, or conditions characterized by an increase or decrease in angiogenesis. The polypeptides and nucleic acids of the invention can also be used to treat, prevent, and/or diagnose cancers and neoplastic conditions in addition to the ones described above. As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

In one aspect, the invention provides methods for identifying a compound capable of treating a cancer and/or angiogenic disease. The method includes assaying the ability of the compound to modulate 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 nucleic acid expression or 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 polypeptide activity. In one embodiment, the ability of the compound to modulate 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 nucleic acid expression or 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 polypeptide activity is determined by detecting modulation of tumorigenesis or angiogenesis in a cell.

In another aspect, the invention provides methods for identifying a compound capable of modulating tumorigenesis or angiogenesis. The method includes contacting a cell expressing a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 nucleic acid or polypeptide with a test compound and assaying the ability of the test compound to modulate the expression of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 nucleic acid or the activity of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 polypeptide.

In yet another aspect, the invention features a method for treating a subject having a cancer and/or angiogenic disorder e.g., a cancer and/or angiogenic disorder characterized by aberrant 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 polypeptide activity or aberrant 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 nucleic acid expression. The method includes administering to the subject a therapeutically effective amount of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 modulator (e.g., using a pharmaceutically acceptable formulation or a gene therapy vector). In one embodiment, the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 modulator may be a small molecule, an anti-15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 antibody, a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 polypeptide, an antisense 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 nucleic acid molecule, or a ribozyme.

As used herein, a “tumorigenic disease or disorder” includes a disease or disorder characterized by aberrantly regulated cell growth, proliferation, differentiation, adhesion, or migration, resulting in the production of or tendency to produce tumors. As used herein, a “tumor” includes a normal benign or malignant mass of tissue. Examples of tumorigenic diseases include cancer, e.g., carcinoma, sarcoma, lymphoma or leukemia, examples of which include, but are not limited to, ovarian, lung, breast, endometrial, uterine, hepatic, gastrointestinal, prostate, colorectal, and brain cancer.

As used herein, the term angiogenic disease or disorder” includes a disease or disorder characterized by aberrantly regulated angiogenesis. As used herein, the term “angiogenesis” refers to the process by which new blood vessels, e.g., blood capillaries, vessels, and veins are formed. Key components of the angiogenic process are the degradation of the basement membrane, the migration and proliferation of capillary endothelial cell (EC) and the formation of three-dimensional capillary tubes. New blood vessels can develop from the walls of existing small vessels by the outgrowth of endothelial cells. Angiogenesis is also involved in tumor growth as it provides tumors with the blood supply necessary for tumor cell survival and proliferation (growth). Examples of angiogenic diseases include solid tumor growth and metastasis, psoriasis, endometriosis, Grave's disease, ischemic disease (e.g., atherosclerosis), and chronic inflammatory diseases (e.g., rheumatoid arthritis), and some types of eye disorders, (reviewed by Auerbach, et al., J. Microvasc. Res. 29:401-411 (1985); Folkman, Advances in Cancer Research, eds. Klein and Weinhouse, pp. 175-203 (Academic Press, New York, 1985); Patz, Am. J. Opthalmol. 94:715-743 (1982); and Folkman, et al., Science 221:719-725 (1983)). For example, there are a number of eye diseases, many of which lead to blindness, in which ocular neovascularization occurs in response to the diseased state. These ocular disorders include diabetic retinopathy, macular degeneration, neovascular glaucoma, inflammatory diseases and ocular tumors (e.g., retinoblastoma). There are a number of other eye diseases which are also associated with neovascularization, including retrolental fibroplasia, uveitis, eye diseases associated with choroidal neovascularization and eye diseases which are associated with iris neovascularization.

Examples of cellular proliferative and/or differentiative disorders include cancer, e.g., carcinoma, sarcoma, or metastatic disorders. The molecules of the present invention can act as novel diagnostic targets and therapeutic agents for controlling breast cancer, ovarian cancer, colon cancer, lung cancer, prostatic cancer, squamous carcinoma of the cervix, as well as metastasis of such cancers and the like. A metastatic tumor can arise from a multitude of primary tumor types, including but not limited to those of breast, lung, liver, colon, ovarian origin, and colom-liver. A cellular proliferative disorder can be an endothelial cell disorder. As used herein, an “endothelial cell disorder” includes a disorder characterized by aberrant, unregulated, or unwanted endothelial cell activity, e.g., proliferation, migration, angiogenesis, or vascularization; or aberrant expression of cell surface adhesion molecules or genes associated with angiogenesis, e.g., TIE-2, FLT and FLK. Endothelial cell disorders include tumorigenesis, tumor metastasis, psoriasis, diabetic retinopathy, endometriosis, Grave's disease, ischemic disease (e.g., atherosclerosis), and chronic inflammatory diseases (e.g., rheumatoid arthritis).

Examples of cancers or neoplastic conditions, in addition to the ones described above, include, but are not limited to, a fibrosarcoma, myosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, gastric cancer, esophageal cancer, rectal cancer, pancreatic cancer, ovarian cancer, prostate cancer, uterine cancer, cancer of the head and neck, skin cancer, brain cancer, squamous cell carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular cancer, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, or Kaposi sarcoma.

Examples of cellular proliferative and/or differentiative disorders of the breast include, but are not limited to, proliferative breast disease including, e.g., epithelial hyperplasia, sclerosing adenosis, and small duct papillomas; tumors, e.g., stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma, and miscellaneous malignant neoplasms. Disorders in the male breast include, but are not limited to, gynecomastia and carcinoma.

Examples of cellular proliferative and/or differentiative disorders of the lung include, but are not limited to, bronchogenic carcinoma, including paraneoplastic syndromes, bronchioloalveolar carcinoma, neuroendocrine tumors, such as bronchial carcinoid, miscellaneous tumors, and metastatic tumors; pathologies of the pleura, including inflammatory pleural effusions, noninflammatory pleural effusions, pneumothorax, and pleural tumors, including solitary fibrous tumors (pleural fibroma) and malignant mesothelioma. Preferred examples of lung tumors that can be treated include small cell carcinoma and poorly differentiated small cell carcinoma of the lung.

Examples of cellular proliferative and/or differentiative disorders of the colon include, but are not limited to, non-neoplastic polyps, adenomas, familial syndromes, colorectal carcinogenesis, colorectal carcinoma, and carcinoid tumors. Preferred examples of colon tumors include moderately differentiated tumors.

Examples of cellular proliferative and/or differentiative disorders of the ovary include, but are not limited to, ovarian tumors such as, tumors of coelomic epithelium, serous tumors, mucinous tumors, endometeriod tumors, clear cell adenocarcinoma, cystadenofibroma, brenner tumor, surface epithelial tumors; germ cell tumors such as mature (benign) teratomas, monodermal teratomas, immature malignant teratomas, dysgerminoma, endodermal sinus tumor, choriocarcinoma; sex cord-stomal tumors such as, granulosa-theca cell tumors, thecoma-fibromas, androblastomas, hill cell tumors, and gonadoblastoma; and metastatic tumors such as Krukenberg tumors.

Examples of prostatic cancerous disorders include adenocarcinoma or carcinoma, of the prostate and/or testicular tumors.

Examples of conditions characterized by an increase or decrease in angiogenesis include but are not limited to solid tumor growth and metastasis, psoriasis, endometriosis, Grave's disease, ischemic disease (e.g., atherosclerosis), and chronic inflammatory diseases (e.g., rheumatoid arthritis), and some types of eye disorders

“Treatment”, as used herein, is defined as the application or administration of a therapeutic agent to a patient, or application or administration of a therapeutic agent to an isolated tissue or cell line from a patient, who has a disease or disorder, a symptom of disease or disorder or a predisposition toward a disease or disorder, with the purpose of curing, healing, alleviating, relieving, altering, remedying, ameliorating, improving or affecting the disease or disorder, at least one symptom of disease or disorder or the predisposition toward a disease or disorder. A therapeutic agent includes, but is not limited to, small molecules, peptides, antibodies, ribozymes, gene therapy vectors and antisense oligonucleotides. Representative molecules are described herein.

The present invention is based, at least in part, on the discovery that nucleic acid and protein molecules, (described infra), are differentially expressed in disease states relative to their expression in normal, or non-disease states. The modulators of the molecules of the present invention, identified according to the methods of the invention, can be used to modulate (e.g., inhibit, treat, or prevent) or diagnose a disease, including, but not limited to, a cancer including but not limited to cancers of the lung, ovary, prostate, breast, colon or other disease state characterized by modulation of angiogenesis.

The modulators of the molecules of the present invention can include but are not limited to small organic molecules, peptides, ribozymes, nucleic acid antisense molecules, gene therapy vectors or antibodies.

“Differential expression”, as used herein, includes both quantitative as well as qualitative differences in the temporal and/or tissue expression pattern of a gene. Thus, a differentially expressed gene may have its expression activated or inactivated in normal versus disease conditions. The degree to which expression differs in normal versus disease or control versus experimental states need only be large enough to be visualized via standard characterization techniques, e.g., quantitative PCR, Northern analysis, subtractive hybridization. The expression pattern of a differentially expressed gene may be used as part of a prognostic or diagnostic of a disease, e.g., a cancer including but not limited to cancers of the lung, ovary, prostate, breast, colon or other disease state characterized by modulation of angiogenesis evaluation, or may be used in methods for identifying compounds useful for the treatment of a disease, e.g., a cancer including but not limited to cancers of the lung, ovary, prostate, breast or colon. In addition, a differentially expressed gene involved in a disease may represent a target gene such that modulation of the level of target gene expression or of target gene product activity will act to cure, heal, alleviate, relieve, alter, remedy, ameliorate, improve or affect a disease condition, e.g., a cancer including but not limited to cancers of the lung, ovary, prostate, breast, colon or other disease state characterized by modulation of angiogenesis. Compounds that modulate target gene expression or activity of the target gene product can be used in the treatment of a disease. Although the genes described herein may be differentially expressed with respect to a disease, and/or their products may interact with gene products important to a disease, the genes may also be involved in mechanisms important to additional disease cell processes.

Molecules of the Present Invention

The molecules of the present invention can be characterized as, or have structural features in common with, molecules of the following functional classes, including but not limited to:

Transferases:

MTAP/PNP family of phosphorylases

2-oxo acid dehydrogenases acyltransferase

adenylate-kinase

1-acyl-sn-glycerol-3-phosphate acyltransferase

AIR synthase and relatives

class II aldolase domain

Aminotransferases

AMP-binding enzymes

arginine N-methyltransferase

Arginosuccinate synthase

NAD:arginine ADP-ribosyltransferase

Asparagine synthase

Asp and Glu kinases

ATP:guanido phosphotransferases

ATP synthase

bile acid CoA:amino acid N-acyltransferase

Biopterin-dependent aromatic amino acid hydroxylase

biotin-requiring enzymes

Beta-ketoacyl synthase

biotin-protein ligase

Carbohydrate phosphorylases

carnitate acyltransferase

CDP-alcohol phosphatidyltransferase

choline transferases

CoA ligases

Coenzyme A transferase

Cys/Met metabolism PLP-dependent enzyme

diacylglycerol kinase

Delta-aminolevulinic acid dehydratase

Dihydrodipicolinate synthetase family

Enol-ase

FGGY carbohydrate kinase family

Formyl transferase

fucosyltransferases

Galactose-1-phosphate uridyl transferase

galactosyl-transferases

Phosphoribosylglycinamide synthetase (GARS)

Type 1 glutamine amidotransferases

Type II glutamine amidotransferases

gamma-glutamyltransferase

GHMP kinases

Glutamine synthetase

glycosyl tferases group 2

type 4 glycosyl transferases

Glycosyl transferases group 1

guanylate cyclases

Hexokinase

Hydroxymethylglutaryl-coenzyme A synthase

Lyase

vitamin-B 12 dependent methionine synthase

mRNA capping enzyme

arylamine N-acetyltransferase

nucleoside diphosphate kinase

glucosaminyl N-deacetylase/N-sulphotransferase

Myristoyl-CoA:protein N-myristoyltransferase

NNMT/PNMT/TEMT methyltransferase family

Nucleotidyl transferase

6-O-methylguanine DNA methyltransferase

Orotidine phosphate decarboxylases

O-methyltransferase

OTCase/ATCase

phenylalanine and histidine ammonia-lyases

poly(ADP-ribose) polymerase

Phosphatidate cytidylyltransferase

phosphoenolpyruvate carboxykinase

pfkB family carbohydrate kinase

Phosphofructokinase

Phosphoglycerate kinases

phosphoinositol-3-kinases

phosphatidylinositol-4-phosphate 5-kinase

eukaryotic protein kinases

polyprenyl synthetases

protein prenyltransferases

Purine/pyrimidine phosphoribosyl transferases

Phosphoribosyl pyrophosphate synthetase

6-pyruvoyl tetrahydropterin synthase

Pyridoxal-dependent decarboxylase

Pyridoxal-dependent decarboxylase conserved domain

pyridoxine kinases

pyruvate-kinase

Rhodanese

Ribosomal RNA adenine dimethylases

S-adenosylmethionine synthetase

SAICAR synthetase

Serine hydroxymethyltransferase

sialyltransferases

sterol O-acyltransferases

SpoU rRNA Methylase family

Squalene and phytoene synthases

serine/threonine dehydratases

sulfotransferases

Transaldolase

Trehalose-6-phosphate synthase domain

Tetrapyrrole (Corrin/Porphyrin) Methylases.

thymidine kinase

thiopurine methyltransferase

Thiamine Pyrophosphate requiring enzymes

Transglutaminase family

Transketolase

thymidylate synthase

ubiE/COQ5 methyltransferase family

UDP-glycosyltransferase

vitamin-K dependent gamma carboxylase

Oxidoreductases:

D-isomer specific 2-hydroxyacid dehydrogenase

3-beta hydroxysteroid dehydrogenase/isomerase

3-hydroxyacyl-CoA dehydrogenase

Acyl-CoA dehydrogenases

Zinc-containing alcohol dehydrogenases

adrenodoxin oxidoreductase

AhpC/TSA antioxidant enzyme family

aldehyde dehydrogenases

aldo/keto reductases

billiverdin reductase family

C-4 methyl sterol oxidase

C-5 cytosine-specific DNA methylase

cyclooxygenases

copper amine oxidases

FAD/NAD-binding Cytochrome reductase

D-amino acid oxidases

Molybdopterin binding domain in dehydrogenase

fatty acid desaturases

Dihydrofolate reductase

E1 dehydrogenases

Glutamate/Leucine/Phenylalanine/Valine dehydrogena

FAD-dependent glycerol-3-phosphate dehydrogenase

FMN-dependent dehydrogenase

Flavin-binding monooxygenase-like

Glucose-6-phosphate dehydrogenase

glutathione peroxidases

GMC oxidoreductases

IMP dehydrogenase/GMP reductase

Isocitrate and isopropylmalate dehydrogenases

lactate/malate dehydrogenase

lipoxygenase

NAD dependent epimerase/dehydratase family

NAD-dependent glycerol-3-phosphate dehydrogenase

NADH dehydrogenases

NADH-ubiquinone/plastoquinone oxidoreductase chain

Nitroreductase family

NO Synthase

Oxidoreductase FAD/NAD-binding domain

Delta 1-pyrroline-5-carboxylate reductase

6-phosphogluconate dehydrogenases

Alanine dehydrogenase/pyridine nucleotide transhyd

Oxidoreductase molybdopterin binding domain

ribonuclease reductases

steroid 5-alpha reductases

short-chain dehydrogenase/reductases

Succinate dehydrogenase cytochrome b subunit

Tetrahydrofolate dehydrogenase/cyclohydrolase

UDP-glucose/GDP-mannose dehydrogenases

Hydrolases:

alpha/beta hydrolases

acid ceramidase

acylphosphatase

acyl-transferase

adenosine deaminase

S-adenosyl-L-homocysteine hydrolase

AdoMet decarboxylase

amidases

arginases

Asparaginase

aspartyl proteases

astacin/m12a metalloproteases

Prenyl protease 2

Eukaryotic carbonic anhydrases

carboxylesterase

Clp family of ATP-dependent proteases

2′, 3′ cyclic nucleotide 3′ phosphodiesterase

cytidine deaminases

disintegrin

dUTPase

esterases

Fructose-1-6-bisphosphatase

Alpha-L-fucosidase

metalloprotease family

Glycosyl hydrolase family 1

hyaluronidases

GTP cyclohydrolase I

haloacid dehalogenase-like hydrolases

hemoglobinase

heparanase

histone deacetylases

insulinase

lipoprotein lipase et al

lysophospholipases

peptidase family m17

metalloprotease family M41

leishmanolysin family of metalloproteases

M24 proteases

matrix metalloproteases

mutT/8-OXO-dGTPase

neprilysin family of proteases

nucleotide pyrophosphatase (alkaline phosphodieste

procollagen N-proteinase

3′5′-cyclic nucleotide phosphodiesterase

ArgE/DapE/Acyl/Cpg2 family

Phosphorylase family

phospholipase A2

phospholipase C

phospholipase D

Porphobilinogen deaminase

pyrophosphatases

prolyl oligopeptidases

pyrimidine-nucleoside phosphorylases

GTPase-activators for Ras-like GTPases

renaldipeptidase

ADAM family of metalloprotease

serine carboxypeptidases

subtilase family of proteases

Sulfatase

Thioesterase domain

Thiolase

trehalase

trypsin-like serine proteases

Uracil-DNA glycosylase

Zinc carboxypeptidases

Zinc proteases

Isomerases:

enoyl-CoA hydratase/isomerase

sterol isomerase

Glucosamine-6-phosphate isomerase

Glyoxalase

Mannose-6-phosphate isomerase (faml)

methylacyl-CoA racemase

Macrophage migration inhibitory factor (MIF)

Phosphoglucose isomerase

phosphoglucomutase/phosphomannomutase

Phosphoglycerate mutase family

Triosephosphate isomerase

tRNA pseudouridine synthase

Other Enzymes and Receptors:

phorbol ester/DAG binding domain

phospholipid scramblase

Nuclear hormone receptors

G-protein coupled receptors

Serine/threonine kinases

Tyrosine kinases

Dual specificity kinases

Gene ID 15986

The human 15986 sequence (SEQ ID NO: 1) known also as Calcium/Calmodulin (CaM) CaMKI-like protein kinase (gi|9837341), is approximately 1579 nucleotides long including untranslated regions. The coding sequence, located at about nucleic acids 88 to 1161 in SEQ ID NO:1, encodes a 357 amino acid protein (SEQ ID NO:2).

As assessed by TaqMan analysis, 15986 mRNA was expressed at increased levels in a breast tumor pool (single sample pool of 3 tumors each) when compared to a normal breast tissue pool. Further TaqMan analysis on oncology tissue panels indicated that 15986 mRNA was expressed in 4/6 breast, in 3/6 ovary, in 3/5 lung and in 1/5 colon tumor samples when compared to three samples each of their respective normal tissue samples.

TaqMan analysis on an angiogenesis panel (fetal vs. adult tissues) indicated that 3/12 fetal tissues expressed higher levels of 15986 mRNA than 32/34 adult tissues. In particular, 15986 mRNA levels were higher in 1/1 fetal heart samples vs. 7/7 adult heart samples. TaqMan analysis on another angiogenesis panel (hemangiomas and necrotic and angiogenic tumors) indicated that 15986 mRNA was elevated in 4/8 hemangiomas vs. 1/1 normal skin tissue samples.

15986 mRNA expression levels were also elevated in 1/4 breast, in 1/3 colon, and in 3/3 lung necrotic tumors vs.1/1 sample of their respective normal tissues and in 1/1 Wilm's tumor vs. 1/1 normal kidney tissue sample.

On a TaqMan panel evaluating in vitro endothelial cell proliferation, arrest and tube formation (EC/PAT), 15986 mRNA was elevated in 2/3 human umbilical vein endothelial cell (HUVEC) tube formation samples on matrigel vs. 1/1 arrested HUVEC sample.

TaqMan analysis of an expanded breast cancer panel indicated that 15986 mRNA expression was increased in 8/11 breast invasive ductal carcinoma tumors vs. 4/4 normal breast tissue samples. In addition, 15986 mRNA expression was increased in 2/5 breast metastases vs. 5/5 normal tissue samples from the site of metastasis and 4/4 normal breast tissue samples.

On an expanded lung panel, TaqMan analysis indicated that 15986 mRNA expression was increased in 13/33 primary lung tumors vs. 6/6 normal lung tissue samples.

As assessed by in situ hybridization (ISH), 15986 was expressed in primary ovary and breast cancers. Normal breast and ovarian tissues are negative for expression. Some angiogenic tissues such as fetal kidney also showed expression of 15986.

15986, a CamKI-like protein kinase, is 82% homologous to human (sp|Q14012), rat (sp|Q63450) and mouse (gi|15928726) Ca2+/Calmodulin-dependent PKI. 15986 was shown to be active in an in vitro kinase assay, and it was demonstrated that the kinase activity is dependent on Ca2+/Calmodulin (Blood 2000 96(9): 3215-3223).

15986 is involved in cytokine signal transduction pathways resulting in the activation of CREB and CREM. IL-8, one cytokine for which 15986 has been shown to be involved, is implicated in angiogenesis, tumor growth, and metastasis in a number of cancers, including melanoma and prostate cancer. CREB activation has been shown to be critical to tumor proliferation in a number of different settings (Blood 2000, 96, 3215-3223; Endocrinology 2002, 143, 3651-7; J Biol Chem 1996, 271, 20930-4; Biometals 1998, 11, 331-43; J Biol Chem 1999, 274, 10086-93; Crit Rev Immunol 2001, 21, 275-86 and Mol Cell Biochem 2000, 212, 29-34).

Due to 15986 function and expression, inhibitors of 15986 activity would inhibit tumor proliferation, angiogenesis and metastasis. Modulators of 15986 activity would be useful in treating human cancers, including but not limited to breast cancer, lung cancer and tumor angiogenesis. 15986 polypeptides of the present invention are useful in screening for modulators of 15986 activity.

Gene ID 2188

The human 2188 sequence (SEQ ID NO:3) known also as serum/glucocorticoid regulated kinase-like (SGKL), is approximately 2391 nucleotides long including untranslated regions. The coding sequence, located at about nucleic acids 22 to 1512 in SEQ ID NO:3, encodes a 496 amino acid protein (SEQ ID NO:4).

As assessed by TaqMan analysis, 2188 mRNA was expressed at increased levels in a colon tumor pool (single sample pool of 3 tumors each) when compared to a normal colon tissue pool. Further TaqMan analysis on an oncology panel indicated that 2188 mRNA was expressed in 3/6 breast, in 6/6 lung and in 2/5 colon tumors when compared to three samples each of their respective normal tissue, although expression was generally high in colon.

TaqMan analysis on an angiogenesis panel (fetal vs. adult tissues) indicated that 2188 mRNA expression was higher in 4/12 fetal tissues vs. 34/34 adult tissues. In particular, 2188 mRNA expression levels were high in 2/2 fetal liver and in 2/2 fetal kidney samples. TaqMan analysis on another angiogenesis panel (hemangiomas and necrotic and angiogenic tumors) indicated that 2188 mRNA expression was increased in 2/8 hemangiomas when compared to normal skin tissue and increased in necrotic tumors (1/3 ovary and 3/3 lung) relative to single samples of their respective normal tissues.

On a TaqMan panel evaluating in vitro endothelial cell proliferation, arrest and tube formation (EC/PAT), 2188 mRNA expression was elevated in 1/3 human umbilical vein endothelial cell (HUVEC) tube formation samples on matrigel vs. 2/2 arrested HUVEC cell samples.

TaqMan analysis of an expanded lung cancer panel indicated that 2188 mRNA expression was increased in 3/20 lung tumors with little or no expression in 4/4 normal lung tissue samples.

In situ hybridization experiments were designed to examine primary lung tumors, normal lung, and fetal tissues representative of angiogenic tissues. The results from the ISH experiments are consistent with the data observed by Taqman analysis.

SGK1 transcription is induced by the following agonists/conditions: Serum; Glucocorticoid; Mineralocorticoid; FSH; LH; Vasoactive intestinal polypeptide; Carbachol; TGF-β; GMCSF; FMLP; TNF-α; Thrombin; Lipopolysaccharides; Hypertonicity; High glucose; and Neuronal injury or excitotoxicity. However, SGKL transcription is not induced by serum or glucocorticoids (Lang & Cohen, Science STKE 2001, 108, re17, 1-11). SGK1, SGK2, and SGKL are activated by stimulation of cells by IGF-1 and oxidative stress. SGK1 activation by hydrogen peroxide is fully inhibited by wortmannin or LY294002, whereas SGK2 and SGKL activation is only partially inhibited. All three SGKs are activated by PDK1.

Known substrates for SGK1 include GSK3β, B-Raf, FKHRL1 (cell survival) and Nedd4 (ub ligase for ENaC, controls cell volume). Known substrates for SGKL include FKHRL1 and GSK-3β (BBRC 2002, 293, 1191-6). SGK1, SGK2, and SGKL upregulate Kv1 voltage-gated K (+) channels, although the direct substrate is not known. This effect may regulate epithelial transport, cell proliferation, and neuromuscular excitability (Pflugers Arch 2002, 445, 60-6). IGF-1 induced proliferation appears to require this regulation (Pflugers Arch 2002, 445, 625-34).

2188 is an SGK protein kinase downstream of phosphatidyl-inositol-3-kinase (PI3K), regulating cell proliferation, apoptosis protection, and possibly cell volume. It acts independently of the AKTs due to its distinct localization following activation. 2188 is activated following stimulation by IL-3, IGF-1, or EGF. Inhibition of 2188 would prevent tumor cell proliferation and promote apoptosis at least in part by activation of FKHR-mediated pathways.

Due to 2188 function and expression, modulators of 2188 activity would be useful in treating human cancers, including but not limited to colon cancer, lung cancer and tumor angiogenesis. 2188 polypeptides of the present invention are useful in screening for modulators of 2188 activity.

Gene ID 20743

The human 20743 sequence (SEQ ID NO:5), also known as ribosomal protein kinase B (RSK-B), is approximately 3131 nucleotides long including untranslated regions. The coding sequence, located at about nucleic acids 66 to 2384 of SEQ ID NO:5, encodes a 772 amino acid protein (SEQ ID NO:6).

As assessed by TaqMan analysis, 20743 mRNA was expressed at low levels in most tissues. 20743 mRNA was highly expressed in human umbilical vein endothelial cells (HUVEC), coronary smooth muscle cells (SMC), brain and erythroid cells. 20743 mRNA was expressed at a 36-fold increased level in a primary colon tumor pool (single sample pool of 3 tumors each) when compared to a normal colon tissue pool.

Further TaqMan analysis on an oncology panel indicated that 20743 mRNA was expressed at 5 to 24-fold increased levels in 4/5 primary breast tumor samples vs. 2/3 normal breast samples, at 2 to 12-fold increased levels in 3/6 primary ovary tumor samples vs. 2/3 normal ovary samples, and at 150 to 210-fold increased levels in 2/4 primary colon tumors vs. 2/3 normal colon samples.

20743 mRNA also showed expression in 5/5 lung tumor samples with no expression in 2/2 normal lung samples.

20743 mRNA was expressed at 7-fold increased levels in a pooled colon liver metastases sample when compared to a normal liver sample and at 15-fold increased levels in proliferating human microvascular endothelial cells (HMVECs) when compared to arrested HMVECs.

TaqMan analysis on an expanded colon cancer tissue panel indicated that 20743 mRNA was expressed at 2 to 6-fold increased levels in 5/13 primary colon tumor samples vs. 4/6 normal colon samples. 20743 mRNA had very low expression in 6/6 liver samples, with higher expression in 3/3 colon metastases to liver.

In a TaqMan analysis on an ovarian cancer panel where cancer metastases are compared to their primary tumors, 20743 mRNA was expressed at 3 to 17-fold increased levels in 6/7 metastases when compared to their respective primary ovarian tumors.

In a TaqMan analysis on a PI3Kinase panel, 20743 mRNA expression was reduced 2 to 30-fold after 12 hrs of treatment with ˜8× the EC50 of phosphotidylinositol 3-kinase in the LNCaP, MDA-MB-468, MCF-7 Tet-Off and NCI-H125 cancer cell lines.

On a breast cancer cell model panel, 20743 mRNA expression in MCF10A cells was increased 4-fold by 8 hrs treatment of 10 ng/ml EGF and was increased 3-fold by 3 hrs treatment with 10 nM IGF1.

Finally, on an expanded lung cancer TaqMan panel, 20743 mRNA was expressed at increased levels (2-49 fold) in 11/33 primary lung tumor samples vs. 6/6 normal lung tissue samples.

The in situ hybridization experiments for 20743 indicate that this target is expressed primarily in primary colon and ovarian tumors, and colon metastases to the liver. Primary lung tumors express this gene, but at lower levels and in only a subset of tumors.

RSK-B has been identified as a novel ribosomal S6 kinase family member that is bound to p38 mitogen-activated kinase (MAPK) alpha in a yeast two-hybrid assay (J. Biol. Chem. 273(45): 29661-29671). RSK-B was found to be activated by p38alpha, as well as by ERK1, suggesting that RSK-B could represent a convergence point between the p38alpha and ERK1 MAPK pathways. The MAPK cascades are involved in the transduction of signals from mitogens and cellular stresses into appropriate cellular responses and are required for many functions including cell proliferation, differentiation, and survival (Nature 410: 37-40). The transcription factors cyclic AMP response element-binding protein (CREB) and c-fos were shown to be RSK-B substrates and RSK-B controlled CREB and AP-1 activities. A variety of stimuli, including tumor necrosis factor alpha, epidermal growth factor, phorbol 12-myristate 13-acetate and ionomycin, have been shown to induce phosphorylation and persistent activation of RSK-B through both p38 and ERK pathways (J. Biol. Chem. 275(31): 23549-23558, 2000). Studies in primary embryonic fibroblasts from RSK-B (also known as MSK-2) knockout mice showed that RSK-B is required for the mitogenic and stress-induced phosphorylation of transcription factors CREB and ATF-1 (Mol. Cell. Biol. 22(8): 2871-2881, 2002).

20743 mRNA is expressed at increased levels in ovarian cancer metastases when compared to matched primary tumors. 20743 mRNA expression is increased in MCF10A cells by EGF and IGF1 treatment and decreased by inhibition of the PI3kinase signaling pathway, suggesting that 20743 is a downstream effector of growth/survival signals. 20743 is a known player in the mitogenic and stress-induced phosphorylation of transcription factors CREB and ATF-1. Inhibiting 20743 would block mitogen and stress-induced activating phosphorylation of CREB, thereby inhibiting tumor growth.

Due to 20743 function and expression, modulators of 20743 activity would be useful in treating human cancers, including but not limited to ovarian cancer. 20743 polypeptides of the present invention are useful in screening for modulators of 20743 activity.

Gene ID 9148

The human 9148 sequence (SEQ ID NO:7), also known as NAD(P)H dehydrogenase [quinone] (Quinone reductase 1) (QR1), DT-diaphorase (DTD) or Menadione reductase, is approximately 2447 nucleotides long including untranslated regions. The coding sequence, located at about nucleic acids 51 to 875 of SEQ ID NO:7, encodes a 274 amino acid protein (SEQ ID NO:8).

As assessed by TaqMan analysis, 9148 mRNA expression was increased in pooled colon tumors (single sample pool of 3 tissue samples), pooled lung tumors and pooled ovarian tumors relative to their respective pooled normal tissues. Other tissues showing high 9148 mRNA expression levels included coronary smooth muscle, human umbilical vein endothelial cells (HUVECs), and primary osteoblasts.

Further TaqMan analyses on oncology panels indicated that 9148 mRNA was expressed at increased levels in breast, lung, ovarian and colon tumors relative to their normal tissues. 9148 mRNA was expressed at very high levels in proliferating human microvascular endothelial cells (HMVECs) and in the human colorectal carcinoma cell line HCT116.

In an expanded colon cancer panel, 9148 mRNA expression was increased in hyperplastic colon samples, adenomatous polyps, adenomas and adenocarcinomas relative to normal colon samples. 9148 mRNA expression was also increased in colon metastases to the liver when compared to normal colon and normal liver.

9148 encodes DT-diaphorase, an oxygen-independent reductase that participates in cytotoxic detoxification (Free Radic Res 2002;36(6):695-9). Upregulation of DT-diaphorase in tumors is currently being taken advantage of to activate bioreductive anti-tumor drugs (Curr Pharm Des 2002;8(15): 1319-33). However, DT-diaphorase may also metabolize toxins increased as a result of oncogenic signaling (Carcinogenesis 2000;21(10):1813-9). Therefore, selective inhibition of 9148 may result in an increase in naturally occurring cytotoxic compounds in hypoxic tumors, resulting in inhibition and/or reduction of tumor growth.

Due to 9148 function and expression, modulators of 9148 activity would be useful in treating human cancers, including but not limited to breast, lung and ovarian cancers. 9148 polypeptides of the present invention are useful in screening for modulators of 9148 activity.

Gene ID 9151

The human 9151 sequence (SEQ ID NO:9), also known as L-iditol-2 dehydrogenase (sorbitol dehydrogenase), is approximately 1808 nucleotides long including untranslated regions. The coding sequence, located at about nucleic acids 142 to 1215 of SEQ ID NO:9, encodes a 357 amino acid protein (SEQ ID NO:10).

As assessed by TaqMan analysis, 9151 mRNA expression was increased in pooled colon, pooled lung and pooled prostate tumors relative to their respective pooled normal tissues. Other tissues expressing high levels of 9151 mRNA included normal skeletal muscle, pituitary gland, brain, HUVECs and erythroid cells.

Further TaqMan analyses on oncology panels indicated that 9151 mRNA expression was increased in breast, lung and colon tumors relative to their normal tissues. 9151 mRNA was also expressed at very high levels in normal liver and in the human colorectal carcinoma cell line HCT116. In an expanded colon cancer panel, 9151 mRNA expression was increased in hyperplastic colon samples, adenomatous polyps, adenomas and adenocarcinomas relative to normal colon samples. 9151 mRNA expression was also increased in colon metastases to the liver when compared to normal colon.

9151 encodes L-iditol-2 dehydrogenase (sorbitol dehydrogenase). Sorbitol dehydrogenase catalyzes the second of the 2-step polyol pathway, converting sorbitol (derived from dietary sources or glucose metabolism) to fructose (J Med Chem 2002;45:511-28). The function of sorbitol dehydrogenase in most tissues is unknown, although the polyol pathway likely plays an important role in the etiology of diabetic complications, and therefore it is important in glucose metabolism (Genomics 1997;46:86-92). Inhibition of 9151 may adversely affect glucose metabolism in cancer cells, resulting in an inhibition and/or reduction in tumor growth.

Due to 9151 function and expression, modulators of 9151 activity would be useful in treating human cancers, including but not limited to breast, colon and lung cancers. 9151 polypeptides of the present invention are useful in screening for modulators of 9151 activity.

Gene ID 9791

The human 9791 sequence (SEQ ID NO:11), also known as cholinephosphate cytidylyltransferase A, is approximately 1286 nucleotides long including untranslated regions. The coding sequence, located at about nucleic acids 46 to 1149 of SEQ ID NO: 11, encodes a 367 amino acid protein (SEQ ID NO:12).

As assessed by TaqMan analysis, 9791 mRNA was expressed at higher levels in colon tumor and lung tumor pools with respect to their normal tissue pools. 9791 mRNA was also highly expressed in HUVECs and skeletal muscle.

Further TaqMan analyses on oncology panels indicated that 9791 mRNA was expressed at increased levels in breast, ovary and lung tumors when compared to their respective normal tissue samples. TaqMan analysis on an angiogenesis panel (fetal vs. adult tissues) indicated that 9791 mRNA was highly expressed in fetal tissues including liver, adrenal, kidney, heart and umbilical cord relative to most adult angiogenic tissue samples. TaqMan analysis on an additional angiogenesis panel (hemangiomas, necrotic and angiogenic tumors) indicated that 9791 mRNA was expressed at higher levels in hemangiomas and necrotic tumors (breast, colon and lung tumors) relative to their normal controls.

In an expanded lung tumor panel, TaqMan analysis indicated that 9791 mRNA was increased in squamous cell carcinoma and small cell lung cancer tumors when compared to normal lung tissue samples. TaqMan analysis on xenograft-friendly cancer cell lines indicated that 9791 mRNA is expressed in all cancer cell lines evaluated, with particularly high levels of expression in MCF7, ZR75, DLD1 and SW620.

In situ hybridization (ISH) analysis of 9791 indicated increased expression of 9791 mRNA in breast and lung tumors relative to normal controls. In addition, hemangiomas and angiogenic fetal tissues expressed 9791 mRNA.

The CDP-choline pathway represents an important mechanism in inducing and amplifying phosphatidate (PA) and diacylglycerol (DAG) production from phosphatidylcholine by phospholipase D (PLD). The CDP-choline pathway is upregulated in response to Ras transformation. 9791 represents the rate-limiting step of this pathway. Downregulation of this pathway results in growth inhibition and apoptosis. Therefore, inhibition of 9791 would also result in growth inhibition and apoptosis.

Due to 9791 function and expression, modulators of 9791 activity would be useful in treating human cancers, including but not limited to tumor angiogenesis, breast and lung cancers. 9791 polypeptides of the present invention are useful in screening for modulators of 9791 activity.

Gene ID 44252

The human 44252 sequence (SEQ ID NO:13), also known as rabkinesin-6 (RAB6-interacting kinesin-like protein), is approximately 2972 nucleotides long including untranslated regions. The coding sequence, located at about nucleic acids 28 to 2700 of SEQ ID NO:13, encodes a 890 amino acid protein (SEQ ID NO:14).

As assessed by TaqMan analysis, 44252 mRNA expression was restricted to HUVECs, hemangioma, osteoblasts, neutrophils, erythroid cells and ovarian, colon, and lung tumor pools (single sample pool of 3 tissue isolates) with undetectable expression in normal colon and lung pools (single sample pool of 3 tissue samples).

Further TaqMan analyses on oncology panels indicated that 44252 mRNA was expressed at increased levels in 3/5 breast, in 4/5 ovary, in 4/6 lung and in 3/5 colon tumors when compared to their respective normal tissue samples (3/3 breast, 3/3 ovary, 2/2 lung, 3/3 colon). In addition, 44252 mRNA was expressed at higher levels in proliferating HMVECs when compared to arrested HMVEC.

TaqMan analysis on an angiogenesis panel (fetal vs. adult tissues) indicated that 44252 mRNA was highly expressed in fetal tissues including liver (2/2), kidney (2/2), and heart (1/1)) relative to adult angiogenic tissue samples (32/34). 44252 mRNA was only expressed in an endometrium tumor and a uterine tumor from the adult panel. TaqMan analysis on an additional angiogenesis panel (hemangiomas, necrotic and angiogenic tumors) indicated that 44252 mRNA was expressed at higher levels in 2/8 hemangiomas relative to 1/1 normal skin tissue samples. 44252 was also expressed at higher levels in necrotic tumors (2/3 colon and 3/4 ovarian tumors) relative to their normal controls (1/1 colon and 1/1 ovary). 44252 mRNA was also expressed at higher levels in Wilm's tumor (1/1) relative to the normal control (kidney (1/1)).

In an expanded ovary tumor panel, TaqMan analysis indicated that 44252 mRNA expression was increased in 5/11 serous ovarian tumors when compared to 5/5 normal ovary tissue samples. In addition, 44252 mRNA expression was increased in 1/3 clear cell ovarian cancer tumor sample when compared to 5/5 normal lung tissue samples. 44252 mRNA was also expressed at higher levels in fallopian tube primary tumor samples (2/2) when compared to normal fallopian tube tissue samples (2/2).

TaqMan analysis of the application of the PI3K inhibitor LY294002 demonstrated that 44252 was downregulated by PI3K inhibition after 12 h in 3/4 breast cancer cell lines evaluated. Taqman analysis also demonstrated that 44252 was upregulated in proliferating lung HMVEC (3/3) or HUVEC (1/1) relative to their normal controls (lung HMVEC (3/3) or HUVEC (1/1).

The ISH analysis for 44252 indicated very restricted expression. There was modest to high expression in breast and ovarian tumors and fetal liver. No expression was seen in normal ovary or breast tissue. This ISH data confirms the clinical TaqMan data.

In later stages of mitosis Rabkinesin-6 localized to the spindle midzone and appeared on the midbodies during cytokinesis. The functional significance of this localization during M phase was revealed by antibody microinjection studies which resulted exclusively in binucleate cells, showing a complete failure of cytokinesis. These results substantiate a crucial role for Rabkinesin-6 in late anaphase B and/or cytokinesis, clearly distinct from the role of MKLP-1 (Mol Cell Biol 2001, 21, 2944-55). Rabkinesin-6 accumulates in mitotic cells where it localizes to the midzone of the spindle during anaphase and to the cleavage furrow and midbody during telophase. Overexpression of Rabkinesin-6 causes a cell division defect resulting in cell death. Microinjection of anti-rabkinesin-6 antibodies results in binucleate cells with a defect in cleavage furrow formation and thus cytokinesis (EMBO J, 2000, 19, 5711-9). Rabkinesin-6 was shown to be one of 5 novel cytokine-responsive genes identified from endothelial cells activated by monocyte-conditioned medium or tumor necrosis factor alpha (Blood 1999, 93, 3418-31).

44252 is a critical kinesin involved in cleavage furrow formation in telophase. Upregulation of 44252 in tumors would be indicative of a greater mitotic index and would be expected to promote progression through cytokinesis. Much of the literature shows expression of 44252 in endothelial cells, indicating a key role in angiogenesis. Inhibition of 44252 would be expected to prevent endothelial cell proliferation in support of tumor vascularization. In addition, tumors that express 44252 would have their cell cycle progression significantly slowed.

Due to 44252 function and expression, modulators of 44252 activity would be useful in treating human cancers, including but not limited to tumor angiogenesis, breast and ovarian cancers. 44252 polypeptides of the present invention are useful in screening for modulators of 44252 activity.

Gene ID 14184

The human 14184 sequence (SEQ ID NO:15), also known as serine/threonine protein kinase EMK, is approximately 2946 nucleotides long including untranslated regions. The coding sequence, located at about nucleic acids 408 to 2645 of SEQ ID NO:15, encodes a 745 amino acid protein (SEQ ID NO:16).

Transcriptional profiling identified increased 14184 mRNA expression in a panel of lung tumors versus normal lung samples. As assessed by TaqMan analysis, 14184 mRNA expression was broad in normal tissues, with the highest expression in pancreas and central nervous system tissues. TaqMan analysis indicated that 14184 mRNA was upregulated in lung, breast and ovarian tumors. Further TaqMan analyses indicated a decrease in 14184 mRNA expression in NCI-H125 cells (p53 mutant) in which wild-type p53 has been reintroduced.

The drosophila homologue (Par-1) of the protein encoded by 14184 was recently shown to be a signaling component of the wnt pathway (Nature Cell Bio 2001;3:628-636), which is thought to play a role in solid tumor development and progression. Specifically, Par-1 potentiates B-catenin signaling while at the same time inhibiting the JNK pathway. Perturbation of this pathway by overexpression of 14184 would be expected to contribute to tumorigenesis by driving abnormal wnt signaling.

Due to 14184 function and expression, modulators of 14184 activity would be useful in treating human cancers, including but not limited to breast, ovarian and lung cancers. 14184 polypeptides of the present invention are useful in screening for modulators of 14184 activity.

Gene ID 42461

The human 42461 sequence (SEQ ID NO:17), also known as helicase-like transcription factor (HLTF); SWI/SNF related, matrix associated, actin dependent regulator of chromatin, subfamily a, member 3; or ATPase, DNA-binding protein (HIP116), is approximately 5457 nucleotides long including untranslated regions. The coding sequence, located at about nucleic acids 313 to 3342 of SEQ ID NO:17, encodes a 1009 amino acid protein (SEQ ID NO:18).

Transcriptional profiling identified increased 42461 mRNA expression in a subset of lung tumors (>2× in 10/21 tumors) versus normal lung samples. As assessed by TaqMan analysis, 42461 mRNA was upregulated in 6/6 lung tumors and in 2/6 breast tumors as compared to their respective normal tissue samples. Further TaqMan analysis on an expanded panel indicated 42461 mRNA was upregulated in 18/35 lung tumor samples as compared to normal lung tissue samples. Additional analysis showed decreased 42461 mRNA expression in NCI-H125 cells (p53 mutant) in which wild-type p53 has been reintroduced.

In situ hybridization experiments conducted on 42461 showed that primary lung tumors are positive for 42461 (6/7 tumor samples vs. 0/2 normal lung tissue samples). No expression was seen in normal lung epithelial cells.

42461 (HLTF) is a member of the SWI/SNF family of chromatin remodeling proteins that play a role in transcriptional activation and repression. While a number of family members, including HLTF, have been implicated as tumor suppressors, the emerging picture appears to be much more complex. Brg1, which appears to function with the Rb protein in tumor suppression, has been shown to have increased levels of expression in advanced gastric carcinomas. It is also critical for the survival and proliferation of F9 carcinoma cells suggesting that, like many chromatin regulators, its role in the cell extends beyond a tumor suppressor function. Overexpression of 42461 in tumor cells may disrupt a critical balance required for controlled chromosome maintenance and predispose cells towards a tumorigenic phenotype.

Due to 42461 function and expression, modulators of 42461 activity would be useful in treating human cancers, including but not limited to breast and lung cancers. 42461 polypeptides of the present invention are useful in screening for modulators of 42461 activity.

Gene ID 8204

The human 8204 sequence (SEQ ID NO:19), known also as adenine phosphoribosyltransferase (APRT), is approximately 841 nucleotides long including untranslated regions. The coding sequence, located at about nucleic acids 72 to 614 of SEQ ID NO:19, encodes a 180 amino acid protein (SEQ ID NO:20).

As assessed by TaqMan analysis, 8204 mRNA was expressed at higher levels in colon and lung tumors (single sample pool of 3 tumors) with respect to normal colon and lung pools, respectively. 8204 mRNA was also highly expressed in coronary smooth muscle cells (SMC), human umbilical vein endothelial cells (HUVECs) and erythroid cells.

Further TaqMan analysis on oncology tissue panels indicated that 8204 mRNA was expressed at increased levels in 5/6 breast, in 3/6 lung, and in 4/5 colon tumors when compared to their respective normal tissue samples (3/3 breast, 3/3 lung, 3/3 colon). 8204 mRNA was also expressed at increased levels in proliferating human microvascular endothelial cells (HMVECs) relative to arrested HMVECs.

TaqMan analyses performed using an angiogenesis panel (fetal vs. adult tissues) indicated that 8204 mRNA was expressed at increased levels in fetal liver (2/2), in adrenal (2/2), in kidney (2/2) and in heart (1/1) relative to adult angiogenic tissue samples (31/34), including adult heart (7/7). TaqMan analysis using an additional angiogenesis panel (hemangiomas and necrotic and angiogenic tumors) indicated that 8204 mRNA was expressed at higher levels in 2/8 hemangiomas relative to 1/1 normal skin tissue samples. 8204 mRNA was also expressed at higher levels in necrotic tumors (1/4 breast, 3/3 colon, and 3/3 lung tumors).

In an expanded lung tumor panel, TaqMan analyses indicated that 8204 mRNA was increased in 15/33 lung tumors when compared to 6/6 normal lung tissue samples. Increased expression of 8204 mRNA was particularly noted in lung SCC (7/10).

In a colon ACA tumor panel, TaqMan analyses indicated that 8204 mRNA was increased in 2/15 colon primary tumors when compared to 6/6 normal colon tissue samples and 3/5 colon tumor metastasis samples when compared to either 6/6 normal colon tissue samples or 6/6 normal liver tissue samples. In a colon tumor metastasis panel, TaqMan analysis indicated that 8204 mRNA was increased in 4/4 colon primary tumors when compared to 3/3 normal colon tissue samples and in 14/16 samples of colon tumor metastasis to the liver when compared to either 3/3 normal colon tissue samples or 3/3 normal liver tissue samples. 8204 mRNA expression was also increased in 7/14 samples of colon tumor metastasis to other sites when compared to 3/3 normal colon tissue samples.

In an expanded breast tumor panel, TaqMan analysis indicated that 8204 mRNA was increased in 1/15 breast primary tumors when compared to 4/4 normal breast tissue samples or in 11/15 breast primary tumors when compared to 2/4 normal breast tissue samples. Breast metastases (4/5) had increased mRNA levels of 8204 when compared to their normal tissue sample controls (5/5).

TaqMan analysis of 8204 mRNA expression in an “endothelial cell in vitro proliferation, arrest and tube formation” panel indicated increased expression in proliferating HUVEC (1/1) and lung HMVEC (3/3) when compared to arrested HUVEC (1/1) and lung HMVEC (3/3).

Based on the TaqMan data, tissues from primary lung, colon, and colon metastases to the liver as well as several angiogenic tissues were used in in situ hybridization experiments. Expression of 8204 was confirmed in each of these tumors types by ISH. Normal lung and normal colon were modestly positive by ISH, confirming the TaqMan data.

Deficiency of the enzyme adenine phosphoribosyltransferase (APRT) has been associated with hypersensitivity to the mutagenic effects of ethyl methanesulfonate and 254 nm ultraviolet radiation in clone 707 of Friend mouse erythroleukemia (FEL) cells. The mutagen hypersensitivity of APRT-deficient cells may be the result of decreased efficiency in the excision repair processes due to reduced levels of ATP (Br J Biomed Sci 1997, 54, 174-80).

Cancer cells upregulate DNA repair pathways to compensate for a lack of checkpoint controls within the cell cycle. Maintenance of ATP levels within the cells is important to maintain efficiency of excision repair processes. Inhibition of 8204 would target cancer cells by preventing this compensation mechanism.

Due to 8204 function and expression, modulators of 8204 activity would be useful in treating human cancers, including but not limited to breast, colon and lung cancers, and tumor angiogenesis. 8204 polypeptides of the present invention are useful in screening for modulators of 8204 activity.

Gene ID 7970

The human 7970 sequence (SEQ ID NO:21), known also as AAA ATPase, is approximately 1748 nucleotides long including untranslated regions. The coding sequence, located at about nucleic acid 79 to 1164 of SEQ ID NO:21, encodes a 361 amino acid protein (SEQ ID NO:22).

As assessed by TaqMan analysis, 7970 mRNA showed low to moderate expression in a broad range of tissues. 7970 mRNA showed highest expression in tissues of the central nervous system and in the heart. Clinical samples showed an increased expression of 7970 mRNA in 6/7 lung tumors, 2/6 breast tumors, 2/6 primary colon tumors, and 2/4 colon metastases as compared to the normal controls.

In situ hybridization experiments indicated that 7970 mRNA expression was increased in the epithelium of lung tumor samples (4/5) versus normal controls.

AAA Atpases constitute a large family of proteins with a wide array of functions, including membrane trafficking, transcriptional regulation, proteolysis, cytoskeletal regulation, and cell cycle control. Overexpression of 7970 in tumor epithelium confers a selective growth advantage on these cells. Therefore, inhibition of 7970 in tumor cells would lead to growth arrest and an eventual reduction in tumor size. Due to 7970 mRNA expression in lung, breast and primary colon tumors, along with its functional role, modulators of 7970 activity would be useful in treating human cancers. 7970 polypeptides of the present invention are useful in screening for modulators of 7970 activity.

Gene ID 25552

The human 25552 sequence (SEQ ID NO:23), known also as a methyltransferase, is approximately 1529 nucleotides long including untranslated regions. The coding sequence, located at about nucleic acid 57 to 980 of SEQ ID NO:23, encodes a 307 amino acid protein (SEQ ID NO:24).

As assessed by TaqMan analysis, 25552 mRNA showed ubiquitous expression in colon tumor when compared to normal colon tissues. Further TaqMan experiments indicated that 25552 mRNA showed elevated expression in 2/8 Breast, in 3/9 Ovary, in 8/8 Lung, 5/8 Colon, and in 3/4 metastases tissues.

In situ hybridization experiments indicated that 25552 mRNA was expressed in 4/8 primary colon tumors and in 3/3 metastases tissues.

Dysregulated expression of 25552 is potentially protecting cancer cells from undergoing cell death. Due to 25552 mRNA expression in breast, lung and colon tumors, along with its functional role, modulators of 25552 activity would be useful in treating human cancers. 25552 polypeptides of the present invention are useful in screening for modulators of 25552 activity.

Gene ID 21657

The human 21657 sequence (SEQ ID NO:25), known also as short-chain dehydrogenase/reductase, is approximately 1249 nucleotides long including untranslated regions. The coding sequence, located at about nucleic acid 53 to 1006 of SEQ ID NO:25, encodes a 317 amino acid protein (SEQ ID NO:26).

As assessed by TaqMan analysis, 21657 mRNA showed broad expression in normal tissues, with highest expression in the pancreas and ovary. Further TaqMan experiments indicated that 21657 mRNA expression was increased in 2/6 breast tumors and in 2/7 lung tumors relative to their normal controls.

In situ hybridization experiments indicated that 21657 mRNA was expressed in the tumor epithelium of 4/5 primary colon tumors and of 4/4 colon metastases tissues. Lung and breast tissues showed modest expression of 21657 mRNA that was equivalent to the normal controls.

Members of the short-chain dehydrogenase/reductase (SDR) family function in the metabolism of retinoid and steroid hormones, both of which have been demonstrated to have dramatic effects on tumor cell growth. Therefore, overexpression of 21657 affects hormone homeostasis in tumor cells, conferring a selective growth advantage on those cells. Due to 21657 mRNA expression in breast and lung tumors, along with its functional role, modulators of 21657 activity would be useful in treating human cancers. 21657 polypeptides of the present invention are useful in screening for modulators of 21657 activity.

Gene ID 26492

The human 26492 sequence (SEQ ID NO:27), known also as Diphosphoinositol polyphosphate phosphohydrolase (DIPP), is approximately 4396 nucleotides long including untranslated regions. The coding sequence, located at about nucleic acid 148 to 534 of SEQ ID NO:27, encodes a 128 amino acid protein (SEQ ID NO:28).

As assessed by TaqMan analysis, 26492 mRNA was expressed in the heart, skeletal muscle, and kidney. 26492 mRNA also showed increased expression in 4/8 lung tumor samples, in 3/8 breast tumor samples, in 3/8 primary colon tumors, and in 2/4 liver metastases when compared with normal control tissues.

In situ hybridization experiments indicated specific expression of 26492 mRNA in the tumor epithelium of 5/5 lung tumors, of 3/3 colon tumors, and of 1/1 breast tumors.

DIPP family members dephosphorylate diphosphoinositol polyphosphates and act as molecular switches in the cell. Among the processes controlled by these molecules are intracellular trafficking, DNA repair, and apoptosis. It was recently demonstrated in an ovarian tumor cell lines that PP-InsP5 is involved in induction of apoptosis. Therefore, overexpression of 26492 would expect to reduce the levels of PP-InsP5 in the cell.

Reduced PP-InsP5 in the cell results in resistance to apoptosis, which is a hallmark phenotype of tumor cells. Due to 26492 mRNA expression in lung, breast and primary colon tumors, along with its functional role, modulators of 26492 activity would be useful in treating human cancers. 26492 polypeptides of the present invention are useful in screening for modulators of 26492 activity.

Gene ID 2411

The human 2411 sequence (SEQ ID NO:29), known also as G-protein coupled receptor, is approximately 1382 nucleotides long including untranslated regions. The coding sequence, located at about nucleic acid 61 to 1284 of SEQ ID NO:29, encodes a 407 amino acid protein (SEQ ID NO:30).

As assessed by TaqMan analysis, 2411 mRNA expression was increased 46 fold in pooled lung tumors relative to the pooled normal lung and increased 2 fold in pooled breast tumors relative to normal breast. 2411 mRNA was expressed at high levels in coronary smooth muscle cells, HUVEC, brain cortex, dorsal root ganglia, ovary, prostate and pancreas.

In oncology TaqMan panels, 2411 mRNA expression was increased in 4/6 lung tumors (5 to 25 fold) relative to 3 normal lung samples, and approximately 15 fold in 2 colon liver metastases relative to normal liver.

In expanded colon tumor panels, 2411 mRNA was expressed at increased levels in 6/6 colon liver metastases (5 to 18 fold) relative to 5 normal liver samples. In expanded colon liver metastases panels, 2411 mRNA was expressed at increased levels in 10/17 colon liver metastases (3 to 20 fold) relative to 3 normal liver samples.

2411 mRNA was also detected in 2/3 colon tumors, in 1/1 colon polyps and in 3/3 colon liver metastases by in situ hybridiztion. 2411 mRNA was detected in 2/3 lung tumors, but not in 1/1 normal lung tissues. 2411 mRNA expression was increased in some colon liver metastases and lung tumors relative to normal liver and lung, respectively.

2411 encodes an unnamed G-protein coupled receptor, a family of receptors involved in the regulation of diverse cellular functions, including growth, differentiation and apoptosis (Oncogene 2001; 20:1530-31). Many activated forms of G-protein coupled receptors are known to transform cells by initiating constitutive oncogenic signaling. Therefore, selective inhibition of 2411 in cancers expressing high levels of this gene may disrupt these oncogenic signaling networks, resulting in an inhibition and/or reduction in tumor growth. Due to 2411 mRNA expression in lung, breast, colon and liver tumors, along with its functional role, modulators of 2411 activity would be useful in treating human cancers. 2411 polypeptides of the present invention are useful in screening for modulators of 2411 activity.

Gene ID 15088

The human 15088 sequence (SEQ ID NO:31), known also as a unnamed protein product, is approximately 3492 nucleotides long including untranslated regions. The coding sequence, located at about nucleic acid 104 to 3007 of SEQ ID NO:31, encodes a 967 amino acid protein (SEQ ID NO:32).

As assessed by TaqMan analysis, 15088 mRNA was expressed at high levels in pooled colon tumor when compared to normal colon tissues. In addition, 15088 mRNA expression was increased 25 fold in the pooled ovary tumors relative to the pooled normal ovaries. 15088 mRNA was expressed at moderately high levels in artery, diseased aorta, heart, pituitary gland, spinal cord and hypothalamus.

In oncology TaqMan panels, 15088 mRNA expression was increased in 3/4 colon tumors (3 to 9 fold) relative to 3 normal colons, in 5/6 ovary tumors (4 to 27 fold) relative to 3 normal ovaries, and in 4/6 lung tumors (3 to 6 fold) relative to 3 normal lungs.

In expanded colon tumor panels, 15088 mRNA was expressed at increased levels in 3/15 colon tumors (2 to 6 fold) and in 2/7 colon metastases (2.5 and 4 fold) relative to 6 normal colons. 15088 mRNA was also expressed at low levels in 6 normal livers. In expanded colon metastases panels, 15088 mRNA was expressed at increased levels in 4/4 colon tumors (2 to 23 fold) and in 13/17 colon to liver metastases (3 to 50 fold) relative to 2 normal colons. 15088 mRNA was also expressed at low levels in 3 normal livers.

In expanded ovary tumor panels, 15088 mRNA was expressed at increased levels in 10/12 ovary tumors and in 1/1 ovary to lung metastases (4 to 65 fold) relative to 4 normal ovaries.

In situ hybridization experiments detected 15088 mRNA in 3/5 colon tumors, in 4/5 colon metastases, and in 3/6 ovary tumors.

15088 encodes an unnamed G-protein coupled receptor, a family of receptors involved in the regulation of diverse cellular functions, including growth, differentiation and apoptosis (Oncogene 2001; 20:1530-31). Expression of some activated forms of G-protein coupled receptors are known to transform cells by initiating constitutive oncogenic signaling. Therefore, selective inhibition of 15088 in cancers expressing high levels of this gene may disrupt these oncogenic signaling networks, resulting in an inhibition and/or reduction in tumor growth. Due to 15088 mRNA expression in ovary, colon, lung and liver tumors, along with its functional role, modulators of 15088 activity would be useful in treating human cancers. 15088 polypeptides of the present invention are useful in screening for modulators of 15088 activity.

Gene ID 1905

The human 1905 sequence (SEQ ID NO:33), known also as GPCR-rhodopsin, is approximately 1342 nucleotides long including untranslated regions. The coding sequence, located at about nucleic acid 179 to 1180 of SEQ ID NO:33, encodes a 333 amino acid protein (SEQ ID NO:34).

As assessed by TaqMan analysis, 1905 mRNA expression was highest in brain cortex, lymph node, tonsil, liver fibrosis, spleen, lung cancer and skeletal muscle. 1905 mRNA was higher in colon and lung tumors (single sample pool of 3 tissue isolates) when compared to expression in normal colon and lung (single sample pool of 3 tissue samples).

Further TaqMan analyses on oncology tissue panels indicated that 1905 mRNA was expressed at increased levels in 6/8 breast, in 4/5 ovary, in 6/6 lung and in 6/6 colon tumors when compared to their respective normal tissue samples (3/3 breast, 2/2 ovary, 3/3 lung, 3/3 colon).

On a TaqMan colon panel, 1905 mRNA was expressed at increased levels in 6/15 colon tumors when compared to normal colon (5/6). 1905 mRNA was also increased in 5/6 colon metastases to the liver relative to normal liver (6/6) or normal colon (5/6). TaqMan analysis on a colon metastasis panel demonstrated that 1905 mRNA was increased in colon tumors (3/4) relative to normal colon (3/3). 1905 mRNA was also increased in colon metastasis to the liver (14/17) when compared to either normal colon (3/3) or normal liver (3/3).

In situ hybridization experiments confirmed that 1905 mRNA was elevated in colon tumors, including 2/2 adenomas, 1/1 dysplastic tissue, 1/3 tumors, and 5/6 colon metastases relative to 3/3 normal colon and 3/3 normal liver.

Chemokines represent a large family of polypeptide signaling molecules that are notable for their role in chemotaxis, leukocyte homing, directional migration, and GPCR activation. Chemokines have recently been implicated in tumor progression and metastasis (J Invest Dermatol 2002, 118, 915-22). GPCR stimulation induces proliferation and prevents apoptosis of hormone independent prostate cancer cells, indicating their role in the progression of prostate cancer (J Urol 2002, 167, 1458-63).

1905 is involved in tumor progression and metastasis by promoting proliferation, cell survival, and cell migration. Due to 1905 mRNA expression in lung, breast and colon tumors, along with its functional role, modulators of 1905 activity would be useful in treating human cancers. 1905 polypeptides of the present invention are useful in screening for modulators of 1905 activity.

Gene ID 28899

The human 28899 sequence (SEQ ID NO:35), known also as 1-acyl-sn-glycerol-3-phosphate acyltransferase, is approximately 1832 nucleotides long including untranslated regions. The coding sequence, located at about nucleic acid 192 to 1322 of SEQ ID NO:35, encodes a 376 amino acid protein (SEQ ID NO:36).

As assessed by TaqMan analysis, 28899 mRNA was expressed at 4-68 fold increased levels in 6/6 primary breast tumor samples when compared to 2/3 normal breast samples; at 3-83 fold increased levels in 5/7 primary lung tumor samples when compared to 3/3 normal lung samples; and at 2-23 fold increased levels in 4/4 primary colon tumor samples when compared to 2/3 normal colon samples. 28899 mRNA was expressed at a 22-fold increased level in the primary breast tumor pool when compared to the normal breast tissue pool, at a 15-fold increased level in the primary colon tumor pool when compared to the normal colon tissue pool, and at a 466-fold increased level in the primary lung tumor pool when compared to the normal lung tissue pool. Other tissue samples which express 28899 include tissues from the central nervous system, kidney and heart. 28899 mRNA was also expressed in many ovarian cancer cell lines, including SKOV3, A2780, OVCAR3, MDA2774 and ES-2.

In situ hybridization experiments indicated that 28899 mRNA was expressed in ovary, colon and breast tumors.

LPA acyltransferase, is the enzyme in lipid metabolism which converts lysophosphatidic acid (LPA) to phosphatidic acid (PA). LPA and PA are potent mitogens for various cell types which function through EDG family and ras pathway, respectively. In addition, LPA and PA can induce the chemotactic migration in endothelial cells. It has been shown that overexpression of LPA acyltransferase in A549 lung epithelial carcinoma and ECV304 endothelial cells results in a significant increase of cytokine expression. High levels of 28899 expression in tumors results in increased intracellular levels of phosphatidic acid, which in turn drives tumor cell proliferation, and possibly invasion. Due to 28899 mRNA expression in breast, lung and colon tumors, along with its functional role, modulators of 28889 activity would be useful in treating human cancers. 28899 polypeptides of the present invention are useful in screening for modulators of 28899 activity.

Gene ID 63380

The human 63380 sequence (SEQ ID NO:37), known also as delta7-delta8 sterol isomerase, is approximately 931 nucleotides long including untranslated regions. The coding sequence, located at about nucleic acid 53 to 673 of SEQ ID NO:37, encodes a 206 amino acid protein (SEQ ID NO:38).

As assessed by TaqMan analysis, 63380 mRNA was expressed at 4->100 fold increased levels in 4/4 primary colon tumor samples when compared to 3/3 normal colon samples; at 5-14 fold increased levels in 2/2 colon liver metastases samples when compared to 1/1 normal liver sample; and at 2-31 fold increased levels in 3/6 primary lung tumor samples when compared to 3/3 normal lung samples. 63380 mRNA was expressed at a 6-fold increased level in the primary colon tumor pool when compared to the normal colon tissue pool, and at a 4-fold increased level in the primary lung tumor pool when compared to the normal lung tissue pool. Other tissues which express 63380 at high levels include erythroid cells, pancreas and brain. 63380 mRNA was also expressed in many ovarian cancer cell lines, including A2780, OVCAR3, HEY, MDA2774 and ES-2.

Emopamil-binding protein (EBP), also known as sterol isomerase, is an integral membrane protein of the endoplasmic reticulum. It is a high affinity binding protein for the antiischemic phenylalkylamine Ca2+ antagonist [3H]emopamil and the photoaffinity label [3H]azidopamil. EBP is similar to sigma receptors and may be a member of a superfamily of high affinity drug-binding proteins in the endoplasmic reticulum of different tissues. EBP shares structural features with bacterial and eukaryontic drug transporting proteins. It has four putative transmembrane segments and contains two conserved glutamate residues which may be involved in the transport of cationic amphiphilics. Another prominent feature of EBP is its high content of aromatic amino acid residues (>23%) in its transmembrane segments. These aromatic amino acid residues are involved in the drug transport by the P-glycoprotein. Mutations in EBP cause Chondrodysplasia punctata 2 (CDPX2; also known as Conradi-Hunermann syndrome).

63380 is potentially an endoplasmic reticulum membrane enzyme involved in sterol biosynthesis. Inhibition of 63380 is predicted to inhibit tumor growth. Due to 63380 mRNA expression in lung and colon tumors, along with its functional role, modulators of 63380 activity would be useful in treating human cancers. 63380 polypeptides of the present invention are useful in screening for modulators of 63380 activity.

Gene ID 33935

The human 33935 sequence (SEQ ID NO:39), known also as Glycosyl Transferase I, is approximately 2590 nucleotides long including untranslated regions. The coding sequence, located at about nucleic acid 11 to 1489 of SEQ ID NO:39, encodes a 492 amino acid protein (SEQ ID NO:40).

As assessed by TaqMan analysis, 33935 mRNA expression was at very low to moderate levels in a broad range of tissues. Highest expression of 33935 mRNA was seen in the brain cortex. Increased 33935 mRNA expression levels was observed in 6/7 lung tumors, in 2/6 breast tumors, in 2/6 primary colon tumors, and in 2/4 colon metastases as compared to the normal controls.

In situ hybridization experiments indicated that 33935 mRNA expression levels were increased in the epithelium of lung tumor samples (4/5) versus normal controls.

33935 is a member of the glycosyltransferase I family. Members of this family transfer UDP, ADP, GDP or CMP linked sugars to a variety of substrates, including glycogen, fructose-6-phosphate and lipopolysaccharides. A change in the structure of glycans added to glycoproteins and glycolipids is a common feature of the change to malignancy. Increased expression of 33935 in tumor cells results in the increased glycosylation of key substrates that control cell growth, survival, and invasive capacity. This in turn contributes to the malignant cellular phenotype observed in tumors. Due to 33935 mRNA expression in lung, breast and colon tumors, along with its functional role, modulators of 33935 activity would be useful in treating human cancers. 33935 polypeptides of the present invention are useful in screening for modulators of 33935 activity.

Gene ID 10480

The human 10480 sequence (SEQ ID NO:41), known also as CoA transferase, is approximately 3337 nucleotides long including untranslated regions. The coding sequence, located at about nucleic acid 99 to 1661 of SEQ ID NO:41, encodes a 520 amino acid protein (SEQ ID NO:42).

As assessed by TaqMan analysis, 10480 mRNA showed broad, variable expression, with highest normal expression in brain cortex and erythroid cells. 10480 mRNA showed increased expression in 6/6 lung tumors when compared to normal control tissues.

In situ hybridization experiments showed low to high expression of 10480 mRNA in the tumor epithelium of 4/5 lung samples and of 4/6 breast samples.

Reduction of 10480 expression in tumor cells by transfection with siRNA oligos specific for 10480 resulted in cell growth inhibition. RNAi studies in 3 different cell lines (A549, HT29, and HCT116) showed levels of growth inhibition upon 10480 expression reduction comparable to those seen in a PLK1 positive control.

Succinyl-CoA:3-ketoacid-CoA transferase (SCOT) functions as the first step in catabolism of ketone bodies that are formed by β-oxidation of fatty acids. A number of lines of evidence from profiling experiments point to increased use of fatty acids as an energy source by tumor cells, which results in higher levels of ketone bodies within these cells. SCOT is potentially increased in tumor cells as a mechanism for metabolizing ketone bodies, which can be detrimental to cells at high concentrations. Due to 10480 mRNA expression in lung tumors, along with its functional role, modulators of 10480 activity would be useful in treating human cancers. Small molecule inhibitors of 10480 would be expected to have an effect similar to that seen with RNAi on tumor cell growth, and are therefore attractive cancer therapeutic candidates. 10480 polypeptides of the present invention are useful in screening for modulators of 10480 activity.

Gene ID 12686

The human 12686 sequence (SEQ ID NO:43), known also as DEAD-type helicases, is approximately 1864 nucleotides long including untranslated regions. The coding sequence, located at about nucleic acid 67 to 1518 of SEQ ID NO:43, encodes a 483 amino acid protein (SEQ ID NO:44).

As assessed by TaqMan analysis, 12686 mRNA expression was highest in HUVEC, skeletal muscle, pancreas, brain cortex, and erythroid cell samples. 12686 mRNA expression was highest in colon and lung tumors (single sample pool of 3 tissue isolates) when compared to expression in normal colon and lung samples (single sample pool of 3 tissue samples).

Further TaqMan analyses on oncology tissue panels indicated that 12686 mRNA was expressed at increased levels in 3/6 breast, in 3/6 ovary, in 6/6 lung and in 4/5 colon tumors when compared to their respective normal tissue samples (3/3 breast, 3/3 ovary, 3/3 lung, 3/3 colon). In addition, 12686 mRNA was expressed at higher levels in proliferating HMVEC when compared to arrested HMVEC.

TaqMan analysis using an angiogenesis panel indicated that 12686 mRNA was highly expressed in fetal tissues including liver (1/2), adrenal (2/2), kidney (2/2), and heart (1/1) samples relative to adult angiogenic tissue samples (4/4). Also, TaqMan analysis using another angiogenesis panel also indicated that 12686 mRNA was expressed at higher levels in 1/6 hemangiomas relative to 2/2 normal skin tissue samples. 12686 mRNA was also expressed at higher levels in necrotic tumors (2/2 lung and 1/1 breast tumors) relative to their normal controls (1/1 lung and 1/1 breast). 12686 mRNA was expressed at higher levels in Wilm's tumor (3/3) relative to the normal control (kidney (1/1) and other kidney tumors (3/3)). TaqMan analysis on the EC/PAT panel also demonstrated that 12686 mRNA was upregulated in proliferating lung HMVEC (1/1) or HUVEC (2/2) relative to their arrested controls (lung HMVEC (1/1) or HUVEC (2/2)).

12686 is a nucleolar helicase involved in the biogenesis of the 40S ribosomal subunits. Inhibition of 12686 would lead to a depletion of active 80S ribosome, resulting in a reduction of protein synthesis within the cell. Due to 12686 mRNA expression in lung, breast, ovary and colon tumors, along with its functional role, modulators of 12686 activity would be useful in treating human cancers. 12686 polypeptides of the present invention are useful in screening for modulators of 12686 activity.

Gene ID 25501

The human 25501 sequence (SEQ ID NO:45), known also as SAM-dependent methyltransferase, is approximately 1971 nucleotides long including untranslated regions. The coding sequence, located at about nucleic acid 16 to 1527 of SEQ ID NO:45, encodes a 503 amino acid protein (SEQ ID NO:46).

As assessed by TaqMan analysis, 25501 mRNA expression was highest in brain cortex, glial cells, and ovarian, prostate, and colon tumors. 25501 mRNA expression was higher in ovarian and colon tumors (single sample pool of 3 tissue isolates) when compared to expression in normal ovarian and colon tissue samples (single sample pool of 3 tissue samples).

Further TaqMan analysis on oncology tissue panels indicated that 25501 mRNA was expressed at increased levels in 2/6 breast, in 2/5 ovary, in 8/8 lung and in 4/6 colon tumors when compared to their respective normal tissue samples (3/3 breast, 2/2 ovary, 3/3 lung, 3/3 colon). In addition, 25501 mRNA was expressed at higher levels in proliferating HMVEC when compared to arrested HMVEC.

TaqMan analysis using an angiogenesis panel indicated that 25501 mRNA was highly expressed in fetal tissues (3/3) relative to adult tissue samples (2/3), especially fetal heart (1/1) relative to adult heart (1/1). Further TaqMan analysis using another angiogenesis panel indicated that 25501 mRNA was expressed at higher levels in 2/3 hemangiomas relative to 1/1 normal skin tissue samples. 25501 mRNA was also expressed at higher levels in Wilm's tumor (2/2) relative to the normal control (kidney (1/1)) and other kidney tumors (1/1).

On a colon TaqMan panel, 25501 mRNA was expressed at increased levels in 14/15 colon tumors when compared to normal colon (4/6). 25501 mRNA expression was also increased in 6/6 colon metastases to the liver relative to normal liver (6/6) or normal colon (4/6).

TaqMan analysis on the EC panels also demonstrated that 25501 mRNA was upregulated in proliferating lung HMVEC (2/2), cardiac HMVEC (2/2) or HUVEC (2/2) relative to their arrested controls (lung HMVEC (2/2), cardiac HMVEC (2/2) or HUVEC (2/2)).

In situ hybridization experiments confirmed the TaqMan results by demonstrating that 25501 mRNA upregulation was tumor specific in lung, colon, and in the angiogenic Wilm's tumor and fetal adrenal, but no change was found in breast tumors from the already high expression in normal epithelium.

25501 is a member of the UPF0020 family, indicative of a putative RNA methylase activity. Inhibition of 25501 may have an impact on protein translation. Due to 25501 mRNA expression in lung, colon, breast, and ovary tumors, along with its functional role, modulators of 25501 activity would be useful in treating human cancers. 25501 polypeptides of the present invention are useful in screening for modulators of 25501 activity.

Gene ID 17694

The human 17694 sequence (SEQ ID NO:47), known also as a methionyl-tRNA synthetase, is approximately 2800 nucleotides long including untranslated regions. The coding sequence, located at about nucleic acid 24 to 2726 of SEQ ID NO:47, encodes a 900 amino acid protein (SEQ ID NO:48).

As assessed by TaqMan analysis, 17694 mRNA expression was increased 10.5 fold in a pooled colon tumor sample relative to a pooled normal colon sample. 17694 mRNA expression was also elevated 7.5 fold in a pooled lung tumor sampl relative to a pooled normal lung sample. Other tissues expressing high 17694 mRNA expression levels included HUVEC cells, heart, primary osteoblasts, pituitary gland, brain cortex and erythroid cells.

In oncology TaqMan panels, 17694 mRNA expression was increased in 3/5 breast tumors (2.5 to 7 fold) relative to 3 normal breast samples, in 4/6 lung tumors (3 to 7 fold) relative to 3 normal lung samples, and in 2/4 colon tumors (2 and 4 fold) relative to 3 normal colon samples. 17694 mRNA was also expressed at high levels in the human colorectal carcinoma cell line HCT116.

In colon metastasis TaqMan panels, 17694 mRNA expression was increased in 2/4 colon tumors (2.5 to 3.5 fold) and in 8/16 colon to liver metastases (2 to 12 fold) relative to 3 normal colon samples. 17694 mRNA was expressed at very high levels in normal liver.

In expanded lung tumor TaqMan panels, 17694 mRNA expression was increased in 6/33 lung tumors (2 to 5 fold) relative to 5 normal lungs and 1 normal lung trachea.

17694 expression was detected at low levels in 3/3 normal lungs by in situ hybridization. However, 17694 was detected at significantly higher levels in 2/3 lung adenocarcinomas, in 2/2 squamous cell carcinomas and in 1/2 poorly differentiated non-small cell carcinomas.

17694 encodes a methionyl-tRNA synthetase, a member of the aminoacyl-tRNA synthetases. This family of enzymes ligates amino acids to their cognate tRNAs which are subsequently used as substrates in peptide elongation during protein translation (Acta Biochimica Polonica (2001); 48(2):337-50). Methionyl-tRNA synthetase ligates the amino acid methionine to tRNA (Gene (1996); 178:187-9). In addition to their role in peptide elongation, aminoacyl-tRNA synthetases participate in translational fidelity, RNA processing and transcriptional/translational regulation. In addition, methionyl-tRNA synthetase is thought to participate in formation of rRNA in nucleoli (The Journal of Cell Biology (2000); 149(3):567-74).

Because 17694 expression is increased in some cancers, and because it plays a role in several essential processes associated with cell growth regulation, 17694 mRNA is an ideal target for anti-cancer drug design. Selective inhibition of 17694 results in inhibition and/or reduction of tumor growth. Due to 17694 mRNA expression in lung, colon and liver tumors, along with its functional role, modulators of 17694 activity would be useful in treating human cancers. 17694 polypeptides of the present invention are useful in screening for modulators of 17694 activity.

Gene ID 15701

The human 15701 sequence (SEQ ID NO:49), known also as Kinesin-lilke protein KIF14, is approximately 6586 nucleotides long including untranslated regions. The coding sequence, located at about nucleic acid 440 to 5386 of SEQ ID NO:49, encodes a 1648 amino acid protein (SEQ ID NO:50).

As assessed by TaqMan analysis, expression of 15701 mRNA expression was fairly restricted to epithelial tumors. 15701 mRNA was expressed at a 5-fold increased level in the breast tumor pool when compared to normal breast tissue pool, at a 10-fold increased level in the ovary tumor pool when compared to the normal ovary tissue pool, and at a 5-fold increased level in the primary colon tumor pool when compared to the normal colon tissue pool 15701 mRNA was also expressed in the lung tumor pool, while it is not expressed in the normal lung tissue pool. Other samples expressing 15701 mRNA include erythroid cells, HUVEC cells and primary osteoblasts.

In an oncology TaqMan tissue panel, 15701 mRNA was expressed at increased levels (10-154 fold) in 3/5 primary breast tumors when compared to 3/3 normal breast tissue samples. 15701 mRNA was expressed at increased levels (3-20 fold) in 5/6 primary ovary tumor samples when compared to 2/3 normal ovary samples. 15701 mRNA was expressed at detectable levels in 5/6 primary lung tumors, however 15701 expression is not detected in 3/3 normal lung tissue samples. 15701 mRNA was expressed at 4-fold increased levels in proliferating HMVEC cells when compared to arrested HMVEC cells.

In an expanded breast cancer tissue panel, 15701 mRNA was expressed at increased levels (2-131 fold) in 14/15 primary breast tumor samples when compared to 4/4 normal breast tissue samples. In a breast cancer cell model panel, 15701 mRNA was expressed at a 4-fold increased level in transformed, activated H-ras-expressing MCF10AT01 cells vs. normal MCF10A cells, and at a 5-fold increased level in transformed, activated H-ras-expressing MCF10AT3B cells when compared to normal MCF10A cells. 15701 mRNA was expressed in many breast tumor cell lines, including MCF-7, T47D, ZR-75 and MDA-MB-468. In the PI3-kinase panel, 15701 mRNA expression was decreased in MDA-MB-468, MCF-7 and LNCaP cells after 48 hours treatment with the PI3-kinase inhibitor LY294002. 15701 mRNA expression in MDA-MB-468 cells decreased 7-fold after 48 hours with LY294002 (115 uM). 15701 mRNA expression in MCF-7 cells decreased 16-fold after 48 hours with LY294002 (30 uM). 15701 mRNA expression in LNCaP cells was completely undetectable after 48 hours with LY294002 (30 uM).

15701 is the kinesin-like protein KIF14. Kinesin superfamily proteins (KIFs) have been shown to transport organelles, protein complexes and mRNAs to specific destinations in a microtubule and ATP-dependent manner. Additionally, some kinesins participate in chromosomal and spindle movements during mitosis and meiosis. 45 KIFs have been identified in humans, with many conserved in other species. 15701 is a kinesin protein that is expressed at increased levels in many epithelial tumors and may be involved in chromosome segregation during mitosis. Inhibiting 15701 is predicted to lead to a variety of mitotic defects, ultimately leading to cell death. Due to 15701 mRNA expression in lung, colon and liver tumors, along with its functional role, modulators of 15701 activity would be useful in treating human cancers. 15701 polypeptides of the present invention are useful in screening for modulators of 15701 activity.

Gene ID 53062

The human 53062 sequence (SEQ ID NO:51), known also as ser-thr protein kinase related to the myotonic dystrophy protein kinase, is approximately 5373 nucleotides long including untranslated regions. The coding sequence, located at about nucleic acid 1 to 4719 of SEQ ID NO:51, encodes a 1572 amino acid protein (SEQ ID NO:52).

As assessed by TaqMan analysis, 53062 mRNA was expressed at a 20-fold increased level in the lung tumor pool when compared to the normal lung tissue pool; at a 9-fold increased level in the primary colon tumor pool when compared to the normal colon tissue pool; and at a 2-fold increased level in the ovary tumor pool when compared to the normal ovary tissue pool. 53062 mRNA was expressed at a 2-fold increased level in the lung tumor pool when compared to the normal lung tissue pool; at a 3-fold increased level in the primary colon tumor pool when compared to the normal colon tissue pool; and at a 3-fold increased level in the ovary tumor pool when compared to the normal ovary tissue pool.

In oncology tissue TaqMan panels, 53062 mRNA was expressed at increased levels (3-17 fold) in 5/6 primary ovary tumor samples when compared to 3/3 normal ovary samples. 53062 mRNA was expressed at increased levels (3-37 fold) in 6/6 primary lung tumor samples when compared to 2/2 normal lung tissue samples. 53062 mRNA expression was increased 12-fold in a pooled colon liver metastases sample when compared to a normal liver samples.

In an expanded lung cancer tissue panel, 53062 mRNA was expressed at increased levels (2-27 fold) in 15/33 primary lung tumor samples when compared to 6/6 normal lung tissue samples.

In an expanded colon cancer tissue panel, 53062 mRNA was expressed at increased levels (7-72 fold) in 4/4 colon liver metastases samples when compared to 4/4 normal liver tissue samples.

In expanded breast cancer tissue panels, 53062 mRNA was expressed at increased levels (2-54 fold) in 11/15 primary breast tumor samples when compared to 2/4 normal breast tissue samples, however the remaining 2/4 normal breast tissue samples are the highest expressing samples on this panel.

In an oncology xenograft cell line panel, 53062 mRNA was expressed in several tumor cell lines, including DLD-1, NCI-H69, NCI-H322 and MCF-7.

53062 is the human kinase polypeptide (PKIN-20) cDNA. This kinase has mouse and rat putative orthologs. 53062 is 42% identical to human myotonic dystrophy protein kinase (DMPK, Q09013). Decreased expression of DMPK leads to the disease myotonic dystrophy, an inherited disorder in which the muscles contract but have decreasing power to relax. With this condition, the muscles also become weak and waste away. Myotonic dystrophy can cause mental deficiency, hair loss and cataracts. 53062 is not expressed in skeletal muscle and is therefore likely to have a divergent role from DMPK. 53062 is a protein kinase of unknown function with increased expression levels in 50% of primary lung cancers. Inhibition of 53062 may inhibit tumor progression. Due to 53062 mRNA expression in lung, colon, breast and ovary tumors, along with its functional role, modulators of 53062 activity would be useful in treating human cancers. 53062 polypeptides of the present invention are useful in screening for modulators of 53062 activity.

Gene ID 49908

The human 49908 sequence (SEQ ID NO:53), known also as DEAD/DEXH helicase DDX31, is approximately 3286 nucleotides long including untranslated regions. The coding sequence, located at about nucleic acid 154 to 2709 of SEQ ID NO:53, encodes a 851 amino acid protein (SEQ ID NO:54).

As assessed by TaqMan analysis, 49908 mRNA expression was increased 14 fold in pooled colon tumors relative to pooled normal colons. 49908 mRNA expression was also elevated 3 fold in pooled lung tumors relative to pooled normal lung. Other tissues expressing high 49908 mRNA levels included brain cortex and HUVEC.

In oncology TaqMan panels, 49908 mRNA expression was increased in 2/4 colon tumors (5 and 5.5 fold) relative to 3 normal colon samples and in 4/6 lung tumors (5 to 9 fold) relative to 3 normal lung samples. 49908 mRNA was also expressed at high levels in the human colorectal carcinoma cell line HCT116. In SVA colon polyp panels, 49908 mRNA expression was increased in 3/4 adenomas (2 to 3.7 fold), and in 1/5 adenocarcinomas (4.8 fold) relative to 5 normal colon samples.

In expanded colon tumor panels, there was a single outlying normal colon sample expressing moderately high 49908 levels. Omitting that sample 49908 mRNA was expressed at increased levels in 7/15 colon tumors (2 to 3.5 fold) and in 3/4 colon to liver metastases (2 to 4 fold) relative to 6 normal colon samples. 49908 mRNA was expressed at moderate levels in normal liver.

In expanded lung tumor panels, 49908 mRNA expression was increased in 10/33 lung tumors (2 to 14 fold) relative to 4 normal lung and in 1 normal lung trachea. The tumors expressing increased 49908 mRNA comprised mainly squamous cell carcinomas (8/10). 49908 mRNA was expressed at moderate levels in normal skin.

49908 encodes the DEAD/DEXH helicase DDX31, a member of the RNA helicase family. RNA helicases are characterized by 8 conserved motifs. Variations in motif II allow the RNA helicases to be classified into groups, such as DEAD, DEAH or DExH (Nature Structural Biology; (2000) 7(2):97-99). 49908 contains the conserved amino acid sequence Asp-Glu-Ala-Asp at motif II, and is therefore considered a DEAD-box RNA helicase. RNA helicases are implicated in several cellular processes involving RNA secondary structure; including transcription, pre-mRNA processing, ribosome biogenesis, RNA export, translational initiation, mitochondrial gene expression and RNA degradation. Other DEAD-box family members have been implicated in growth regulation and tumor initiation/progression as they have been found to be overexpressed in various cancers (Oncogene (2001) 20:7734-43; and The Journal of Biological Chemistry (1998) 273(33):21161-68). For example, the DEAD-box protein rck/p54 was found to be overexpressed in the majority of colorectal tumors examined. rck/p54 overexpression was implicated in increasing levels of the oncogenic protein c-Myc by enhancement of its translation efficiency and/or stabilization of its mRNA (Carcinogenesis (2001) 22(12):1965-70).

49908 expression is increased in colon and lung cancers, and because 49908 plays a role in several essential processes associated with cell growth regulation, it is an ideal target for anti-cancer drug design. Selective inhibition of 49908 potentially results in inhibition and/or reduction of tumor growth. Due to 49908 mRNA expression in lung and colon tumors, along with its functional role, modulators of 49908 activity would be useful in treating human cancers. 49908 polypeptides of the present invention are useful in screening for modulators of 49908 activity.

Gene ID 21612

The human 21612 sequence (SEQ ID NO:55), known also as SDR (short-chain dehydrogenase/reductases), is approximately 2535 nucleotides long including untranslated regions. The coding sequence, located at about nucleic acid 762 to 2018 of SEQ ID NO:55, encodes a 418 amino acid protein (SEQ ID NO:56).

As assessed by TaqMan analysis, 21612 mRNA expression was increased 4 fold in the pooled colon tumors relative to the pooled normal colons. 21612 mRNA expression was also elevated 5 fold in pooled lung tumors relative to pooled normal lungs. 21612 mRNA was expressed at high levels in diseased heart, skeletal muscle, brain cortex, ovary and prostate epithelial cells.

In oncology TaqMan panels, 21612 mRNA expression was increased in 6/7 lung tumors (2 to 5 fold) relative to 3 normal lung samples, and in 3/4 colon tumors (2 to 3 fold) relative to 3 normal colon samples.

In situ hybrization experiments indicated that 21612 mRNA was detected in 0/3 normal colons, in 1/1 hyperplastic/dysplastic colons, in 2/5 colon adenocarcinomas, in 0/1 colon metastases to the liver and in 0/3 normal livers. 21612 mRNA was detected in 1/3 lung tumors, but not in 1/1 normal lung tissues.

21612 encodes an unnamed short-chain dehydrogenase/reductase, a heterogenous superfamily of enzymes. In humans, 37 members were identified that fall into three functional classes; enzymes involved in intermediary metabolism, enzymes participating in lipid hormone metabolism, and enzymes with unknown functions (Chemico-Biological Interactions (2001) 130-2: 699-705). 21612 expression is increased in some colon and lung tumors relative to the respective normal tissue. Some of these family members have been associated with increased risk of ovarian and breast cancer. Selective inhibition of 21617 results in an inhibition and/or reduction in tumor growth. Due to 21612 mRNA expression in lung and colon tumors, along with its functional role, modulators of 21612 activity would be useful in treating human cancers. 21617 polypeptides of the present invention are useful in screening for modulators of 21617 activity.

Gene ID 38949

The human 38949 sequence (SEQ ID NO:57), known also as abhydrolase (alpha/beta hydrolases), is approximately 1298 nucleotides long including untranslated regions. The coding sequence, located at about nucleic acid 78 to 1166 of SEQ ID NO:57, encodes a 362 amino acid protein (SEQ ID NO:58).

As assessed by TaqMan analysis, 38949 mRNA was expressed at moderate levels in normal brain cortex, hypothalamus and to a lesser extent in normal ovary. In oncology TaqMan panels, 38949 mRNA was expressed in 1/1 colon to liver metastases samples.

In expanded colon panels, 38949 mRNA was expressed in 3/5 colon to liver metastases. In expanded colon metastases panels, 38949 mRNA was expressed in 2/4 colon tumors and in 8/16 colon to liver metastases.

In situ hybridization experiments idicated that 38949 mRNA was detected at low levels in 1/3 colon adenocarcinomas and in 2/4 colon to liver metastases, but not in 3 normal colon or 2 normal liver samples.

38949 encodes an unknown alpha/beta hydrolase of unknown function. The alpha/beta hydrolase fold is common to a number of hydrolytic enzymes of widely differing phylogenetic origin and catalytic function (Protein Eng 1992;5(3):197-211). 38949 expression is increased in some colon to liver metastases. The specificity of 38949 to colon to liver metastases may make it an ideal target for inhibiting colon metastases. Due to 38949 mRNA expression in colon tumors, along with its functional role, modulators of 38949 activity would be useful in treating human cancers. 38949 polypeptides of the present invention are useful in screening for modulators of 38949 activity.

Gene ID 6216

The human 6216 sequence (SEQ ID NO:59), known also as Propionyl-CoA carboxylase alpha subunit, is approximately 2423 nucleotides long including untranslated regions. The coding sequence, located at about nucleic acid 49 to 2160 of SEQ ID NO:59, encodes a 703 amino acid protein (SEQ ID NO:60).

As assessed by TaqMan analysis, 6216 mRNA expression was increased 100 fold in the pooled colon tumors relative to the pooled normal colons. 6216 mRNA expression was also elevated 2-3 fold in pooled ovarian, prostate and lung tumors relative to pooled normal tissue. 6216 mRNA was expressed at high levels in heart, kidney, adrenal gland and brain cortex.

In oncology TaqMan panels, 6216 mRNA expression was increased in 2/4 colon tumors (5 and 17 fold) relative to 3 normal colon samples, and in 2/6 lung tumors (3 and 42 fold) relative to 3 normal lung samples.

In expanded colon tumor panels, 6216 mRNA was expressed at increased levels in 3/12 colon tumors (2 to 3.5 fold) and in 2/5 colon metastases to the liver (5 and 6 fold) relative to 5 normal colon samples.

6216 mRNA was detected in 5/6 colon tumors, in 1/1 colon polyps and in 5/5 colon metastases by in situ hybridiztion. 6216 mRNA was not detected in 3 normal colons, but was detected in 2 normal livers. 6216 mRNA was detected in 2/5 lung tumors, but not in 2 normal lungs.

6216 encodes propionyl-CoA carboxylase alpha, one of two subunits that make up propionyl-CoA carboxylase, a biotin-dependent mitochondrial enzyme involved in the catabolism of branched chain amino acids, odd-numbered chain length fatty acids, cholesterol, and other metobolites (Human Mutation (1999) 14:275-82). The involvement of carboxylation reactions in the metabolism of short chain fatty acids is the main source of energy for human colonic cells (Int J Cancer (1997) 72:768-75). This may explain upregulation of this gene in some rapidly proliferating colon tumors. 6216 expression is increased in some colon and lung tumors relative to the respective normal tissue. Therefore, selective inhibition of 6216 may adversely affect metabolism in cancer cells, resulting in an inhibition and/or reduction in tumor growth. Due to 6216 mRNA expression in colon and lung tumors, along with its functional role, modulators of 6216 activity would be useful in treating human cancers. 6216 polypeptides of the present invention are useful in screening for modulators of 6216 activity.

Gene ID 46863

The human 46863 sequence (SEQ ID NO:61), known also as ANM (arginine N-methyltransferase), is approximately 2864 nucleotides long including untranslated regions. The coding sequence, located at about nucleic acid 141 to 2678 of SEQ ID NO:61, encodes a 845 amino acid protein (SEQ ID NO:62).

As assessed by TaqMan analysis, 46863 mRNA expression was increased 8 fold in pooled lung tumors relative to the pooled normal lung and 8 fold in pooled colon tumors relative to pooled normal colon. 46863 mRNA was expressed at moderately high levels in arteries, veins, coronary smooth muscle cells, HUVEC, heart, kidney, skeletal muscle, adrenal gland and erythroid cells.

In oncology TaqMan panels, 46863 mRNA expression was increased in 4/6 lung tumors (2 to 7 fold) relative to 3 normal lung samples, and in 4/4 colon tumors and in 1/2 colon to liver metastases (2.5 to 7 fold) relative to 3 normal colon samples and 1 normal liver sample.

46863 mRNA was detected in 1/5 colon tumors, in 1/1 hyperplastic colons and in 2/6 colon to liver metastases by in situ hybridization. 46863 mRNA was detected in 1/3 lung tumors but not in 1 normal lung sample by in situ hybridization.

46863 encodes an unnamed arginine N-methyltransferase. Arginine methylation is a common protein posttranslational modification suspected to regulate protein subcellular localization, protein-protein interactions and transcriptional activity. 46863 expression is increased in some colon and lung tumors relative to normal colon and lung, respectively. Selective inhibition of 46863 in cancers expressing high levels of 46863 disrupts the activity of proteins associated with various oncogenic signaling networks, resulting in an inhibition and/or reduction in tumor growth. Due to 46863 mRNA expression in lung and colon tumors, along with its functional role, modulators of 46863 activity would be useful in treating human cancers. 46863 polypeptides of the present invention are useful in screening for modulators of 46863 activity.

Gene ID 9235

The human 9235 sequence (SEQ ID NO:63), known also as Glutamine-fructose-6-phosphate transaminase, is approximately 3082 nucleotides long including untranslated regions. The coding sequence, located at about nucleic acid 123 to 2168 of SEQ ID NO:63, encodes a 681 amino acid protein (SEQ ID NO:64).

As assessed by TaqMan analysis, 9235 mRNA expression was increased in 3/5 breast tumors (2 to 15 fold) relative to 3 normal breast samples, and in 5/6 lung tumors (15 to 38 fold) relative to 3 normal lungs.

In expanded colon tumor panels, there was a single outlying normal colon sample expressing moderately high 9235 mRNA levels. Omitting that sample, 9235 expression was increased in 8/15 colon tumors (2 to 4 fold) and in 4/4 colon to liver metastases (2 to 4 fold) relative to 5 normal colon samples. 9235 mRNA was expressed at low levels in normal liver. In colon metastases panels, 9235 mRNA expression was increased in 2/4 colon tumors (2 to 3 fold) and in 9/16 colon metastases (2 to 13 fold) relative to 2 normal colon samples. 9235 mRNA was expressed at low levels in normal liver.

9235 mRNA was detected in 1/3 normal colons, in 0/2 normal jejunum, in 5/6 colon adenocarcinomas, and in 4/4 colon metastases to the liver by in situ hybridization. 9235 mRNA was detected in 2/3 lung tumors, but not in 2/2 normal lungs.

9235 encodes glututamine-fructose-6-phosphate transaminase. 9235 converts 2-5% of fructose-6 phosphate derived from glucose to glucosamine-6-phosphate using glutamine as an amide donor (Endocrinology (1995) 136(7):2809-16; and Biochimica et Biophysica Acta (2002) 1597:173-92). Glucosamine-6-phosphate is then transformed to uridine diphosphate-N acetylglucosamine (UDP-GlcNAc), a substrate used for N- and O-linked protein glycosylation. 9235 expression is increased in some breast, lung and colon tumors relative to the respective normal tissue. The membrane-spanning and ECM molecules expressed by tumors often differ quantitatively and qualitatively from the cell or tissue from which they are derived and these differences play a critical role in tumor initiation and progression. Therefore, changes in glutamine-fructose-6-phosphate transaminase activity in cancer may be critical in the synthesis of tumor-associated cell surface molecules. Selective inhibition of 9235 may alter the gylcocaylx of cancer cells, resulting in an inhibition and/or reduction in tumor growth. Due to 9235 mRNA expression in colon and lung tumors, along with its functional role, modulators of 9235 activity would be useful in treating human cancers. 9235 polypeptides of the present invention are useful in screening for modulators of 9235 activity.

Gene ID 2201

The human 2201 sequence (SEQ ID NO:65), known also as mixed lineage kinase (MLK), is approximately 2455 nucleotides long including untranslated regions. The coding sequence, located at about nucleic acids 1 to 2403 of SEQ ID NO:65, encodes an 800 amino acid protein (SEQ ID NO:66).

As assessed by TaqMan analysis, 2201 mRNA was expressed at increased levels (2 fold) in colon tumor samples vs. normal colon samples (15 fold) and in lung tumor samples vs. normal lung samples.

In an additional TaqMan panel, 2201 mRNA was expressed at increased levels in primary breast tumor samples (2.5-81 fold), in lung tumor samples (2-19 fold) and in primary colon cancer samples (3-49 fold) when compared to their respective control samples. 2201 is expressed at 1′-fold increased levels in proliferating human microvascular endothelial cells (HMVECs) when compared to arrested HMVECs.

In an expanded cancer tissue panel, 2201 mRNA is expressed at increased levels in primary colon tumor samples (2-148 fold), in primary breast tumor samples (3-15 fold) and in primary lung tumor samples (2-985 fold) when compared to their respective control samples.

In a PI3Kinase panel, 2201 mRNA expression is reduced 4-24 fold after 24 hours of treatment with approximately 8× the EC50 of phosphotidylinositol 3-kinase in LNCaP (30 uM), MDA-MB468 (115 uM), MCF 7 Tet-Off (30 uM) and NCI-H125 (150 uM) cancer cell lines.

In a breast cancer cell model panel, 2201 is increased 3-fold after an 8 hour treatment of MCF10A cells with 10 ng/ml EGF.

Mixed linkage kinases have been proposed to function as mitogen-activated protein kinase kinase kinases in pathways leading to MAPK cascade. MLKs may have a pivotal role in cell growth regulation. The expression of ZAK, a mixed linkage kinase, in mammalian cells specifically leads to the activation of the JNK/SAPK pathway as well as the activation of the transcription factor, NF-kappa B. Overexpression of the ZAK gene induces the apoptosis of a hepatoma cell line. (Biochem Biophys Res Commun 2000, Aug 11; 274 (3): 811-6).

Due to 2201 function and expression, modulators of 2201 activity would be useful in treating human cancers, including but not limited to colon and lung cancers, and tumor angiogenesis. 2201 polypeptides of the present invention are useful in screening for modulators of 2201 activity.

Gene ID 6985

The human 6985 sequence (SEQ ID NO:67), known also as carnitine O-palmitoyltransferase I, mitochondrial muscle isoform (CPTM) (CPTI), is approximately 2565 nucleotides long including untranslated regions. The coding sequence, located at about nucleic acids 20 to 2338 of SEQ ID NO:67, encodes a 772 amino acid protein (SEQ ID NO:68).

As assessed by TaqMan analysis on an oncology tissue panel, 6985 was expressed at 5-fold increased levels in the ovarian tumor pool vs. the normal ovary tissue pool and 3-fold increased levels in the colon tumor pool vs. the normal colon tissue pool. Other tissue samples in which 6985 mRNA expression levels were shown to be over-expressed include heart, skeletal muscle and brain.

In another oncology tissue panel, 6985 is expressed at 4-226 fold increased levels in 5/5 primary ovary tumor samples vs. 3/3 normal ovary samples, at 9-189 fold increased levels in 5/6 primary lung tumor samples vs. 3/3 normal lung samples, and at 12-138 fold increased levels in 2/4 primary colon tumor samples vs. 3/3 normal colon samples.

In an ovary cell model panel, 6985 is expressed in many ovarian cancer cell lines, including A2780 and OVCAR3.

6985 is a component of the carnitine system, responsible for facilitating entry of long-chain fatty acids into mitochondria for their utilization in energy-generating processes. Overexpression of 6985 in ovarian and lung tumors suggests a unique requirement for the activity of this enzyme in these cancers. Inhibition of 6985 is predicted to inhibit tumor progression.

Due to 6985 function and expression, modulators of 6985 activity would be useful in treating human cancers, including but not limited to colon, ovarian and lung cancers. 6985 polypeptides of the present invention are useful in screening for modulators of 6985 activity.

Gene ID 9883

The human 9883 sequence (SEQ ID NO:69), known also as iduronate-2-sulfatase precursor, is approximately 2297 nucleotides long including untranslated regions. The coding sequence, located at about nucleic acids 125 to 1777 of SEQ ID NO:69, encodes an 550 amino acid protein (SEQ ID NO:70).

As assessed by TaqMan analysis, 9883 mRNA expression was highest in artery, pituitary gland, brain cortex, brain hypothalamus, and dorsal root ganglion. 9883 mRNA expression was higher in ovarian, colon, and lung tumors (single sample pool of 3 tissue isolates) when compared to expression in normal ovarian, colon, and lung (single sample pool of 3 tissue samples).

Further TaqMan analyses on oncology tissue panels indicated that 9883 mRNA was expressed at increased levels in breast, lung and colon tumors when compared to their respective normal tissue samples. In addition, 9883 mRNA was expressed at higher levels in proliferating human microvascular endothelial cells (HMVEC) when compared to arrested HMVEC.

TaqMan analysis using an angiogenesis panel indicated that 9883 mRNA was highly expressed in fetal heart tissue relative to adult heart tissue samples. 9883 mRNA was also expressed at higher levels in Wilm's tumor and other kidney tumors relative to the normal kidney control. 9883 mRNA expression was high in angiogenic tumors and was expressed at increased levels in lung tumors and breast tumors relative to normal controls.

TaqMan analysis on the EC/PAT panel also demonstrated that 9883 mRNA was upregulated in tube formation for lung HMVEC and human umbilical vein endothelial cells (HUVEC) relative to their arrested or proliferating controls.

L-Iduronate rich glycosaminoglycans regulate the activity of growth factors and the ability of tumor cells to metastasize. The degree of sulfation is critical to at least some of the function of the L-iduronate rich glycosaminoglycans. Upregulation of 9883 is seen in tube formation TaqMan panel, consistent with a migratory/invasion role for 9883 in endothelial cells. Inhibition of 9883 would result in a decrease in metastasis in tumors reliant on these interactions.

Due to 9883 function and expression, modulators of 9883 activity would be useful in treating human cancers, including but not limited to breast, ovarian, colon and lung cancers, and tumor angiogenesis. 9883 polypeptides of the present invention are useful in screening for modulators of 9883 activity.

Gene ID 12238

The human 12238 sequence (SEQ ID NO:71), known also as activated p21cdc42Hs kinase, is approximately 4548 nucleotides long including untranslated regions. The coding sequence, located at about nucleic acids 451 to 3729 of SEQ ID NO:71, encodes an 1092 amino acid protein (SEQ ID NO:72).

As assessed by TaqMan analysis, 12238 mRNA expression levels were elevated in colon and lung tumors (single sample pool of 3 tumors) with respect to normal colon and lung (single sample pool of 3 tissue samples). 12238 mRNA was also highly expressed in brain cortex, pituitary, hypothalamus, skeletal muscle, and erythroid cells (single sample pool of 3 tissue samples).

Further TaqMan analysis on an oncology tissue panels indicated that 12238 mRNA was expressed at increased levels in 1/5 breast, in 2/6 ovarian, in 5/6 lung, and in 4/5 colon tumors when compared to their respective normal tissue samples (3/3 breast, 3/3 ovary, 3/3 lung, 3/3 colon). 12238 mRNA was expressed at increased levels in proliferating HMVEC relative to arrested HMVEC.

TaqMan analysis using an angiogenesis panel (fetal vs. adult tissues) indicated that 12238 mRNA was expressed at increased levels in fetal liver (2/2), in kidney (2/2), in heart (1/1), and in umbilical cord (1/2) relative to adult angiogenic tissue samples (29/34).

TaqMan analysis using another angiogenesis panel (hemangiomas and necrotic and angiogenic tumors) indicated that 12238 mRNA was expressed at higher levels in 3/8 hemangiomas relative to 1/1 normal skin tissue samples. 12238 was also expressed at higher levels in necrotic tumors (2/3 breast, 3/3 colon, and 3/3 lung tumors) when compared with normal controls (1/1 breast, 1/1 colon, and 1/1 lung)

In an expanded lung tumor panel, TaqMan analysis indicated that 12238 mRNA was increased in 17/33 lung tumors when compared to 6/6 normal lung tissue samples.

In a colon ACA tumor panel, TaqMan analysis indicated that 12238 mRNA was increased in 4/15 colon primary tumors when compared to 6/6 normal colon tissue samples and in 1/5 colon tumor metastasis samples when compared to either 6/6 normal colon tissue samples or 6/6 normal liver tissue samples.

In an expanded breast tumor panel, TaqMan analysis indicated that 12238 mRNA was increased in 2/15 breast primary tumors when compared to 4/4 normal breast tissue samples or in 15/15 breast primary tumors when compared to 2/4 normal breast tissue samples. Breast metastases (2/5) had increased 12238 mRNA levels when compared to their normal tissue sample controls (5/5).

TaqMan analysis of 12238 mRNA expression in the “endothelial cell in vitro proliferation, arrest and tube formation” panel indicated increased expression in lung HMVEC (2/3) when compared to lung HMVEC (3/3). High expression of 12238 mRNA was also seen late in tube formation (16 h) with HUVEC (1/1) grown on matrigel relative to early time points (2 & 6 h) and controls grown on plastic (proliferating & arrested HUVEC).

In situ experiments performed on 12238 show expression of 12238 in lung and colon primary tumors, as well as in a number of angiogenic tissues. The experiments showed some variation in the number of positive samples. However, the specimens that are positive show clearly that this gene is expressed in tumor cells.

Cdc42 regulates signaling pathways that control cell morphology, migration, endocytosis, and cell cycle progression. Overexpression of the cdc42-binding domain of ACK1 completely reversed v-Ha-Ras-induced malignant phenotypes such as focus formation and anchorage/serum-independent growth of NIH3T3 cells (Oncogene 1999, 18, 7787-93). Melanoma chondroitin sulfate proteoglycan (MCSP) is a cell-surface antigen that has been implicated in the growth and invasion of melanoma tumors. MCSP-induced spreading of melanoma cells is dependent upon active cdc42, ACK1 and tyrosine phosphorylation of p130cas (Nat Cell Biol 1999, 1, 507-13). ACK1 phosphorylates and activates the guanine nucleotide exchange factor Dbl, which in turn directs the Rho family GTP-binding proteins. ACK1 acts as a mediator of EGF signals to Rho family GTP-binding proteins through the phosphorylation and activation of GEFs such as Dbl (Biochem Biophys Res Commun 2001, 284, 470-7). Accumulation of the GTP-bound form of Rho and Rac within the cell paralleled ACK1-dependent tyrosine phosphorylation of Dbl. ACK1 may act as a regulator of Dbl, which in turn activates Rho family proteins (Biochem Biophys Res Commun 2000, 268, 141-7). GEF activity of Ras-GRF1 toward Ha-Ras was augmented after tyrosine phosphorylation by ACK1 (J Biol Chem 2000, 275, 29788-93).

12238 mediates the role of cdc42 in cell morphology, migration, endocytosis, and cell cycle progression. 12238 phosphorylates and activates Rho family GEFs, including Dbl and Ras-GRF1. 12238 activity induces increases in Rho-GTP and Rac-GTP. Inhibition of 12238 would reverse transformation of cells in which 12238 is activated, resulting in tumor regression.

Due to 12238 function and expression, modulators of 12238 activity would be useful in treating human cancers, including but not limited to colon and lung cancers, and tumor angiogenesis. 12238 polypeptides of the present invention are useful in screening for modulators of 12238 activity.

Gene ID 18057

The human 18057 sequence (SEQ ID NO:73), an uncharacterized protein, is approximately 1859 nucleotides long including untranslated regions. The coding sequence, located at about nucleic acids 218 to 1627 of SEQ ID NO:73, encodes an 469 amino acid protein (SEQ ID NO:74).

In a TaqMan panel, 18057 mRNA was expressed at increased levels in pooled colon tumors (5 fold) relative to pooled normal colon and in pooled breast tumors (3 fold) relative to pooled normal breast. 18057 mRNA was also expressed in pooled lung tumors, but not at all in pooled normal lung. 18057 mRNA was expressed at moderately high levels in kidney, brain, cortex and hypothalamus.

In an oncology TaqMan panel, 18057 mRNA expression was increased in colon to liver metastases (4.5 and 5 fold) relative to normal colons. 18057 mRNA was not detected in normal liver. 18057 mRNA expression was also increased in breast tumors (2 and 4 fold) relative to normal breasts and in ovary tumors (3.5 to 9 fold) relative normal ovaries. 18057 mRNA was expressed in lung tumors at relatively moderate to high levels, but was not expressed in normal lungs.

18057 mRNA expression is increased in some colon, breast, ovary and lung tumors. 18057 encodes an unnamed G-protein coupled receptor, a family of receptors involved in the regulation of diverse cellular functions, including growth, differentiation and apoptosis (Oncogene 2001; 20:1530-31). Expression of some activated forms of G-protein coupled receptors is known to transform cells by initiating constitutive oncogenic signaling (Oncogene 2001; 20:147-55). Therefore, selective inhibition of 18057 in cancers expressing high levels of this gene may disrupt these oncogenic signaling networks, resulting in an inhibition and/or reduction in tumor growth.

Due to 18057 function and expression, modulators of 18057 activity would be useful in treating human cancers, including but not limited to breast, ovarian, colon and lung cancers. 18057 polypeptides of the present invention are useful in screening for modulators of 18057 activity.

Gene ID 21617

The human 21617 sequence (SEQ ID NO:75), known also as retinol dehydrogenase 10, is approximately 3624 nucleotides long including untranslated regions. The coding sequence, located at about nucleic acids 339 to 1364 of SEQ ID NO:75, encodes an 341 amino acid protein (SEQ ID NO:76).

In a TaqMan panel, 21617 mRNA expression was increased 10 fold in pooled breast tumors relative to the pooled normal breast, 8 fold in pooled colon tumors relative to pooled normal colon and over 100 fold in pooled lung tumors relative to pooled normal lung. 21617 mRNA is expressed at moderately high levels in kidney, spinal cord and nerve.

In an oncology TaqMan panel, 21617 mRNA expression was increased in breast tumors, lung tumors, colon tumors and colon to liver metastases. 21617 mRNA was not detected in normal breast, normal lung samples and normal colon samples.

In an expanded colon tumor panel, 21617 mRNA was expressed at increased levels in colon tumors (7 to 23 fold) relative to normal colon samples.

In an expanded colon liver metastases panel, 21617 mRNA was expressed at increased levels in colon tumors (2 to 6 fold) relative to normal colon samples. 21617 mRNA expression was also increased in colon to liver metastases (2 to 20 fold) relative to normal liver samples.

In situ hybridization showed 21617 mRNA was detected in colon tumors, colon to liver metastases, breast tumors and lung tumors. 21617 mRNA was also detected in normal liver samples, but to a lesser extent than the colon to liver metastases. 21617 was not detected in normal colon, normal breast or normal lung samples.

21617 mRNA expression is increased in some colon, breast and lung tumors relative to the respective normal tissue. 21617 encodes retinol dehydrogenase 10 (Invest Ophthalmol Vis Sci 2002; 43(11):3365-72), a member of short-chain dehydrogenase/reductase, a heterogenous superfamily of enzymes. In humans, 37 members were identified that fall into three functional classes; enzymes involved in intermediary metabolism, enzymes participating in lipid hormone metabolism, and enzymes with unknown functions (Chemico-Biological Interactions 2001; 130-2:699-705). Some of these family members have been associated with increased cancer risk. Selective inhibition of 21617 may result in an inhibition and/or reduction in tumor growth.

Due to 21617 function and expression, modulators of 21617 activity would be useful in treating human cancers, including but not limited to breast, colon and lung cancers. 21617 polypeptides of the present invention are useful in screening for modulators of 21617 activity.

Gene ID 39228

The human 39228 sequence (SEQ ID NO:77), known also as similar to NADPH oxidoreductase homolog, is approximately 1808 nucleotides long including untranslated regions. The coding sequence, located at about nucleic acids 285 to 1418 of SEQ ID NO:77, encodes an 377 amino acid protein (SEQ ID NO:78).

As assessed by TaqMan analysis, 39228 mRNA expression was highest in HUVEC, heart (congestive heart failure), skeletal muscle, brain cortex and lung tumor tissue samples. 39228 mRNA expression was higher in ovarian, colon, and lung tumors (single sample pool of 3 tissue isolates) when compared to expression in normal ovarian, colon, and lung (single sample pool of 3 tissue samples).

Further TaqMan analysis on oncology tissue panels indicated that 39228 mRNA was expressed at increased levels in 1/6 breast, in 1/5 ovary, in 5/5 lung and in 6/6 colon tumors when compared to their respective normal tissue samples (3/3 breast, 2/2 ovary, 3/3 lung, 2/2 colon). In addition, 39228 mRNA was expressed at higher levels in proliferating HMVEC when compared to arrested HMVEC.

TaqMan analysis using an angiogenesis panel indicated that 39228 mRNA was highly expressed in fetal kidney (2/2) and in heart (1/1) tissues relative to adult kidney (1/1) and heart (1/1) tissue samples. TaqMan analysis using another angiogenesis panel indicated that 39228 mRNA was highly expressed in a skin tumor sample (1/1). 39228 mRNA was also expressed at higher levels in Wilm's tumor (5/5) and other kidney tumors (1/1) relative to the normal control (kidney (1/1)).

TaqMan analysis on the EC panels also demonstrated that 39228 mRNA was upregulated in proliferating lung HMVEC (4/4), cardiac HMVEC (3/3) or HUVEC (4/4) relative to their arrested controls (lung HMVEC (4/4), cardiac HMVEC (3/3) or HUVEC (4/4)).

TaqMan analysis of the Cox inhibitor study indicates that 39228 mRNA expression is increased by treatment of HUVEC with 40-100 mM NS398 (a Cox2 selective inhibitor) or Sulindac for 10 h relative to a vehicle (DMSO) control.

In situ hybridization experiments conducted on 39228 confirms the TaqMan data in that ovary tumors are positive for this gene. Colon tumors as well as colon metastases, breast tumors, and lung tumors are all positive. Normal colon tissue and normal ovary are negative. Normal lung and normal breast tissue are slightly positive as are these tissues when measured by TaqMan. Normal liver is also slightly positive.

39228 is most closely related to the leukotriene B4 12-hydroxydehydrogenase subfamily of zinc alcohol dehydrogenases. Antagonists of the leukotriene B4 receptor have been shown to inhibit proliferation and induce apoptosis in pancreatic cancer cells (Clin Cancer Res 2002, 8, 323242). 39228 was discovered using a Cox inhibitor paradigm, consistent with its homology to leukotriene metabolic enzymes. 39228 will inhibit proliferation and induce apoptosis by dysregulating leukotriene-like signaling.

Due to 39228 function and expression, modulators of 39228 activity would be useful in treating human cancers, including but not limited to breast, ovarian, colon and lung cancers, and tumor angiogenesis. 39228 polypeptides of the present invention are useful in screening for modulators of 39228 activity.

Gene ID 49928

The human 49928 sequence (SEQ ID NO:79), an uncharacterized protein, is approximately 5275 nucleotides long including untranslated regions. The coding sequence, located at about nucleic acids 37 to 2706 of SEQ ID NO:79, encodes an 889 amino acid protein (SEQ ID NO:80).

As assessed by TaqMan analysis on a tissue panel, 49928 mRNA is expressed at a 5-fold increased level in the primary ovary tumor pool, at a 5-fold increased level in the primary colon tumor pool, and at an 11-fold increased level in the primary lung tumor pool when compared to their respective normal tissue pools. Other samples with high 49928 mRNA expression include pancreas, erythroid, brain and kidney.

In an oncology TaqMan tissue panel, 49928 mRNA is expressed at 2-162 fold increased levels in 5/5 primary breast tumor samples vs. 3/3 normal breast tissue samples, at 2-11 fold increased levels in 5/5 primary lung tumor samples vs. 3/3 normal lung tissue samples and at 3-13 fold increased levels in 2/3 primary colon tumor samples vs. 2/3 normal colon tissue samples.

In an ovarian cell synchronization TaqMan panel, 49928 mRNA shows regulation throughout the cell cycle. At 12 hrs post-release from a G2 block with 0.5 ug/ul nocadazole, SKOV-3 cells are in GI and express 3-fold increased levels of 49928. At 12 hrs post-release from a G2 block with 0.5 ug/ul nocadazole, OVCAR4 cells are in GI and express 2-fold increased levels of 49928. At 12 hrs post-release from a Gi block with 0.2 uM mimosine, SKOV-3 cells are in G2 and express 2-fold decreased levels of 49928. Therefore, 49928 mRNA appears to be expressed at higher levels in G1/S vs. G2/M.

In an oncology xenograft cell line TaqMan panel, 49928 mRNA is expressed in a subset of cell lines, including T47D, ZR75, MCF-7 and NCIH67.

49928 is a protein of unknown function. 49928 has 40% amino acid identity to human 2-oxoglutarate dehydrogenase (D10523), also known as alpha-ketoglutarate dehydrogenase. The alpha-ketoglutarate dehydrogenase complex is a multienzyme complex consisting of 3 protein subunits, alpha-ketoglutarate dehydrogenase (Elk, or oxoglutarate dehydrogenase; OGDH, EC 1.2.4.2), dihydrolipoyl succinyltransferase (E2k, or DLST), and dihydrolipoyl dehydrogenase (E3). The complex catalyzes a key reaction in the Krebs tricarboxylic acid cycle, converting a-ketoglutarate to succinyl coenzyme A. With homology to oxoglutarate dehydrogenase, 49928 could contribute to energy production via the Krebs cycle.

49928 mRNA is expressed at increased levels in subset of breast, ovarian, lung and colon tumors. 49928 also shows cell cycle-dependent regulation and some homology to a subunit (E1) of the 2-oxoglutarate dehydrogenase multienzyme complex. Tumors may require increased levels of 49928 for cell-cycle regulated energy production. Inhibition of this enzyme may inhibit tumor growth.

Due to 49928 function and expression, modulators of 49928 activity would be useful in treating human cancers, including but not limited to breast, ovarian, colon and lung cancers. 49928 polypeptides of the present invention are useful in screening for modulators of 49928 activity.

Gene ID 54476

The human 54476 sequence (SEQ ID NO:81), known also as uncharacterized protein KIAA1290, is approximately 3622 nucleotides long including untranslated regions. The coding sequence, located at about nucleic acids 6 to 3038 of SEQ ID NO:81, encodes an 1010 amino acid protein (SEQ ID NO:82).

As assessed by TaqMan analysis, 54476 mRNA expression is restricted to a few tissues. Of the epithelial tumor pools on this panel, 54476 mRNA is expressed in the ovarian tumor pool. Other 54476 expressing tissue samples include brain, kidney and liver.

In an oncology tissue panel, 54476 mRNA is expressed at 32-122 fold increased levels in 4/5 primary ovary tumor samples vs. 2/2 normal ovary samples. Little or no expression was observed in breast, lung and colon tumors.

In an oncology xenograft cell line panel, 54476 mRNA is expressed in many tumor cell lines, including OVCAR3.

In an LRO synchronized cell panel, 54476 mRNA expression decreases 7-fold at 12 hours post-release from a mimosine block of the cell cycle, suggesting increased expression in G1.

In situ hybridization analysis of 54476 mRNA showed expression of this gene in ovarian carcinomas. No expression was seen in colon or lung tumors. Some expression was seen in infiltrating lymphocytes present in normal colon and colonic tumor samples. Normal liver and normal kidney showed some expression.

54476 is a protein of unknown function with 80% identity to 2-oxoglutarate decarboxylase, a subunit (E1) of the 2-oxoglutarate dehydrogenase multienzyme complex. The 2-oxoglutarate dehydrogenase complex (OGDHC) converts 2-oxoglutarate into succinyl-CoA as part of the citric acid cycle. The citric acid cycle is a central metabolic pathway into which all carbon compounds used by the cell will flow into and from which all biosynthetic needs of carbon compounds are satisfied. The cycle provides the complete oxidation of C2 units (acetyl-CoA) into carbon dioxide and gaining reductive power in the form of NADH and FADH₂. The cycle also provides metabolic intermediates for biosynthetic purposes of gluconeogenesis and amino acids and produces 1 GTP.

54476 expression in tumors is specific to ovarian cancers. Overexpression of 54476 in these cancers suggests a unique requirement for the activity of this enzyme in ovarian cancer. Inhibition of 54476 is predicted to inhibit the growth of ovarian cancers.

Due to 54476 function and expression, modulators of 54476 activity would be useful in treating human cancers, including but not limited to ovarian cancer. 54476 polypeptides of the present invention are useful in screening for modulators of 54476 activity.

Gene ID 62113

The human 62113 sequence (SEQ ID NO:83), a predicted acyl-CoA dehydrogenase of unknown function, is approximately 3030 nucleotides long including untranslated regions. The coding sequence, located at about nucleic acids 238 to 2580 of SEQ ID NO:83, encodes an 780 amino acid protein (SEQ ID NO:84).

As assessed by TaqMan analysis, 62113 mRNA is expressed in tumors of breast, ovary, lung and colon. Lung tumors specifically seem to express 62113 mRNA at increased levels and therefore may have an increased dependency on 62113 enzymatic function.

62113 is a predicted acyl-CoA dehydrogenase of unknown function. Acyl-CoA dehydrogenases [1,2,3] are enzymes that catalyze the alpha,beta-dehydrogenation of acyl-CoA esters and transfer electrons to ETF, the electron transfer protein. Acyl-CoA dehydrogenases are FAD flavoproteins. This family currently includes five eukaryotic isozymes that catalyze the first step of the beta-oxidation cycles for fatty acids with various chain lengths. These are short (SCAD) (EC 1.3.99.2), medium (MCAD) (EC 1.3.99.3), long (LCAD) (EC 1.3.99.13), very-long (VLCAD) and short/branched (SBCAD) chain acyl-CoA dehydrogenases. These enzymes are located in the mitochondrion. They are all homotetrameric proteins of about 400 amino acid residues except VLCAD which is a dimer and which contains, in its mature form, about 600 residues.

Overexpression of this gene may be linked to the increased energy requirements for rapidly growing and dividing tumor cells. Therefore, inhibition of this acyl-CoA dehydrogenase may inhibit tumor growth.

Due to 62113 function and expression, modulators of 62113 activity would be useful in treating human cancers, including but not limited to breast, ovarian, colon and lung cancers. 62113 polypeptides of the present invention are useful in screening for modulators of 62113 activity.

Gene ID 64316

The human 64316 sequence (SEQ ID NO:85), known also as NAD synthetase 1, is approximately 2404 nucleotides long including untranslated regions. The coding sequence, located at about nucleic acids 97 to 2217 of SEQ ID NO:85, encodes a 706 amino acid protein (SEQ ID NO:86).

As assessed by TaqMan analysis, 64316 mRNA expression was increased 3-fold in pooled colon tumors relative to pooled normal colons. 64316 mRNA expression was also elevated 2.5-fold in pooled lung tumors relative to pooled normal lung. Other tissues with high 64316 mRNA expression included human umbilical vein endothelial cells (HUVEC), brain cortex and erythroid cells.

Further TaqMan analysis on an oncology panel showed 64316 mRNA expression was increased in 2/6 breast tumors (3 to 4.5 fold) relative to 3 normal breast samples, in 4/6 lung tumors (2 to 3.5 fold) relative to 3 normal lung samples, in 3/5 colon tumors (2 to 6.5 fold) relative to 2 normal colon samples, and in 2/2 prostate tumors (2.5 fold) relative to 2 normal prostate samples.

TaqMan analysis on a colon metastases panel showed 64316 mRNA expression was increased in 3/4 colon tumors (2 to 2.5 fold) and in 8/16 colon to liver metastases (2 to 4 fold) relative to 3 normal colon and 3 normal liver samples.

TaqMan analysis on an expanded breast tumor panel showed 64316 mRNA expression was increased in 8/20 breast tumors (2 to 6 fold) relative to 2/4 normal breast samples. The high 64316 expression in 2/4 normal breast samples is suspected to be an artifact as these samples demonstrate high expression in almost all genes tested.

TaqMan analysis on an expanded lung tumor panel showed 64316 mRNA expression was increased in 8/33 lung tumors (2 to 3 fold) relative to 5 normal lungs and 1 normal lung trachea.

64316 expression was detected at low levels in 2/2 normal lungs by in situ hybridization. 64316 expression was detected at high levels in 0/2 lung squamous cell carcinomas, in 2/2 lung adenocarcinomas and in 2/2 poorly differentiated non-small cell carcinomas. 64316 was detected at low levels in 3/4 colon to liver metastases and at high levels in 1/4 colon to liver metastases.

64316 encodes NAD synthetase 1, which catalyzes the final metabolic step in NAD biosynthesis (Journal of Biological Chemistry; 278(13):10914-21 (2003)). The coenzyme NAD plays a major role in metabolism as it participates in the majority of cellular reductive/oxidative reactions. 64316 may make an ideal target for anti-cancer drug development, as depletion of NAD is known to result in cell death (Trends Pharmacol Sci; 20:171-81 (1999)). In addition, decreased NAD levels may increase p53-dependent apoptosis in response to DNA damage or oxidative stress as a result of reduced NAD-dependent p53 deacetylation induced by Sir2 (Cell; 107:137-148 (2001)). As depletion of NAD leads to cell death, selective inhibition of 64316 may result in decreased NAD, leading to inhibition and/or reduction of tumor growth.

Due to 64316 function and expression, modulators of 64316 activity would be useful in treating human cancers, including but not limited to breast, colon and lung cancers. 64316 polypeptides of the present invention are useful in screening for modulators of 64316 activity.

Gene ID 12264

The human 12264 sequence (SEQ ID NO:87), known also as kinesin-like protein KIF2 (Kinesin-2) (HK2), is approximately 2905 nucleotides long including untranslated regions. The coding sequence, located at about nucleic acids 19 to 2058 of SEQ ID NO:87, encodes a 679 amino acid protein (SEQ ID NO:88).

As assessed by TaqMan analysis, 12264 mRNA was expressed at higher levels in colon and lung tumors (single sample pool of 3 tumors) with respect to normal colon and lung (single sample pool of 3 tissue samples). 12264 mRNA was also highly expressed in human umbilical vein endothelial cells (HUVEC), brain cortex, hypothalamus and megakaryocytes (single sample pool of 3 tissue samples).

Further TaqMan analysis on an oncology tissue panel indicated that 12264 mRNA was expressed at increased levels in 1/6 breast, in 6/6 lung, and in 1/5 colon tumors when compared to their respective normal tissue samples (3/3 breast, 3/3 lung, 3/3 colon). 12264 mRNA was expressed at increased levels in proliferating human microvascular endothelial cells (HMVEC) relative to arrested HMVEC.

TaqMan analysis of an angiogenesis panel (fetal vs. adult tissues) indicated that 12264 mRNA was expressed at increased levels in fetal liver (2/2), in adrenal (2/3), in kidney (2/2) and in umbilical cord (2/2) relative to adult angiogenic tissue samples (23/24). 12264 mRNA was also expressed at increased levels in uterine and endometrial adenocarcinomas (5/9). TaqMan analysis using another angiogenesis panel (hemangiomas and necrotic and angiogenic tumors) indicated that 12264 mRNA was expressed at higher levels in 2/8 hemangiomas relative to 1/1 normal skin tissue samples. 12264 mRNA was also expressed at higher levels in necrotic tumors (1/4 ovary, 3/3 colon, and 3/3 lung tumors) and in Wilm's tumor relative to normal controls (ovary (1/1), colon (1/1), lung (1/1), and kidney (1/1)).

In an expanded lung tumor panel, TaqMan analysis indicated that 12264 mRNA was increased in 14/32 lung tumors when compared to 6/6 normal lung tissue samples.

In a colon ACA tumor panel, TaqMan analysis indicated that 12264 mRNA was increased in 6/15 colon primary tumors when compared to 5/6 normal colon tissue samples and in 3/5 colon tumor metastasis samples when compared to either 6/6 normal colon tissue samples or 6/6 normal liver tissue samples.

In a colon tumor metastasis panel, TaqMan analysis indicated that 12264 mRNA was increased in 4/16 samples of colon tumor metastasis to the liver when compared to either 3/3 normal colon tissue samples or 3/3 normal liver tissue samples. 12264 mRNA expression was also increased in 6/14 samples of colon tumor metastasis to other sites when compared to 3/3 normal colon tissue samples.

TaqMan analysis of 12264 mRNA expression in an “endothelial cell in vitro proliferation, arrest and tube formation” panel indicated increased expression in proliferating HUVEC (1/1) and lung HMVEC (3/3) when compared to arrested HUVEC (1/1) and lung HMVEC (3/3).

In situ experiments conducted on 12264 indicated expression of 12264 in primary colon and lung tumors as well as in a number of fetal tissues and Wilhm's tumor. Most tumor foci in each tumor sample are positive for expression. Very little stromal expression was seen. Normal colon tissue showed some low level expression in epithelial cells, with some background in infiltrating lymphocytes. Normal lung tissue was mostly negative.

The Kin I subfamily of kinesins are microtubule depolymerases. MCAK is a kinesin important to anaphase progression and localizes to the centromere. KIF2 localizes to the lysosome. It appears to be involved in protein trafficking, e.g., KIF2 suppression by antisense treatment results in a dramatic accumulation of betagc, a beta subunit of the IGF-1 receptor, within the cell body and its complete disappearance from growth cones. KIF2 suppression produces a dramatic inhibition of neurite outgrowth (J Cell Biol, 138:657-69 (1997)). Taxol affects protein trafficking within the cell and these effects may be important to its efficacy. KIF2 inhibition would prevent protein trafficking by interfering with microtubule dynamics associated with lysosomal/endosomal function. The function of key receptors and secreted proteins would thereby be prevented.

Due to 12264 function and expression, modulators of 12264 activity would be useful in treating human cancers, including but not limited to breast, colon and ovarian cancers, and tumor angiogenesis. 12264 polypeptides of the present invention are useful in screening for modulators of 12264 activity.

Gene ID 32362

The human 32362 sequence (SEQ ID NO:89), known also as serine/threonine protein kinase SAK, is approximately 3092 nucleotides long including untranslated regions. The coding sequence, located at about nucleic acids 141 to 3053 of SEQ ID NO:89, encodes a 970 amino acid protein (SEQ ID NO:90).

Transcriptional profiling identified increased expression of 32362 mRNA in a subset of lung tumors when compared to normal lung samples.

TaqMan analysis demonstrated that 32362 mRNA expression was restricted in normal tissues. 32362 mRNA expression was upregulated in breast (1/6), in ovarian (4/5), in colon (4/4), and in lung (3/5) tumors as compared to their respective normal controls. The trend of increased tumor expression (albeit at low levels) was confirmed in TaqMan analyses of an expanded lung panel (12/33 primary tumors) and an expanded breast panel (8/15 primary tumors).

In situ hybridization showed low 32362 mRNA expression in normal lung and ovarian samples with increased levels of expression in lung and ovarian tumors.

SAK is a member of the polo family of kinases. As with its better-known homologue PLK1, SAK function has been implicated in the progression of cells through mitosis (Proc Natl Acad Sci USA., 91:6388-92 (1994)). Knockout of this gene in mice results in embryonic day 7.5 lethality, characterized by a marked increase in mitotic and apoptotic cells in the embryo (Curr Biol., 11:441-6 (2001)). Increased expression of SAK in tumors may facilitate proliferation by enabling progression of the cells through mitosis.

Due to 32362 function and expression, modulators of 32362 activity would be useful in treating human cancers, including but not limited to breast, lung and ovarian cancers. 32362 polypeptides of the present invention are useful in screening for modulators of 32362 activity.

Gene ID 58198

The human 58198 sequence (SEQ ID NO:91), a novel tetrahydrofolate dehydrogenase/cyclohydrolase, is approximately 3164 nucleotides long including untranslated regions. The incomplete coding sequence, located at about nucleic acids 3 to 2756 of SEQ ID NO:91, encodes a 917 amino acid protein (SEQ ID NO:92).

As assessed by TaqMan analysis, 58198 mRNA expression was increased approximately 300-fold in pooled colon tumors relative to pooled normal colons. 58198 mRNA expression was also elevated 5-fold in pooled lung tumors relative to pooled normal lung and 2.5-fold in pooled breast tumors relative to pooled normal breast. Other tissues expressing high 58198 mRNA levels included human umbilical vein endothelial cells (HUVEC), normal artery, coronary smooth muscle cells, primary osteoblasts, and normal brain cortex.

In an oncology TaqMan panel, 58198 mRNA expression was increased 2.5 to 7-fold in breast tumors relative to normal breast samples, 2 to 4.5-fold in lung tumors relative to normal lung samples, and 3 to 7.5-fold in colon tumors relative to normal colon samples. 58198 mRNA was expressed at high levels in the human colorectal carcinoma cell line HCT116.

In an expanded colon tumor panel, 58198 mRNA expression was increased 3 to 15-fold in colon tumors and 4 to 31-fold in colon to liver metastases. 58198 mRNA was expressed at very low levels in normal liver.

In a colon metastasis panel, 58198 mRNA expression was increased 7.5 to 19-fold in colon tumors, 3 to 50-fold in colon to liver metastases and 2 to 31-fold in colon metastases to tissues other than the liver. 58198 mRNA was expressed at low levels in normal liver.

In an expanded lung tumor panel, one normal lung sample expressed moderately high 58198 mRNA levels. Omitting that sample, 58198 mRNA expression was increased 2 to 31-fold in 13/33 lung tumors relative to normal lung and normal lung trachea.

In an expanded breast tumor panel, 58198 mRNA expression was increased 2.5 to 6-fold in breast tumors and 5-fold in a breast tumor metastases when compared to normal breast tissue samples.

58198 mRNA expression is increased in some colon, lung and breast tumors. 58198 encodes a novel methylenetetrahydrofolate dehydrogenase/methenyl cyclohydrogenase. This family of enzymes participates in folate-mediated one-carbon metabolism, which is essential to nucleic acid biosynthesis, mitochondrial protein biosynthesis, amino acid biosynthesis and vitamin metabolism (Biochemistry, 29:7089-7094 (1990); Biochem J, 350:609-29 (2000); MCB, 22(12): 4158-66 (2002)). As 58198 expression is increased in some cancers, and because it plays a role in several essential biosynthetic processes associated with cell growth regulation, it may make an ideal target for anti-cancer drug design. Selective inhibition of 58198 may result in inhibition and/or reduction of tumor growth.

Due to 58198 function and expression, modulators of 58198 activity would be useful in treating human cancers, including but not limited to breast, colon and lung cancers. 58198 polypeptides of the present invention are useful in screening for modulators of 58198 activity.

Gene ID 2887

The human 2887 sequence (SEQ ID NO:93), known also as glutamate dehydrogenase 1, is approximately 2970 nucleotides long including untranslated regions. The coding sequence, located at about nucleic acids 14 to 1690 of SEQ ID NO:93, encodes a 558 amino acid protein (SEQ ID NO:94).

As assessed by TaqMan analysis, 2887 mRNA expression was broad in normal tissues, with the highest expression observed in CNS tissues. 2887 mRNA showed limited upregulated expression in breast (3/6), in lung (1/6) and in colon tumors (2/5) when compared to their respective normal tissues.

In situ hybridization experiments consucted on 2887 indicated that 2887 is expressed in primary colon (3/4), in lung (3/3), and in ovarian (4/5) carcinomas. Normal tissue showed little or no expression. Lung and ovarian tumors showed the highest and most homogeneous expression.

Expression of 2887 mRNA in RNAi transfected cells was >20% of that seen in mock transfected controls.

Treatment of tumor cells with RNAi directed against 2887 mRNA results in varying levels of growth inhibition (72 hours post-transfection) in the following cell lines:

TABLE 1

| Cell Line | % Growth Inhibition |
|---|---|
| A549 | 44% (1 of 2 assays) |
| HCT116 | 35% (average, 2 of 3 assays) |
| HT29 | 43% (one assay) |

Reduction of 2887 mRNA expression in tumor cells by transfection with siRNA oligos specific for that gene results in cell growth inhibition. RNAi studies in three different cell lines (A549, HT29, and HCT116) showed somewhat consistent growth inhibition over a 72-hour period. Small molecule inhibitors of 2887 would be expected to have a similar effect on tumor cell growth, and are therefore attractive cancer therapeutic candidates.

Due to 2887 function and expression, modulators of 2887 activity would be useful in treating human cancers, including but not limited to breast, colon and lung cancers. 2887 polypeptides of the present invention are useful in screening for modulators of 2887 activity.

Gene ID 3205

The human 3205 sequence (SEQ ID NO:95), known also as arginine N-methyltransferase 2, is approximately 2096 nucleotides long including untranslated regions. The coding sequence, located at about nucleic acids 177 to 1478 of SEQ ID NO:95, encodes a 433 amino acid protein (SEQ ID NO:96).

As assessed by TaqMan analysis, 3205 mRNA expression was broad in normal tissues, with the highest expression observed in CNS tissues. 3205 mRNA expression was upregulated in lung tumors (5/6) when compared to normal lung tissues.

Expression of 3205 mRNA in RNAi transfected cells was 20% of that seen in mock transfected controls.

Treatment of tumor cells with RNAi directed against 3205 mRNA results in varying levels of growth inhibition (72 hours post-transfection) in the following cell lines:

TABLE 2

| Cell Line | % Growth Inhibition |
|---|---|
| A549 | 62% (average, 2 separate assays) |
| HCT116 | 51% (average, 3 separate assays) |
| HT29 | 58% (one assay) |

Reduction of 3205 mRNA expression in tumor cells by transfection with siRNA oligos specific for that gene results in cell growth inhibition. RNAi studies in three different cell lines (A549, HT29, and HCT116) showed levels of growth inhibition upon 3205 mRNA expression reduction comparable to those seen in the PLK1 positive control. Small molecule inhibitors of 3205 would be expected to have a similar effect on tumor cell growth, and are therefore attractive cancer therapeutic candidates.

Due to 3205 function and expression, modulators of 3205 activity would be useful in treating human cancers, including but not limited to lung cancer. 3205 polypeptides of the present invention are useful in screening for modulators of 3205 activity.

Gene ID 8557

The human 8557 sequence (SEQ ID NO:97), known also as aldose reductase, is approximately 1331 nucleotides long including untranslated regions. The coding sequence, located at about nucleic acids 10 to 960 of SEQ ID NO:97, encodes a 316 amino acid protein (SEQ ID NO:98).

As assessed by TaqMan analysis, 8557 mRNA expression was broad in normal tissues, with the highest expression observed in adrenal gland and kidney. 8557 mRNA expression was upregulated in lung (3/6) and in breast tumors (2/6) when compared to their respective normal tissues.

In situ hybridization experiments conducted on 8557 indicated that 8557 mRNA is expressed in primary breast (4/5) and lung tumors (5/5). Lung tumors showed greater and more homogeneous expression as compared to breast tumors. No expression was seen in normal breast or lung tissue.

Expression of 8557 mRNA in RNAi transfected cells was >10% of that seen in mock transfected controls.

Treatment of tumor cells with RNAi directed against 8557 results in varying levels of growth inhibition (72 hours post-transfection) in the following cell lines:

TABLE 3
Example Bottle Neck and Tolerances for use with Closure Capsules

| Station | Nominal Diameter | Tolerance |
|---|---|---|
| A | 1.294* inches | +/−0.031 inches |
| B | D - 0.088 inches | +/−0.020 inches |
| C | D - 0.160 inches | +/−0.020 inches |
| D | D - 0.120 inches | +0.013/−0.012 inches |

*OR any desirable bottleneck diameter

Reduction of 8557 mRNA expression in tumor cells by transfection with siRNA oligos specific for that gene results in cell growth inhibition. RNAi studies in three different cell lines (A549, HT29, and HCT116) showed consistent growth inhibition over a 72-hour period. Small molecule inhibitors of 8557 would be expected to have a similar effect on tumor cell growth, and are therefore attractive cancer therapeutic candidates.

Due to 8557 function and expression, modulators of 8557 activity would be useful in treating human cancers, including but not limited to breast and lung cancers. 8557 polypeptides of the present invention are useful in screening for modulators of 8557 activity.

Gene ID 9600

The human 9600 sequence (SEQ ID NO:99), known also as 7,8-dihydro-8-oxoguanine triphosphatase (8-oxo-dGTPase), is approximately 772 nucleotides long including untranslated regions. The coding sequence, located at about nucleic acids 30 to 623 of SEQ ID NO:99, encodes a 197 amino acid protein (SEQ ID NO:100).

As assessed by TaqMan analysis, 9600 mRNA is expressed at a 15-fold increased level in the colon tumor pool vs. the normal colon tissue pool, and at a 6-fold increased level in the lung tumor pool vs. the normal lung tissue pool. Other samples expressing 9600 mRNA include erythroid cells, human umbilical vein endothelial cells (HUVEC), megakaryocytes and primary osteoblasts (each of these samples has been cultured in vitro).

In an oncology TaqMan panel, 9600 mRNA is expressed at increased levels (5-62 fold) in primary lung tumors when compared to normal lung tissue samples. 9600 mRNA is expressed at increased levels (12-308 fold) in primary breast tumors and at increased levels (3-16 fold) in primary colon tumor samples. 9600 mRNA is expressed at 58-fold increased level in a pooled colon to liver metastases sample when compared to a normal liver sample.

In an expanded lung cancer tissue panel, 9600 mRNA is expressed at increased levels (2-40 fold) in primary lung tumors when compared to normal lung tissue samples.

In an expanded colon cancer tissue panel, 9600 mRNA is not expressed (Cts>33) in normal liver samples, but is expressed (Cts<28) in colon metastases to the liver.

In an oncology xenograft cancer cell line panel, 9600 mRNA is expressed at variable levels in all tumor cell lines. Some cell lines express very high levels (Cts=22), including MCF-7, NCI-H322 and NCI-H69.

In a breast cancer cell model panel, 9600 mRNA is expressed at a 2.5-fold increased level in transformed, activated H-ras-expressing MCF10AT1 cells vs. normal MCF10A cells, and at a 2-fold increased level in transformed, activated H-ras-expressing MCF10AT3B cells vs. normal MCF10A cells.

In a lung cancer cell model panel, 9600 mRNA expression is reduced 3-fold in H125 cells expressing a p53:ERhb protein and induced for p53 activity with 4-hydroxytamoxifen.

One form of DNA damage caused by oxygen radicals is the oxidation of guanine base (8-oxoguanine). The catalytic activity of 8-oxo-dGTPase (NUDT1 or MTH1) is: 8-oxo-dGTP+H2O=8-oxo-dGMP+diphosphate. It also acts on other purine nucleoside triphosphates. 8-oxo-dGTPase is responsible for preventing misincorporation of 8-oxo-dGTP into DNA, thus preventing A:T to C:G transversions and protecting genetic information from the deleterious effects of oxygen radicals. The highest expression levels are seen in thymus, testis, embryo and PBLs.

9600 mRNA is expressed at increased levels in a subset of epithelial tumors, notably primary lung tumors and colon cancer metastases. Rapidly proliferating tissues require increased capacity for maintaining genome integrity. Increased expression of 8-oxo-dGTPase may serve to protect tumor cells against DNA damaging effects of oxygen radicals. Inhibiting the 8-oxo-dGTPase activity of tumor cells may result in irreparable DNA damage and cell death.

Due to 9600 function and expression, modulators of 9600 activity would be useful in treating human cancers, including but not limited to breast, colon and lung cancers. 9600 polypeptides of the present invention are useful in screening for modulators of 9600 activity.

Gene ID 9693

The human 9693 sequence (SEQ ID NO:101), known also as chlordecone reductase, is approximately 972 nucleotides long including untranslated regions. The coding sequence, located at about nucleic acids 1 to 972 of SEQ ID NO:101, encodes a 323 amino acid protein (SEQ ID NO:102).

As assessed by TaqMan analysis, 9693 mRNA expression was broad in normal tissues, with the highest expression in skeletal muscle and liver. Increased 9693 mRNA expression was observed in breast (1/6) and lung (2/6) tumors when compared to their respective normal tissues. 9693 mRNA was upregulated in a pool of 3 lung tumors versus lung normal tissue.

In an oncology xenograft cancer cell line panel, 9693 mRNA is expressed at variable levels in a subset of tumor cell lines. Some cell lines express very high levels (Cts=22), including MCF-7, NCI-H322, NCI-H460, and A549.

In situ hybridization experiments conducted on 9693 indicated that 9693 mRNA was highly over-expressed in lung carcinoma (3/5); moreover, this target appears to be more specific to squamous cell carcinomas as compared to adenocarcinoma. Both the ISH expression data and clinical TaqMan data indicate preferential expression in squamous cell histology. Clinical TaqMan data indicated that 9693 is a lung cancer target and this was confirmed by ISH.

Expression of 9693 mRNA in RNAi transfected cells was >20% of that seen in mock transfected controls.

Treatment of tumor cells with RNAi directed against 9693 results in varying levels of growth inhibition (72 hours post-transfection) in the following cell lines:

TABLE 4

| Cell Line | % Growth Inhibition |
|---|---|
| A549 | 36% (1 of 2 assays) |
| HCT116 | 44% (average, 3 of 3 assays) |
| HT29 | 43% (one assay) |

Reduction of 9693 mRNA expression in tumor cells by transfection with siRNA oligos specific for that gene results in cell growth inhibition. RNAi studies in three different cell lines (A549, HT29, and HCT116) showed somewhat consistent growth inhibition over a 72-hour period. Small molecule inhibitors of 9693 would be expected to have a similar effect on tumor cell growth, and are therefore attractive cancer therapeutic candidates.

Due to 9693 function and expression, modulators of 9693 activity would be useful in treating human cancers, including but not limited to lung cancer. 9693 polypeptides of the present invention are useful in screening for modulators of 9693 activity.

Gene ID 44867

The human 44867 sequence (SEQ ID NO:103), which is similar to Bmp2-inducible kinase, is approximately 2770 nucleotides long including untranslated regions. The partial coding sequence, located at about nucleic acids 2 to 2563 of SEQ ID NO:103, encodes a 853 amino acid protein (SEQ ID NO:104).

As assessed by TaqMan analysis, 44867 mRNA expression was highest in erythroid cells, megakaryocytes, and CNS tissues. 44867 mRNA had lower expression in heart and pancreas. There was no expression in other tissues tested. 44867 mRNA expression was upregulated in lung, breast and colon tumors on a clinical panel as compared to their respective normal tissues. 44867 mRNA expression was also upregulated in lung tumor samples as compared to normal lung tissue samples on an expanded TaqMan panel. There was decreased 44867 mRNA expression in NCI-H125 cells upon activation of a p53ER fusion protein.

In situ hybridization (ISH) showed low 44867 mRNA expression in normal lung samples and lung tumors tested. There was no expression in normal colon, with epithelial expression in colon tumor samples. There was also low 44867 mRNA expression in normal breast and breast tumor samples.

44867 is the human homologue of the mouse BMP-2 inducible kinase, a recently identified protein that has been shown to attenuate osteoblast differentiation (J. Biol. Chem. 276(45): 42213-42218 (2001)). While not wanting to be bound by theory, it is hypothesized that 44867 expression in tumor cells may play a role in locking them into an immature state, enabling continued proliferation in environments where the cells would otherwise terminally differentiate.

Due to 44867 function and expression, modulators of 44867 activity would be useful in treating human cancers, including but not limited to breast, colon and lung cancers. 44867 polypeptides of the present invention are useful in screening for modulators of 44867 activity.

Gene ID 53058

The human 53058 sequence (SEQ ID NO:105), known also as BMK1 alpha kinase, MAP kinase 7 and Erk5, is approximately 2980 nucleotides long including untranslated regions. The coding sequence, located at about nucleic acids 222 to 2672 of SEQ ID NO:105, encodes a 816 amino acid protein (SEQ ID NO:106).

As assessed by TaqMan analysis, 53058 mRNA expression was generally low in normal tissues, with the highest expression in CNS tissue and normal artery. Further TaqMan analyses using an oncology panel demonstrated that 53058 mRNA expression increased in lung tumors (3/6) and in breast tumors (2/6) when compared to their respective normal tissues.

In situ hybridization experiments conducted on 53058 showed very low 53058 mRNA expression in 2 normal lung samples and low to moderate expression in 4/5 lung tumors tested. There was no 53058 mRNA expression in normal breast (1/1), with low to moderate expression in 3/3 breast tumor samples.

53058 is the human ERK5 gene, a MAP kinase family member that has been implicated in downstream signaling of the ErbB receptor and raf oncogenes (Mol. Cell. Biol. 22(1):270-85 (2002); J. Biol. Chem. 274(44):31588-92(1999)), as well as in the NF-kappaB pathway (J. Biol. Chem. 276(11):7927-31 (2001)). High expression of Erk5 in tumors may increase the proliferative and survival capacity of tumor cells.

Due to 53058 function and expression, modulators of 53058 activity would be useful in treating human cancers, including but not limited to breast and lung cancers. 53058 polypeptides of the present invention are useful in screening for modulators of 53058 activity.

Gene ID 55556

The human 55556 sequence (SEQ ID NO:107), known also as serine/threonine-protein kinase PAK 6 (p21-activated kinase 6) (PAK-6), is approximately 2669 nucleotides long including untranslated regions. The coding sequence, located at about nucleic acids 199 to 2244 of SEQ ID NO:107, encodes a 681 amino acid protein (SEQ ID NO:108).

As assessed by TaqMan analysis, 55556 mRNA had restricted tissue expression: the highest expression was in CNS tissues, with lower expression in pancreas and kidney. There was significant upregulation of 55556 mRNA expression in tumors of the breast, ovary, lung and colon when compared to their respective normal tissues. There was also increased expression in lung tumors as compared to normal lung samples on an expanded lung tumor TaqMan panel.

55556 is human PAK6, a member of the PAK kinase family that is distinguished by binding domains for the cdc42 and rac small G proteins (J. Int. J. Biochem. Cell. Biol. 34(7):713-7 (2002)). While the function of PAK6 has not been well characterized, recent studies suggest that it can bind to and modulate the transcriptional activity of steroid hormone receptors (Mol. Endocrinol. 16(1):85-99 (2002)).

While not wanting to be bound by theory, it is hypothesized that high expression of PAK6 in tumor cells alters their proliferative and metastatic potential by altering the transcriptional environment in these cells.

Due to 55556 function and expression, modulators of 55556 activity would be useful in treating human cancers, including but not limited to breast, colon, lung and ovarian cancers. 55556 polypeptides of the present invention are useful in screening for modulators of 55556 activity.

Gene ID 57658

The human 57658 sequence (SEQ ID NO:109), known also as uridine-cytidine kinase 1 (UCK 1), is approximately 2160 nucleotides long including untranslated regions. The coding sequence, located at about nucleic acids 95 to 928 of SEQ ID NO:109, encodes a 277 amino acid protein (SEQ ID NO:110).

As assessed by TaqMan analysis, 57658 mRNA expression was broad in normal tissues, with the highest expression in CNS tissues and erythroid cells. Further TaqMan experiments conducted on an oncology tissue panel demonstrated that 57658 was upregulated in 2/6 breast tumors and in 5/6 lung tumors when compared to their respective normal tissues.

In an oncology xenograft cancer cell line panel, 57658 mRNA was expressed at variable levels in all tumor cell lines. Some cell lines express very high levels (Cts=24), including MCF-7, NCI-H69 and 293.

In situ experiment conducted on 57658 indicated that 57658 mRNA is expressed in primary lung (4/5), colon (4/4), and ovarian (2/3) tumors. Although some normal tissues are positive, the expression levels are much lower than that seen in the tumor counterpart. The clinical TaqMan data indicated that 57658 is regulated in breast and lung tumors, whereas the in situ data showed more regulation between normal ovarian tissue and ovarian tumor as compared to the TaqMan data. 57658 appears to be primarily a lung and ovarian target based on regulation and expression patterns.

Expression of 57658 mRNA in RNAi transfected cells was ˜50% of that in mock transfected controls.

Treatment of tumor cells with siRNA directed against 57658 mRNA results in growth inhibition (72 hours post-transfection) in the following cell lines:

TABLE 5

| Cell Line | % Growth Inhibition |
|---|---|
| HCT116 | 47.8% (average, 2 of 3 assays) |

Reduction of 57658 mRNA expression in tumor cells by transfection with siRNA oligos specific for that gene results in cell growth inhibition. RNAi studies in HCT116 cells showed levels of growth inhibition comparable to the PLK1 positive control. Small molecule inhibitors of 57658 would be expected to have a similar effect on tumor cell growth, and are therefore attractive cancer therapeutic candidates.

Due to 57658 function and expression, modulators of 57658 activity would be useful in treating human cancers, including but not limited to breast and lung cancers. 57658 polypeptides of the present invention are useful in screening for modulators of 57658 activity.

Gene ID 2208

The human 2208 sequence (SEQ ID NO:111), known also as PTEN induced putative kinase 1, is approximately 2162 nucleotides long including untranslated regions. The coding sequence, located at about nucleic acids 76 to 1821 of SEQ ID NO:111, encodes a 581 amino acid protein (SEQ ID NO:112).

As assess by TaqMan analysis, 2208 mRNA showed broad normal tissue expression with the highest expression in CNS tissues. Further TaqMan experiments conducted on an oncology tissue panel demonstrated that 2208 mRNA expression was upregulated in breast and lung tumors when compared to their respective normal tissues.

Expression of 2208 mRNA in RNAi transfected cells was <5% of that seen in mock transfected controls.

Treatment of tumor cells with RNAi directed against 2208 mRNA results in varying levels of growth inhibition (72 hours post-transfection) in the following cell lines:

TABLE 6

| Cell Line | % Growth Inhibition |
|---|---|
| A549 | 37% (1 of 2 assays) |
| HCT116 | 46.4% (average, 2 of 3 assays) |
| HT29 | no inhibition |

Reduction of 2208 mRNA expression in tumor cells by transfection with siRNA oligos specific for that gene results in cell growth inhibition. RNAi studies in two different cell lines (A549 and HCT116) showed significant levels of growth inhibition coincident with reduction of 2208 mRNA expression. Small molecule inhibitors of 2208 would be expected to have a similar effect on tumor cell growth, and are therefore attractive cancer therapeutic candidates.

Due to 2208 function and expression, modulators of 2208 activity would be useful in treating human cancers, including but not limited to breast and lung cancers. 2208 polypeptides of the present invention are useful in screening for modulators of 2208 activity.

Gene ID 10252

The human 10252 sequence (SEQ ID NO:113), known also as Pyridoxine (or Pyridoxal) kinase, is approximately 960 nucleotides long including untranslated regions. The coding sequence, located at about nucleic acids 7 to 945 of SEQ ID NO:113, encodes a 312 amino acid protein (SEQ ID NO:114).

As assessed by TaqMan analysis, 10252 mRNA expression was increased approximately 10 fold in the pooled colon tumors relative to the pooled normal colons and 2.5 fold in pooled lung tumors relative to pooled normal lung. Other tissues expressing high 10252 mRNA levels included human umbilical vein endothelial cells (HUVEC), coronary smooth muscle cells, normal kidney, normal brain cortex and normal brain hypothalamus.

In an oncology TaqMan panel, 10252 mRNA expression was increased 4.5 and 5.5-fold in 3/5 breast tumors, 2-fold in 3/6 lung tumors, and 3-fold in 1/1 pooled colon to liver metastases relative to their respective normal controls. 10252 mRNA expression was also increased in 1/6 ovarian tumors when compared to normal ovarian tissue. 10252 mRNA was expressed at high levels in the human colorectal carcinoma cell line HCT116.

In an SVA colon polyp panel, 10252 mRNA expression was increased 3.5-fold in hyperplastic polyps, 2 to 3-fold in adenomas, and 2 to 4-fold in adenocarcinomas relative to normal colon samples.

In an expanded colon tumor panel, 10252 mRNA was expressed at increased levels in colon tumors and colon to liver metastases relative to normal colon samples.

In a colon metastases panel, 10252 mRNA expression was increased in colon tumors, colon to liver metastases and colon metastases to tissues other than the liver relative to normal colon samples. 10252 mRNA was expressed at low levels in normal liver.

In an expanded lung tumor panel, 10252 mRNA expression was increased in lung tumors relative to normal lungs and normal lung trachea.

In situ hybridization experiments conducted on 10252 indicated that primary lung (4/4) and colon (4/4) tumors are positive for the target as well as colon metastasis to the liver (3/3). The normal lung and colon tissue are slightly positive (above background), but are much lower and heterogeneous in expression.

RNAi studies in HCT116 and A549 cells showed variable levels of growth inhibition coincident with reduction of 10252 expression.

10252 mRNA expression is increased in some colon and lung tumors. 10252 encodes a pyridoxine kinase. Pyridoxine kinase catalyzes the phosphorylation of vitamin B6 to its active form, pyridoxal 5′-phosphate (Journal of Biochemistry, 180(7):1814-21(1998); Mol Cells, 10(4):452-9 (2000)). Although a significant amount of data exists demonstrating that pharmacologic levels of vitamin B6 supplementation reduce tumor cell proliferation, little has been reported on the dependency of tumors on physiologic levels. One report demonstrated that several human tumors display a significant dependence on vitamin B6 for proliferation (Anticancer Research, 8(4):813-8 (1988)). Tumors expressing increased levels of 10252 may be dependent on its activity, making it an ideal target for anti-cancer drug design. Selective inhibition of 10252 may result in inhibition and/or reduction of tumor growth.

Due to 10252 function and expression, modulators of 10252 activity would be useful in treating human cancers, including but not limited to colon and lung cancers. 10252 polypeptides of the present invention are useful in screening for modulators of 10252 activity.

Gene ID 10302

The human 10302 sequence (SEQ ID NO:115), known also as serum paraoxonase/arylesterase 3 (PON 3), is approximately 1075 nucleotides long including untranslated regions. The coding sequence, located at about nucleic acids 1 to 1065 of SEQ ID NO:115, encodes a 354 amino acid protein (SEQ ID NO:116).

As assessed by TaqMan analysis, 10302 mRNA expression was upregulated in tumors of the breast (1/6), lung (2/6) and colon (1/5) when compared to their respective normal tissues. On an expanded lung tumor panel (largely adenocarcinomas), 10302 mRNA showed increased expression in 5/20 lung tumor samples when compared to normal lung tissues. On an expanded colon tumor panel, 10302 mRNA showed increased expression in 5/14 colon tumor samples when compared to normal colon tissue samples. 10302 mRNA showed increased expression in a Beas2B cell line (K10) transformed with mutant kras.

In situ hybridization experiments on 10302 indicated that 10302 is expressed in primary lung (4/5) and colon (2/3) tumors, as well as in colon metastasis to the liver (2/3). Normal tissues show little to no expression of the target.

10302 is the human PON3 gene, a member of the paraoxonase family that has been shown to mitigate cellular damage caused by oxidative stress. 10302 appears to be upregulated in cells by expression of mutant kras. Transformation of 3T3 cells with kras has been shown to increase their resistance to oxidative stress by 10-fold (Biochem Biophys Res Commun. 229(3):739-45 (1996)).

Expression levels of 10302 are increased in tumor cells containing a constitutively active kras. This in turn allows increased tumor survival under conditions of oxidative stress.

Due to 10302 function and expression, modulators of 10302 activity would be useful in treating human cancers, including but not limited to breast, colon and lung cancers. 10302 polypeptides of the present invention are useful in screening for modulators of 10302 activity.

Gene ID 14218

The human 14218 sequence (SEQ ID NO:117), known also as serine/threonine kinase 17B or DAP kinase-related apoptosis-inducing protein kinase 2, is approximately 1627 nucleotides long including untranslated regions. The coding sequence, located at about nucleic acids 262 to 1380 of SEQ ID NO:117, encodes a 372 amino acid protein (SEQ ID NO:118).

As assessed by TaqMan analysis, 14218 mRNA showed broad tissue expression, with the highest expression in normal tonsil, lymph node, and osteoblasts. 14218 mRNA expression was upregulated in breast tumors and ovarian tumors when compared to their respective normal tissues.

Expression of 14218 mRNA in RNAi transfected cells was <35% of that seen in mock transfected controls.

Treatment of tumor cells with RNAi directed against 14218 mRNA results in varying levels of growth inhibition (72 hours post-transfection) in the following cell lines:

TABLE 7

| Cell Line | % Growth Inhibition |
|---|---|
| A549 | 42.9% (average, 2 assays) |

Reduction of 14218 mRNA expression in tumor cells by transfection with siRNA oligos specific for that gene results in cell growth inhibition. RNAi studies in A549 cells showed significant levels of growth inhibition coincident with reduction of 14218 expression. Small molecule inhibitors of 14218 would be expected to have a similar effect on tumor cell growth, and are therefore attractive cancer therapeutic candidates.

Due to 14218 function and expression, modulators of 14218 activity would be useful in treating human cancers, including but not limited to breast and ovarian cancers. 14218 polypeptides of the present invention are useful in screening for modulators of 14218 activity.

Gene ID 33877

The human 33877 sequence (SEQ ID NO:119), a glycosyl transferase, is approximately 2493 nucleotides long including untranslated regions. The coding sequence, located at about nucleic acids 402 to 2060 of SEQ ID NO:119, encodes a 552 amino acid protein (SEQ ID NO:120).

As assed by TaqMan analysis, 33877 mRNA showed restricted tissue expression with the highest normal tissue expression in brain cortex, neutrophils, and kidney. Further TaqMan experiments using an oncology panel demonstrated that 33877 mRNA expression was significantly upregulated in tumors of the breast (2/6), ovary (5/6), and lung (4/5) when compared to their respective normal tissues. On an expanded lung tumor TaqMan panel, 33877 mRNA showed increased expression in 20/35 samples when compared to normal lung tissues.

In situ hybridization experiments conducted on 33877 indicated that 33877 mRNA is expressed at various levels in primary lung (5/6) and ovarian tumors (2/3). Little to no expression was observed in normal tissues surveyed.

Tumor progression is often marked by altered patterns of glycoslylation, which is thought to contribute the invasive and metastatic properties of tumor cells. 33877 is a member of the GalNAC family of glycosyl transferases, which play a critical role in the regulation of cellular glycosylation patterns.

Overexpression of 33877 in tumor cells results in aberrant glycosylation and increased metastatic potential of those cells.

Due to 33877 function and expression, modulators of 33877 activity would be useful in treating human cancers, including but not limited to breast, lung and ovarian cancers. 33877 polypeptides of the present invention are useful in screening for modulators of 33877 activity.

Gene ID 10317

The human 10317 sequence (SEQ ID NO:121), known also as palmitoyl-protein thioesterase precursor (PPT1) or palmitoyl-protein hydrolase, is approximately 2287 nucleotides long including untranslated regions. The coding sequence, located at about nucleic acids 15 to 935 of SEQ ID NO:121, encodes a 306 amino acid protein (SEQ ID NO:122).

As assessed by TaqMan analysis, 10317 mRNA was expressed at higher levels in colon, lung, and breast tumors (single sample pools of 3 tumors) with respect to normal colon, lung, and breast (single sample pools of 3 tissue samples), respectively. 10317 mRNA was also highly expressed in human umbilical vein endothelial cells (HUVEC), brain cortex, hypothalamus and macrophages.

Further TaqMan analysis on an oncology tissue panel indicated that 10317 mRNA was expressed at increased levels in 2/6 breast, in 2/6 lung, and in 1/5 colon tumors when compared to their respective normal tissue samples (3/3 breast, 3/3 lung, 3/3 colon). 10317 mRNA was also expressed at increased levels in proliferating human microvascular endothelial cells (HMVEC) relative to arrested HMVEC.

TaqMan analysis of an angiogenesis panel (fetal vs. adult tissues) indicated that 10317 mRNA was expressed at increased levels in fetal kidney (2/2) relative to adult angiogenic tissue samples (24/24). TaqMan analysis using an additional angiogenesis panel (hemangiomas and necrotic and angiogenic tumors) indicated that 10317 mRNA was expressed at higher levels in 4/8 hemangiomas relative to 1/1 normal skin tissue samples. 10317 mRNA was also expressed at higher levels in necrotic tumors (1/3 breast, 3/3 colon, and 3/3 lung tumors) and in Wilm's tumor relative to normal controls (breast (1/1), colon (1/1), lung (1/1), and kidney (1/1)).

In an expanded lung tumor panel, 10317 mRNA was increased in 14/35 lung tumors when compared to 6/6 normal lung tissue samples.

In a colon ACA tumor panel, 10317 mRNA was increased in 3/15 colon primary tumors when compared to 5/6 normal colon tissue samples and in 3/5 colon tumor metastasis samples when compared to either 5/6 normal colon tissue samples or 6/6 normal liver tissue samples. In a colon tumor metastasis panel, 10317 mRNA was increased in 4/4 colon primary tumors when compared to 3/3 normal colon tissue samples and in 13/16 colon tumor metastasis to the liver when compared to either 3/3 normal colon tissue samples or 3/3 normal liver tissue samples. 10317 mRNA was also increased in 12/14 samples of colon tumor metastasis to other sites when compared to 3/3 normal colon tissue samples.

TaqMan analysis of 10317 mRNA expression in an “endothelial cell in vitro proliferation, arrest and tube formation” panel indicated increased expression in proliferating HUVEC (1/1) and lung HMVEC (3/3) when compared to arrested HUVEC (1/1) and lung HMVEC (3/3). 10317 mRNA expression is also increased in tube formation in HUVEC (5/9) and lung HMVEC (4/6) when compared to arrested HUVEC (1/1) and lung HMVEC (3/3).

In situ experiments conducted on 10317 indicated that 10317 mRNA was expressed in primary lung and breast tumors. Normal breast and lung tissue are essentially negative for this gene. A variety of angiogenic tissues such as fetal kidney, adrenal, and Wilm's tumor are positive for expression.

Didemnin B is a promising natural product whose efficacy may be strongly limited by its widespread toxicities. Aplidine provides evidence that its antitumor activity is not mechanistically linked to its cardiotoxicity. Antisense, knockout, overexpression and synthetic inhibitor studies are consistent with PPT1 being a key target of didemnin B antitumor activity. Inhibition of EF-1a cannot account for its activity. Palmitoylation and depalmitoylation have been demonstrated to affect the subcellular localization and activity of Ras. Inhibition of 10317 will prevent tumors from targeting the activity of important signaling molecules to key subcellular locations, resulting in dysregulation and apoptosis.

Due to 10317 function and expression, modulators of 10317 activity would be useful in treating human cancers, including but not limited to breast, colon and lung cancers, and tumor angiogenesis. 10317 polypeptides of the present invention are useful in screening for modulators of 10317 activity.

Gene ID 10485

The human 10485 sequence (SEQ ID NO:123), known also as selenide water dikinase 1, selenophosphate synthetase 1, or selenium donor protein 1, is approximately 1429 nucleotides long including untranslated regions. The coding sequence, located at about nucleic acids 69 to 1247 of SEQ ID NO:123, encodes a 392 amino acid protein (SEQ ID NO:124).

As assessed by TaqMan analysis, 10485 mRNA is expressed at a 4-fold increased level in a primary colon tumor pool when compared to a normal colon tissue pool. 10485 mRNA is also expressed at a 3-fold increased level in a primary lung tumor pool when compared to a normal lung tissue pool.

In an oncology TaqMan tissue panel, 10485 mRNA expression is increased 2 to 3-fold in primary breast tumor samples, 2 to 7-fold in lung tumor samples and 2 to 3-fold in primary colon tumors when compared to their respective normal samples. 10485 mRNA is expressed at 2-fold increased levels in a pooled colon to liver metastases sample when compared to a normal liver sample.

In the phosphotidylinositol 3-kinase (PI3Kinase) TaqMan panel, 10485 mRNA expression is decreased after 48 hours of treatment with ˜8× the EC 50 of the PI3Kinase in LNCaP (30 uM), MDA-MB-468 (115 uM), MCF Tet Off (30 uM) and NCI-H125 (150 uM) cancer cell lines. 10485 mRNA expression in LNCaP cells decreases 3 fold after 96 hours of treatment. 10485 mRNA expression in MDA-MB-468 cells decreases 3 fold after 48 hours of treatment. 10485 mRNA expression in MCF Tet Off cells decreases 2.6 fold after 48 hours of treatment. 10485 mRNA expression in NCI-H125 cells decreases 6 fold after 96 hours of treatment.

In an expanded lung cancer tissue panel, 10485 mRNA expression is increased 2 to 14-fold in primary lung tumor samples.

In a breast cancer cell model panel, 10485 mRNA shows increased expression in estrogen receptor positive vs. estrogen receptor negative cell lines ranging from 1.2 to 11-fold.

10485, selenophosphate synthetase (human selenium donor protein), is a member of the airs (AIR synthase and relatives) class. This protein encodes an enzyme that synthesizes selenophosphate from selenide and ATP. Selenophosphate is the selenium donor used to synthesize selenocysteine, which is co-translationally incorporated into selenoproteins at in-frame UGA codons. This protein itself contains a selenocysteine residue in its predicted active site. (J. of Health Science; 46(6):399-404 (2000)).

10485 is a human selenium donor protein that is increased in primary lung tumors and is decreased in PI3-kinase inhibited cell lines. Inhibiting 10485 would decrease selenophosphate levels, thereby decreasing the synthesis of an array of selenoproteins. Functionally important selenoproteins are involved in antioxidant defense systems, thyroid metabolism and immune function. Therefore, tumors may have increased requirements for selenoproteins and increased sensitivity to reduced selenoprotein levels.

Due to 10485 function and expression, modulators of 10485 activity would be useful in treating human cancers, including but not limited to breast, colon and lung cancers. 10485 polypeptides of the present invention are useful in screening for modulators of 10485 activity.

Gene ID 25964

The human 25964 sequence (SEQ ID NO:125), known also as 3 beta-hydroxy-delta 5-C27-steroid oxidoreductase, is approximately 1725 nucleotides long including untranslated regions. The coding sequence, located at about nucleic acids 281 to 1390 of SEQ ID NO:125, encodes a 369 amino acid protein (SEQ ID NO:126).

As assessed by TaqMan analysis, 25964 mRNA expression was increased 4.5-fold in pooled prostate tumors relative to pooled normal prostates. 25964 mRNA expression was also elevated 7-fold in pooled colon tumors relative to pooled normal colon. 25964 mRNA was also highly expressed in erythroid cells.

In an oncology TaqMan panel, 25964 mRNA expression was increased in 4/5 breast tumors (3.5 to 17-fold) relative to 3 normal breast samples; in 2/5 ovarian tumors (2 to 4-fold) relative to 3 normal ovary samples; and in 2/6 lung tumors (3.5 to 7-fold) relative to 3 normal lung samples.

In situ hybridization experiments conducted on 25964 confirmed the TaqMan data in that 5/9 ovarian, 3/4 breast, and 2/3 colon carcinomas were moderately positive for expression. Normal tissues, except for liver, are negative for this gene in all cases by both TaqMan and ISH. Normal liver is positive for 25964, however, the colon metastases are slightly more positive.

25964 encodes 3 beta-hydroxy-delta 5-C27-steroid oxidoreductase, which is involved in bile acid biosynthesis. Overexpression of 25964 in tumors may suggest an increased dependence on this gene for cell transformation, proliferation and/or survival. Inhibition of 25964 may therefore result in an inhibition and/or reduction in tumor growth.

Due to 25964 function and expression, modulators of 25964 activity would be useful in treating human cancers, including but not limited to breast, colon, lung and ovarian cancers. 25964 polypeptides of the present invention are useful in screening for modulators of 25964 activity.

Gene ID 14815

The human 14815 sequence (SEQ ID NO:127), an uncharacterized kinase, is approximately 3919 nucleotides long including untranslated regions. The coding sequence, located at about nucleic acids 183 to 3530 of SEQ ID NO:127, encodes a 1115 amino acid protein (SEQ ID NO:128).

As assessed by TaqMan analysis, 14815 mRNA demonstrated very restricted expression in normal tissues with the highest expression found in CNS tissue samples. Additional TaqMan experiments using a panel composed of normal and clinical tumor samples showed that 14815 mRNA was upregulated in breast tumors as compared to normal breast cancer samples. Low expression levels were detected in other tumor types tested.

Expression of 14815 in RNAi transfected cells was <50% of that seen in mock transfected controls.

Treatment of tumor cells with RNAi directed against 14815 results in varying levels of growth inhibition (72 hours post-transfection) in the following cell lines:

TABLE 8

| Cell Line | % Growth Inhibition |
|---|---|
| A549 | 54% (2 of 2 assays) |
| HCT116 | 35% (average, 2 of 3 assays) |
| HT29 | 42% (PLK only 23%) |

Reduction of 14815 expression in tumor cells by transfection with siRNA oligos specific for that gene results in cell growth inhibition. RNAi studies in 3 different cell lines (A549, HT29, and HCT116) showed significant levels of growth inhibition coincident with reduction of 14815 expression. Small molecule inhibitors of 14815 would be expected to have a similar effect on tumor cell growth, and are therefore attractive cancer therapeutic candidates.

Due to 14815 function and expression, modulators of 14815 activity would be useful in treating human cancers, including but not limited to breast cancers. 14815 polypeptides of the present invention are useful in screening for modulators of 14815 activity.

Gene ID 1363

The human 1363 sequence (SEQ ID NO:129), known also as human Beta-adrenergic receptor kinase 1 (ARK1), is approximately 3136 nucleotides long including untranslated regions. The coding sequence, located at about nucleic acids 108 to 2177 of SEQ ID NO:129, encodes a 689 amino acid protein (SEQ ID NO:130).

As assessed by TaqMan analysis, 1363 mRNA demonstrated broad tissue expression with the highest expression levels observed in normal brain cortex, neutrophils, and monocyte progenitors. Additional TaqMan experiments using a panel composed of normal and clinical tumor samples showed that 1363 mRNA expression was upregulated in 2/6 breast tumors, in 2/6 ovarian tumors, in 4/6 lung tumors, and in 1/1 colon-to-liver metastases as compared to their respective normal tissues.

In situ hybridization experiments conducted on 1363 indicated that 1363 is expressed in 3/3 primary colon and in 4/4 lung tumors as well as in 1/2 colon metastasis to the liver. Primary lung tumors are modestly positive compared to colon primary tumors, which highly express 1363. Metastatic colon carcinoma highly expresses 1363 as well. Normal colon and lung epithelium show little to no expression.

Expression of 1363 in RNAi transfected cells was 30% of that seen in mock transfected controls.

Treatment of tumor cells with RNAi directed against 1363 results in varying levels of growth inhibition (72 hours post-transfection) in the following cell lines:

TABLE 9

| Cell Line | % Growth Inhibition |
|---|---|
| A549 | 55% |
| HCT116 | 57% |

RNAi studies in A549 and HCT116 cells showed significant levels of growth inhibition coincident with reduction of 1363 expression. Small molecule inhibitors of 1363 would be expected to have an effect similar to that seen with RNAi on tumor cell growth, and are therefore attractive cancer therapeutic candidates.

Due to 1363 function and expression, modulators of 1363 activity would be useful in treating human cancers, including but not limited to breast, ovarian, colon and lung cancers. 1363 polypeptides of the present invention are useful in screening for modulators of 1363 activity.

Gene ID 1397

The human 1397 sequence (SEQ ID NO:131), known also as human protein kinase CLK2, is approximately 1973 nucleotides long including untranslated regions. The coding sequence, located at about nucleic acids 130 to 1629 of SEQ ID NO:131, encodes a 499 amino acid protein (SEQ ID NO:132).

As assessed by TaqMan analysis, 1397 mRNA demonstrated broad tissue expression with the highest level of expression found in normal brain cortex and erythroid cells. Additional TaqMan experiments using a panel composed of normal and clinical tumor samples showed that 1397 mRNA expression was upregulated in 2/6 breast tumors, in 2/6 ovarian tumors, in 4/6 lung tumors, and in 4/5 colon tumors as compared their respective normal tissues.

In situ hybridization experiments conducted on 1397 indicated that 1397 is expressed in 3/3 primary breast, in 1/2 colon, and in 4/4 lung tumors. Some expression was seen in normal lung and breast tissue, but not to the extent seen in tumor. Normal colon epithelium is negative, but some sticky IFL were seen in the antisense and sense control.

Expression of 1397 in RNAi transfected cells was <60% of that seen in mock transfected controls.

Treatment of tumor cells with RNAi directed against 1397 results in varying levels of growth inhibition (72 hours post-transfection) in the following cell lines:

TABLE 10

| Cell Line | % Growth Inhibition |
|---|---|
| A549 | 57% |
| HCT116 | 44% |

RNAi studies in A549 and HCT116 cells showed significant levels of growth inhibition coincident with reduction of 1397 expression. Small molecule inhibitors of 1397 would be expected to have an effect similar to that seen with RNAi on tumor cell growth, and are therefore attractive cancer therapeutic candidates.

Due to 1397 function and expression, modulators of 1397 activity would be useful in treating human cancers, including but not limited to breast, ovarian, colon and lung cancers. 1397 polypeptides of the present invention are useful in screening for modulators of 1397 activity.

Gene ID 14827

The human 14827 sequence (SEQ ID NO:133), known also as human STE20/SPS1-related proline-alanine rich protein kinase (SPAK), is approximately 3293 nucleotides long including untranslated regions. The coding sequence, located at about nucleic acids 174 to 1817 of SEQ ID NO:133, encodes a 547 amino acid protein (SEQ ID NO:134).

As assessed by TaqMan analysis, 14827 mRNA demonstrated broad tissue expression with the highest level of expression in normal pituitary gland and bladder tissue samples. Additional TaqMan experiments using a panel composed of normal and clinical tumor samples showed that 14827 mRNA expression was upregulated in 4/6 lung tumors and in 4/4 colon tumors as compared to their respective normal tissues.

In situ hybridization experiments conducted on 14827 indicated that 14827 is very positive in 5/5 primary tumors of the lung and in 3/3 colon tumors, as well as in 2/2 colon metastasis to the liver. No expression was seen in normal lung or colon epithelium. Some sticky IFL was seen in the T3 and T7 for normal colon.

Expression of 14827 in RNAi transfected cells was <15% of that seen in mock transfected controls.

Treatment of tumor cells with RNAi directed against 14827 results in varying levels of growth inhibition (72 hours post-transfection) in the following cell lines:

TABLE 11

| Cell Line | % Growth Inhibition |
|---|---|
| A549 | 59% |
| HCT116 | 44% |

RNAi studies in A549 and HCT116 cells showed significant levels of growth inhibition coincident with reduction of 14827 expression. Small molecule inhibitors of 14827 would be expected to have an effect similar to that seen with RNAi on tumor cell growth, and are therefore attractive cancer therapeutic candidates.

Due to 14827 function and expression, modulators of 14827 activity would be useful in treating human cancers, including but not limited to colon and lung cancers. 14827 polypeptides of the present invention are useful in screening for modulators of 14827 activity.

Gene ID 21708

The human 21708 sequence (SEQ ID NO:135), known also as MAST205, is approximately 5737 nucleotides long including untranslated regions. The coding sequence, located at about nucleic acids 284 to 5488 of SEQ ID NO:135, encodes a 1734 amino acid protein (SEQ ID NO:136).

As assessed by TaqMan analysis, 21708 mRNA demonstrated broad tissue expression with the highest level of expression in normal skeletal muscle, heart, and brain cortex. Additional TaqMan experiments using a panel composed of normal and clinical tumor samples showed that 21708 mRNA expression was upregulated in 2/6 breast tumors and in 2/6 lung tumors as compared to their respective normal tissues.

In situ hybridization experiments conducted on 21708 indicated that 21708 is highly expressed in 5/5 primary lung and in 3/3 colon tumors, and in 3/3 colon metastases to the liver. Normal lung was negative. Normal colon epithelium was modestly positive, but less so that primary tumor. Normal lung epithelium was negative.

Expression of 21708 in RNAi transfected cells was <25% of that seen in mock transfected controls.

Treatment of tumor cells with RNAi directed against 21708 results in varying levels of growth inhibition (72 hours post-transfection) in the following cell lines:

TABLE 12

| Cell Line | % Growth Inhibition |
|---|---|
| A549 | 47% |
| HCT116 | 64% |

RNAi studies in A549 and HCT116 cells showed significant levels of growth inhibition coincident with reduction of 21708 expression. Small molecule inhibitors of 21708 would be expected to have an effect similar to that seen with RNAi on tumor cell growth, and are therefore attractive cancer therapeutic candidates.

Due to 21708 function and expression, modulators of 21708 activity would be useful in treating human cancers, including but not limited to breast and lung cancers. 21708 polypeptides of the present invention are useful in screening for modulators of 21708 activity.

Gene ID 3801

The human 3801 sequence (SEQ ID NO:137), which is known as human dual specificity mitogen-activated protein kinase kinase 5 (MPK5), is approximately 2083 nucleotides long including untranslated regions. The partial coding sequence, located at about nucleic acids 297 to 1613 of SEQ ID NO:137, encodes a 438 amino acid protein (SEQ ID NO:138).

As assessed by TaqMan analysis, 3801 mRNA demonstrated broad tissue expression with the highest level of expression in normal skeletal muscle and brain cortex. Additional TaqMan experiments using a panel composed of normal and clinical tumor samples showed that 3801 mRNA expression was upregulated in 6/6 lung tumors and in 2/5 colon tumors as compared to their respective normal tissues.

In situ hybridization experiments conducted on 3801 indicated that 3801 is expressed in 6/6 preliminary lung and in 4/4 colon carcinoma. 3801 mRNA is also expressed very highly in 3/3 colon metastases to the liver. Normal tissues were negative for expression in epithelial cells.

Expression of 3801 in RNAi transfected cells was <30% of that seen in mock transfected controls.

Treatment of tumor cells with RNAi directed against 3801 results in varying levels of growth inhibition (72 hours post-transfection) in the following cell lines:

TABLE 13

| Cell Line | % Growth Inhibition |
|---|---|
| A549 | 43% |
| HCT116 | 58% |

RNAi studies in A549 and HCT116 cells showed significant levels of growth inhibition coincident with reduction of 3801 expression. Small molecule inhibitors of 3801 would be expected to have an effect similar to that seen with RNAi on tumor cell growth, and are therefore attractive cancer therapeutic candidates.

Due to 3801 function and expression, modulators of 3801 activity would be useful in treating human cancers, including but not limited to colon and lung cancers. 3801 polypeptides of the present invention are useful in screening for modulators of 3801 activity.

Gene ID 64698

The human 64698 sequence (SEQ ID NO:139), known also as human sphingosine kinase 2 (SPH2), is approximately 2380 nucleotides long including untranslated regions. The coding sequence, located at about nucleic acids 7 to 1863 of SEQ ID NO:139, encodes a 618 amino acid protein (SEQ ID NO:140).

As assessed by TaqMan analysis, 64698 mRNA demonstrated broad tissue expression with the highest level of expression in normal hypothalamus, brain cortex, and erythroid cells. Additional TaqMan experiments using a panel composed of normal and clinical tumor samples showed that 64698 mRNA expression was upregulated in 2/6 breast tumors, in 6/6 lung tumors and in 1/5 colon tumors as compared to their respective normal tissues.

In situ hybridization experiments conducted on 64698 showed expression of 64698 in 4/4 primary breast tumors, in 5/6 lung tumors, and in 5/5 colon tumors as well as in 3/4 colon metastases to the liver. While expression was variable, most samples showed expression. Some background was seen in infiltrating lymphocytes in lung tissue and in colon tissue. Normal tissues showed some expression, most notably breast and lung tissues. This is consistent with the TaqMan data. Normal colon was negative, but showed sticky IFL in both the sense and antisense slides.

Expression of 64698 in RNAi transfected cells was <35% of that seen in mock transfected controls.

Treatment of tumor cells with RNAi directed against 64698 results in varying levels of growth inhibition (72 hours post-transfection) in the following cell lines:

TABLE 14

| Cell Line | % Growth Inhibition |
|---|---|
| A549 | 53% |
| HCT116 | 49% |

RNAi studies in A549 and HCT116 cells showed significant levels of growth inhibition coincident with reduction of 64698 expression. Small molecule inhibitors of 64698 would be expected to have an effect similar to that seen with RNAi on tumor cell growth, and are therefore attractive cancer therapeutic candidates.

Due to 64698 function and expression, modulators of 64698 activity would be useful in treating human cancers, including but not limited to breast, colon and lung cancers. 64698 polypeptides of the present invention are useful in screening for modulators of 64698 activity.

Gene ID 2179

The human 2179 sequence (SEQ ID NO:141), known also as MOK protein kinase, is approximately 1954 nucleotides long including untranslated regions. The coding sequence, located at about nucleic acids 227 to 1486 of SEQ ID NO:141, encodes a 419 amino acid protein (SEQ ID NO:142).

As assessed by TaqMan analysis using a normal human tissue panel, 2179 mRNA was broadly expressed in normal tissues with the highest expression level observed in normal brain cortex tissue samples. TaqMan experiments performed using a human oncology tissue panel demonstrated that 2179 mRNA expression was upregulated in 2/5 breast tumors, in 1/6 ovarian tumors, in 4/6 lung tumors and in 2/5 colon tumors as compared to their respective normal tissues.

Expression of 2179 in RNAi transfected cells was <50% of that seen in mock transfected controls.

Treatment of tumor cells with RNAi directed against 2179 results in varying levels of growth inhibition (72 hours post-transfection) in the following cell lines:

TABLE 15

| Cell Line | % Growth Inhibition |
|---|---|
| HCT116 | 50% |

Deconvolution experiments in HCT116 cells at 72 h resulted in greater than 55% inhibition of growth in 75% (3/4) of transfected cells.

RNAi studies performed using HCT116 cells showed significant levels of growth inhibition coincident with reduction of 2179 expression. Small molecule inhibitors of 2179 would be attractive cancer therapeutic candidates as they would have an effect similar to that seen with RNAi on tumor cell growth.

Due to 2179 function and expression, modulators of 2179 activity would be useful in treating human cancers, including but not limited to breast, ovarian, colon and lung cancers, and tumor angiogenesis. 2179 polypeptides of the present invention are useful in screening for modulators of 2179 activity.

Gene ID 13249

The human 13249 sequence (SEQ ID NO:143), known also as STE20 like kinase, is approximately 3032 nucleotides long including untranslated regions. The coding sequence, located at about nucleic acids 296 to 2992 of SEQ ID NO:143, encodes a 898 amino acid protein (SEQ ID NO:144).

As assessed by TaqMan analysis using a normal human tissue panel, 13249 mRNA was broadly expressed in normal tissues with the highest expression level observed in normal brain cortex tissue samples. TaqMan experiments performed using a human oncology tissue panel demonstrated that 13249 mRNA expression was upregulated in 1/6 breast tumors, in 1/6 lung tumors and in 2/5 colon tumors as compared to their respective normal tissues.

Expression of 13249 in RNAi transfected cells was <5% of that seen in mock transfected controls.

Treatment of tumor cells with RNAi directed against 13249 results in varying levels of growth inhibition (72 hours post-transfection) in the following cell lines:

TABLE 16

| Cell Line | % Growth Inhibition |
|---|---|
| HCT116 | 51% |

Deconvolution experiments in HCT116 cells at 72 h resulted in greater than 60% inhibition of growth in 100% (4/4) of transfected cells.

RNAi studies in HCT116 cells showed significant levels of growth inhibition coincident with reduction of 13249 expression. Small molecule inhibitors of 13249 would be attractive cancer therapeutic candidates as they would have an effect similar to that seen with RNAi on tumor cell growth.

Due to 13249 function and expression, modulators of 13249 activity would be useful in treating human cancers, including but not limited to breast, ovarian, colon and lung cancers and tumor angiogenesis. 13249 polypeptides of the present invention are useful in screening for modulators of 13249 activity.

Various aspects of the invention are described in further detail in the following subsections:

Screening Assays:

The invention provides a method (also referred to herein as a “screening assay”) for identifying modulators, i.e., candidate or test compounds or agents (e.g., peptides, peptidomimetics, small molecules (organic or inorganic) or other drugs) which bind to 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 proteins, have a stimulatory or inhibitory effect on, for example, 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 expression or 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity, or have a stimulatory or inhibitory effect on, for example, the expression or activity of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 substrate. Compounds identified using the assays described herein may be useful for treating a cancer.

These assays are designed to identify compounds that bind to a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein, bind to other intracellular or extracellular proteins that interact with a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein, and interfere with the interaction of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein with other intercellular or extracellular proteins. For example, in the case of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein, which is a transmembrane receptor-type protein, such techniques can identify ligands for such a receptor. A 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein ligand or substrate can, for example, be used to ameliorate at least one symptom of a cancer. Such compounds may include, but are not limited small molecules, peptides, antibodies, ribozymes, gene therapy vectors and antisense oligonucleotides. Such compounds may also include other cellular proteins.

Compounds identified via assays such as those described herein may be useful, for example, for treating a cancer. In instances whereby a cancer condition results from an overall lower level of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene expression and/or 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein in a cell or tissue, compounds that interact with the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein may include compounds which accentuate or amplify the activity of the bound 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein. Such compounds would bring about an effective increase in the level of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein activity, thus ameliorating symptoms.

In other instances, mutations within the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene may cause aberrant types or excessive amounts of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 proteins to be made which have a deleterious effect that leads to a cancer. Similarly, physiological conditions may cause an excessive increase in 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene expression leading to a cancer. In such cases, compounds that bind to a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein may be identified that inhibit the activity of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein. Assays for testing the effectiveness of compounds identified by techniques such as those described in this section are discussed herein.

In one embodiment, the invention provides assays for screening candidate or test compounds which are substrates of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein or polypeptide or biologically active portion thereof. In another embodiment, the invention provides assays for screening candidate or test compounds which bind to or modulate the activity of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In one embodiment, an assay is a cell-based assay in which a cell which expresses a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein or biologically active portion thereof is contacted with a test compound and the ability of the test compound to modulate 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity is determined. Determining the ability of the test compound to modulate 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity can be accomplished by monitoring, for example, intracellular calcium, IP₃, cAMP, or diacylglycerol concentration, the phosphorylation profile of intracellular proteins, cell proliferation and/or migration, gene expression of, for example, cell surface adhesion molecules or genes associated with cancer, or the activity of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249-regulated transcription factor. The cell can be of mammalian origin, e.g., a cancer cell. In one embodiment, compounds that interact with a receptor domain can be screened for their ability to function as ligands, i.e., to bind to the receptor and modulate a signal transduction pathway. Identification of ligands, and measuring the activity of the ligand-receptor complex, leads to the identification of modulators (e.g., antagonists) of this interaction. Such modulators may be useful in the treatment of a cancer.

The ability of the test compound to modulate 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 binding to a substrate or to bind to 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 can also be determined. Determining the ability of the test compound to modulate 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 binding to a substrate can be accomplished, for example, by coupling the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 substrate with a radioisotope or enzymatic label such that binding of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 substrate to 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 can be determined by detecting the labeled 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 substrate in a complex. 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 could also be coupled with a radioisotope or enzymatic label to monitor the ability of a test compound to modulate 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 binding to a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 substrate in a complex. Determining the ability of the test compound to bind 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 can be accomplished, for example, by coupling the compound with a radioisotope or enzymatic label such that binding of the compound to 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 can be determined by detecting the labeled 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 compound in a complex. For example, compounds (e.g., 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 ligands or substrates) can be labeled with ¹²⁵I, ³⁵S, ¹⁴C, or ³H, either directly or indirectly, and the radioisotope detected by direct counting of radioemmission or by scintillation counting. Compounds can further be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g., a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 ligand or substrate) to interact with 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 without the labeling of any of the interactants. For example, a microphysiometer can be used to detect the interaction of a compound with 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 without the labeling of either the compound or the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 (McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249.

In another embodiment, an assay is a cell-based assay comprising contacting a cell expressing a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 target molecule (e.g., a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 substrate) with a test compound and determining the ability of the test compound to modulate (e.g., stimulate or inhibit) the activity of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 target molecule. Determining the ability of the test compound to modulate the activity of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 target molecule can be accomplished, for example, by determining the ability of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein to bind to or interact with the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 target molecule.

Determining the ability of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein or a biologically active fragment thereof, to bind to or interact with a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 target molecule can be accomplished by one of the methods described above for determining direct binding. In a preferred embodiment, determining the ability of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein to bind to or interact with a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 target molecule can be accomplished by determining the activity of the target molecule. For example, the activity of the target molecule can be determined by detecting induction of a cellular second messenger of the target (i.e., intracellular Ca²⁺, diacylglycerol, IP₃, cAMP), detecting catalytic/enzymatic activity of the target on an appropriate substrate, detecting the induction of a reporter gene (comprising a target-responsive regulatory element operatively linked to a nucleic acid encoding a detectable marker, e.g., luciferase), or detecting a target-regulated cellular response (e.g., gene expression).

In yet another embodiment, an assay of the present invention is a cell-free assay in which a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein or biologically active portion thereof, is contacted with a test compound and the ability of the test compound to bind to the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein or biologically active portion thereof is determined. Preferred biologically active portions of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 proteins to be used in assays of the present invention include fragments which participate in interactions with non-15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 molecules, e.g., fragments with high surface probability scores. Binding of the test compound to the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein can be determined either directly or indirectly as described above. In a preferred embodiment, the assay includes contacting the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein or biologically active portion thereof with a known compound which binds 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 to form an assay mixture, contacting the assay mixture with a test compound, and determining the ability of the test compound to interact with a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein, wherein determining the ability of the test compound to interact with a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein comprises determining the ability of the test compound to preferentially bind to 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 or biologically active portion thereof as compared to the known compound. Compounds that modulate the interaction of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 with a known target protein may be useful in regulating the activity of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein, especially a mutant 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein.

In another embodiment, the assay is a cell-free assay in which a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein or biologically active portion thereof is contacted with a test compound and the ability of the test compound to modulate (e.g., stimulate or inhibit) the activity of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein or biologically active portion thereof is determined. Determining the ability of the test compound to modulate the activity of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein can be accomplished, for example, by determining the ability of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein to bind to a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 target molecule by one of the methods described above for determining direct binding. Determining the ability of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein to bind to a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 target molecule can also be accomplished using a technology such as real-time Biomolecular Interaction Analysis (BIA) (Sjolander, S. and Urbaniczky, C. (1991) Anal. Chem. 63:2338-2345 and Szabo et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In another embodiment, determining the ability of the test compound to modulate the activity of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein can be accomplished by determining the ability of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201;6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein to further modulate the activity of a downstream effector of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 target molecule. For example, the activity of the effector molecule on an appropriate target can be determined or the binding of the effector to an appropriate target can be determined as previously described.

In yet another embodiment, the cell-free assay involves contacting a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein or biologically active portion thereof with a known compound which binds the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein to form an assay mixture, contacting the assay mixture with a test compound, and determining the ability of the test compound to interact with the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein, wherein determining the ability of the test compound to interact with the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein comprises determining the ability of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein to preferentially bind to or modulate the activity of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 target molecule.

In more than one embodiment of the above assay methods of the present invention, it may be desirable to immobilize either 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 or its target molecule to facilitate separation of complexed from uncomplexed forms of one or both of the proteins, as well as to accommodate automation of the assay. Binding of a test compound to a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein, or interaction of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 binding or activity determined using standard techniques.

Other techniques for immobilizing proteins on matrices can also be used in the screening assays of the invention. For example, either a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein or a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 target molecule can be immobilized utilizing conjugation of biotin and streptavidin. Biotinylated 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein or target molecules can be prepared from biotin-NHS(N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein or target molecules but which do not interfere with binding of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein to its target molecule can be derivatized to the wells of the plate, and unbound target or 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein trapped in the wells by antibody conjugation. Methods for detecting such complexes, in addition to those described above for the GST-immobilized complexes, include immunodetection of complexes using antibodies reactive with the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein or target molecule, as well as enzyme-linked assays which rely on detecting an enzymatic activity associated with the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein or target molecule.

In another embodiment, modulators of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 expression are identified in a method wherein a cell is contacted with a candidate compound and the expression of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 mRNA or protein in the cell is determined. The level of expression of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 mRNA or protein in the presence of the candidate compound is compared to the level of expression of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 mRNA or protein in the absence of the candidate compound. The candidate compound can then be identified as a modulator of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 expression based on this comparison. For example, when expression of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 mRNA or protein is greater (statistically significantly greater) in the presence of the candidate compound than in its absence, the candidate compound is identified as a stimulator of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 mRNA or protein expression. Alternatively, when expression of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 mRNA or protein is less (statistically significantly less) in the presence of the candidate compound than in its absence, the candidate compound is identified as an inhibitor of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 mRNA or protein expression. The level of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 mRNA or protein expression in the cells can be determined by methods described herein for detecting 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 mRNA or protein.

In yet another aspect of the invention, the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249, (“15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249-binding proteins” or “15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249-bp”) and are involved in 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity. Such 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249-binding proteins are also likely to be involved in the propagation of signals by the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 proteins or 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 targets as, for example, downstream elements of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249-mediated signaling pathway. Alternatively, such 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249-binding proteins are likely to be 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 inhibitors.

The two-hybrid system is based on the modular nature of most transcription factors, which consist of separable DNA-binding and activation domains. Briefly, the assay utilizes two different DNA constructs. In one construct, the gene that codes for a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein.

In another aspect, the invention pertains to a combination of two or more of the assays described herein. For example, a modulating agent can be identified using a cell-based or a cell free assay, and the ability of the agent to modulate the activity of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein can be confirmed in vivo, e.g., in an animal such as an animal model for a cancer, as described herein.

This invention further pertains to novel agents identified by the above-described screening assays. Accordingly, it is within the scope of this invention to further use an agent identified as described herein in an appropriate animal model. For example, an agent identified as described herein (e.g., a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 modulating agent, an antisense 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 nucleic acid molecule, a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249-specific antibody, or a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

Any of the compounds, including but not limited to compounds such as those identified in the foregoing assay systems, may be tested for the ability to ameliorate at least one symptom of a cancer. Cell-based and animal model-based assays for the identification of compounds exhibiting such an ability to ameliorate at least one symptom of a cancer are described herein.

In addition, animal-based models of a cancer, such as those described herein, may be used to identify compounds capable of treating a cancer. Such animal models may be used as test substrates for the identification of drugs, pharmaceuticals, therapies, and interventions which may be effective in treating a cancer. For example, animal models may be exposed to a compound, suspected of exhibiting an ability to treat a cancer, at a sufficient concentration and for a time sufficient to elicit such an amelioration of at least one symptom of a cancer in the exposed animals. The response of the animals to the exposure may be monitored by assessing the reversal of the symptoms of a cancer before and after treatment.

With regard to intervention, any treatments which reverse any aspect of a cancer (i.e. have an effect on a cancer including but not limited to cancers of the lung, ovary, prostate, breast, colon or other disease state characterized by modulation of angiogenesis) should be considered as candidates for a human cancer therapeutic intervention. Dosages of test agents may be determined by deriving dose-response curves.

Additionally, gene expression patterns may be utilized to assess the ability of a compound to ameliorate at least one symptom of a cancer. For example, the expression pattern of one or more genes may form part of a “gene expression profile” or “transcriptional profile” which may be then be used in such an assessment. “Gene expression profile” or “transcriptional profile”, as used herein, includes the pattern of mRNA expression obtained for a given tissue or cell type under a given set of conditions. Gene expression profiles may be generated, for example, by utilizing a differential display procedure, Northern analysis and/or RT-PCR. In one embodiment, 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene sequences may be used as probes and/or PCR primers for the generation and corroboration of such gene expression profiles.

Gene expression profiles may be characterized for known states, either cancer or normal, within the cell- and/or animal-based model systems. Subsequently, these known gene expression profiles may be compared to ascertain the effect a test compound has to modify such gene expression profiles, and to cause the profile to more closely resemble that of a more desirable profile.

For example, administration of a compound may cause the gene expression profile of a cancer disease model system to more closely resemble the control system. Administration of a compound may, alternatively, cause the gene expression profile of a control system to begin to mimic a cancer or a cancer disease state. Such a compound may, for example, be used in further characterizing the compound of interest, or may be used in the generation of additional animal models.

Cell- and Animal-Based Model Systems

Described herein are cell- and animal-based systems which act as models for cancer. These systems may be used in a variety of applications. For example, the cell- and animal-based model systems may be used to further characterize differentially expressed genes associated with a cancer, e.g., 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249. In addition, animal- and cell-based assays may be used as part of screening strategies designed to identify compounds which are capable of ameliorating at least one symptom of a cancer, as described, below. Thus, the animal- and cell-based models may be used to identify drugs, pharmaceuticals, therapies and interventions which may be effective in treating a cancer. Furthermore, such animal models may be used to determine the LD50 and the ED50 in animal subjects, and such data can be used to determine the in vivo efficacy of potential cancer treatments.

Animal-Based Systems

Animal-based model systems of cancer may include, but are not limited to, non-recombinant and engineered transgenic animals.

Non-recombinant animal models for cancer may include, for example, genetic models.

Models for studying angiogenesis in vivo include tumor cell-induced angiogenesis and tumor metastasis (Hoffman, R M (1998-99) Cancer Metastasis Rev. 17:271-277; Holash, J et al. (1999) Oncogene 18:5356-5362; Li, C Y et al. (2000) J. Natl Cancer Inst. 92:143-147), matrix induced angiogenesis (U.S. Pat. No. 5,382,514), the disc angiogenesis system (Kowalski, J. et al. (1992) Exp. Mol. Pathol. 56:1-19), the rodent mesenteric-window angiogenesis assay (Norrby, K (1992) EXS 61:282-286), experimental choroidal neovascularization in the rat (Shen, W Y et al. (1998) Br. J. Ophthalmol. 82:1063-1071), and the chick embryo development (Brooks, P C et al. Methods Mol. Biol. (1999) 129:257-269) and chick embryo chorioallantoic membrane (CAM) models (McNatt L G et al. (1999) J. Ocul. Pharmacol. Ther. 15:413-423; Ribatti, D et al. (1996) Int. J. Dev. Biol. 40:1189-1197), and are reviewed in Ribatti, D and Vacca, A (1999) Int. J. Biol. Markers 14:207-213.

Animal based models for studying tumorigenesis in vivo are well known in the art (reviewed in Animal Models of Cancer Predisposition Syndromes, Hiai, H and Hino, O (eds.) 1999, Progress in Experimental Tumor Research, Vol. 35; Clarke A R Carcinogenesis (2000) 21:43541) and include, for example, carcinogen-induced tumors (Rithidech, K et al. Mutat Res (1999) 428:33-39; Miller, M L et al. Environ Mol Mutagen (2000) 35:319-327), injection and/or transplantation of tumor cells into an animal, as well as animals bearing mutations in growth regulatory genes, for example, oncogenes (e.g., ras) (Arbeit, J M et al. Am J Pathol (1993) 142:1187-1197; Sinn, E et al. Cell (1987) 49:465-475; Thorgeirsson, S S et al. Toxicol Lett (2000) 112-113:553-555) and tumor suppressor genes (e.g., p53) (Vooijs, M et al. Oncogene (1999) 18:5293-5303; Clark A R Cancer Metast Rev (1995) 14:125-148; Kumar, T R et al. J Intern Med (1995) 238:233-238; Donehower, L A et al. (1992) Nature 356215-221). Furthermore, experimental model systems are available for the study of, for example, ovarian cancer (Hamilton, T C et al. Semin Oncol (1984) 11:285-298; Rahman, N A et al. Mol Cell Endocrinol (1998) 145:167-174; Beamer, W G et al. Toxicol Pathol (1998) 26:704-710), gastric cancer (Thompson, J et al. Int J Cancer (2000) 86:863-869; Fodde, R et al. Cytogenet Cell Genet (1999) 86:105-111), breast cancer (Li, M et al. Oncogene (2000) 19:1010-1019; Green, J E et al. Oncogene (2000) 19:1020-1027), melanoma (Satyamoorthy, K et al. Cancer Metast Rev (1999) 18:401-405), and prostate cancer (Shirai, T et al. Mutat Res (2000) 462:219-226; Bostwick, D G et al. Prostate (2000) 43:286-294).

Additionally, animal models exhibiting a cancer may be engineered by using, for example, 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene sequences described above, in conjunction with techniques for producing transgenic animals that are well known to those of skill in the art. For example, 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene sequences may be introduced into, and overexpressed in, the genome of the animal of interest, or, if endogenous 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene sequences are present, they may either be overexpressed or, alternatively, be disrupted in order to underexpress or inactivate 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene expression.

The host cells of the invention can also be used to produce non-human transgenic animals. For example, in one embodiment, a host cell of the invention is a fertilized oocyte or an embryonic stem cell into which 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249-coding sequences have been introduced. Such host cells can then be used to create non-human transgenic animals in which exogenous 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 sequences have been introduced into their genome or homologous recombinant animals in which endogenous 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 sequences have been altered. Such animals are useful for studying the function and/or activity of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 and for identifying and/or evaluating modulators of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene has been altered by homologous recombination between the endogenous gene and an exogenous DNA molecule introduced into a cell of the animal, e.g., an embryonic cell of the animal, prior to development of the animal.

A transgenic animal used in the methods of the invention can be created by introducing a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249-encoding nucleic acid into the male pronuclei of a fertilized oocyte, e.g., by microinjection, retroviral infection, and allowing the oocyte to develop in a pseudopregnant female foster animal. The 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 cDNA sequence can be introduced as a transgene into the genome of a non-human animal. Alternatively, a nonhuman homologue of a human 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene, such as a mouse or rat 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene, can be used as a transgene. Alternatively, a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene homologue, such as another 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 family member, can be isolated based on hybridization to the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 cDNA sequences and used as a transgene. Intronic sequences and polyadenylation signals can also be included in the transgene to increase the efficiency of expression of the transgene. A tissue-specific regulatory sequence(s) can be operably linked to a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 transgene to direct expression of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein to particular cells. Methods for generating transgenic animals via embryo manipulation and microinjection, particularly animals such as mice, have become conventional in the art and are described, for example, in U.S. Pat. Nos. 4,736,866 and 4, 870, 009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 transgene in its genome and/or expression of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 mRNA in tissues or cells of the animals. A transgenic founder animal can then be used to breed additional animals carrying the transgene. Moreover, transgenic animals carrying a transgene encoding a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein can further be bred to other transgenic animals carrying other transgenes.

To create a homologous recombinant animal, a vector is prepared which contains at least a portion of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene into which a deletion, addition or substitution has been introduced to thereby alter, e.g., functionally disrupt, the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene. The 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene can be a human gene but more preferably, is a non-human homologue of a human 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene. For example, a rat 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene can be used to construct a homologous recombination nucleic acid molecule, e.g., a vector, suitable for altering an endogenous 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene in the mouse genome. In a preferred embodiment, the homologous recombination nucleic acid molecule is designed such that, upon homologous recombination, the endogenous 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene is functionally disrupted (i.e., no longer encodes a functional protein; also referred to as a “knock out” vector). Alternatively, the homologous recombination nucleic acid molecule can be designed such that, upon homologous recombination, the endogenous 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene is mutated or otherwise altered but still encodes functional protein (e.g., the upstream regulatory region can be altered to thereby alter the expression of the endogenous 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein). In the homologous recombination nucleic acid molecule, the altered portion of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene is flanked at its 5′ and 3′ ends by additional nucleic acid sequence of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene to allow for homologous recombination to occur between the exogenous 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene carried by the homologous recombination nucleic acid molecule and an endogenous 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene in a cell, e.g., an embryonic stem cell. The additional flanking 15986, 2188; 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 nucleic acid sequence is of sufficient length for successful homologous recombination with the endogenous gene. Typically, several kilobases of flanking DNA (both at the 5′ and 3′ ends) are included in the homologous recombination nucleic acid molecule (see, e.g., Thomas, K. R. and Capecchi, M. R. (1987) Cell 51:503 for a description of homologous recombination vectors). The homologous recombination nucleic acid molecule is introduced into a cell, e.g., an embryonic stem cell line (e.g., by electroporation) and cells in which the introduced 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene has homologously recombined with the endogenous 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene are selected (see e.g., Li, E. et al. (1992) Cell 69:915). The selected cells can then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination nucleic acid molecules, e.g., vectors, or homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-human animals for use in the methods of the invention can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter Gₒ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

The 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 transgenic animals that express 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 mRNA or a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 peptide (detected immunocytochemically, using antibodies directed against 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 epitopes) at easily detectable levels should then be further evaluated to identify those animals which display a characteristic cancer.

Cell-Based Systems

Cells that contain and express 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene sequences which encode a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein, and, further, exhibit cellular phenotypes associated with a cancer, may be used to identify compounds that exhibit an effect on a cancer. Such cells may include non-recombinant monocyte cell lines, such as U937 (ATCC# CRL-1593), THP-1 (ATCC#TIB-202), and P388D1 (ATCC# TIB-63); endothelial cells such as human umbilical vein endothelial cells (HUVECs), human microvascular endothelial cells (HMVEC), and bovine aortic endothelial cells (BAECs); as well as generic mammalian cell lines such as HeLa cells and COS cells, e.g., COS-7 (ATCC# CRL-1651), lung, colon, breast, prostate or ovarian cancer cell lines. Further, such cells may include recombinant, transgenic cell lines. For example, the cancer animal models of the invention, discussed above, may be used to generate cell lines, containing one or more cell types involved in cancer, that can be used as cell culture models for this disorder. While primary cultures derived from the cancer model transgenic animals of the invention may be utilized, the generation of continuous cell lines is preferred. For examples of techniques which may be used to derive a continuous cell line from the transgenic animals, see Small et al., (1985) Mol. Cell Biol. 5:642-648.

Alternatively, cells of a cell type known to be involved in cancer may be transfected with sequences capable of increasing or decreasing the amount of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene expression within the cell. For example, 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene sequences may be introduced into, and overexpressed in, the genome of the cell of interest, or, if endogenous 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene sequences are present, they may be either overexpressed or, alternatively disrupted in order to underexpress or inactivate 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene expression.

In order to overexpress a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene, the coding portion of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene may be ligated to a regulatory sequence which is capable of driving gene expression in the cell type of interest, e.g., an endothelial cell. Such regulatory regions will be well known to those of skill in the art, and may be utilized in the absence of undue experimentation. Recombinant methods for expressing target genes are described above.

For underexpression of an endogenous 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene sequence, such a sequence may be isolated and engineered such that when reintroduced into the genome of the cell type of interest, the endogenous 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 alleles will be inactivated. Preferably, the engineered 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 sequence is introduced via gene targeting such that the endogenous 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 sequence is disrupted upon integration of the engineered 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 sequence into the cell's genome. Transfection of host cells with 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 genes is discussed, above.

Cells treated with compounds or transfected with 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 genes can be examined for phenotypes associated with cancer.

Transfection of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 nucleic acid may be accomplished by using standard techniques (described in, for example, Ausubel (1989) supra). Transfected cells should be evaluated for the presence of the recombinant 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene sequences, for expression and accumulation of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 mRNA, and for the presence of recombinant 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein production. In instances wherein a decrease in 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene expression is desired, standard techniques may be used to demonstrate whether a decrease in endogenous 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene expression and/or in 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein production is achieved.

Cellular models for the study of angiogenesis include models of endothelial cell differentiation on Matrigel (Baatout, S. et al. (1996) Rom. J. Intern. Med. 34:263-269; Benelli, R et al. (1999) Int. J. Biol. Markers 14:243-246), embryonic stem cell models of vascular morphogenesis (Doetschman, T. et al. (1993) Hypertension 22:618-629), the culture of microvessel fragments in physiological gels (Hoying, J B et al. (1996) In Vitro Cell Dev. Biol. Anim. 32: 409-419; U.S. Pat. No. 5,976,782), and the treatment of endothelial cells and smooth muscle cells with atherogenic and angiogenic factors including growth factors and cytokines (e.g., IL-1β, PDGF, TNFα, VEGF), homocysteine, and LDL. In vitro angiogenesis models are described in, for example, Black, A F et al. (1999) Cell Biol. Toxicol. 15:81-90.

Cellular models for the study of tumorigenesis are known in the art, and include cell lines derived from clinical tumors, cells exposed to chemotherapeutic agents, cells exposed to carcinogenic agents, and cell lines with genetic alterations in growth regulatory genes, for example, oncogenes (e.g., ras) and tumor suppressor genes (e.g., p53).

Also provided are cells or a purified preparation thereof, e.g., human cells, in which an endogenous 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 is under the control of a regulatory sequence that does not normally control the expression of the endogenous 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene. The expression characteristics of an endogenous gene within a cell, e.g., a cell line or microorganism, can be modified by inserting a heterologous DNA regulatory element into the genome of the cell such that the inserted regulatory element is operably linked to the endogenous 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene. For example, an endogenous 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene, e.g., a gene which is “transcriptionally silent,” e.g., not normally expressed, or expressed only at very low levels, may be activated by inserting a regulatory element which is capable of promoting the expression of a normally expressed gene product in that cell. Techniques such as targeted homologous recombinations, can be used to insert the heterologous DNA as described in, e.g., Chappel, U.S. Pat. No. 5,272,071; WO 91/06667, published on May 16, 1991.

Predictive Medicine:

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein and/or nucleic acid expression as well as 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity, in the context of a biological sample (e.g., blood, serum, cells, e.g., endothelial cells, or tissue, e.g., vascular tissue, bladder tissue or prostate tissue) to thereby determine whether an individual is afflicted with a predisposition or is experiencing a cancer. The invention also provides for prognostic (or predictive) assays for determining whether an individual is at risk of developing a cancer. For example, mutations in a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene can be assayed for in a biological sample. Such assays can be used for prognostic or predictive purpose to thereby phophylactically treat an individual prior to the onset of a cancer.

Another aspect of the invention pertains to monitoring the influence of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 modulators (e.g., anti-15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 antibodies or 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 ribozymes) on the expression or activity of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 in clinical trials.

These and other agents are described in further detail in the following sections.

Diagnostic Assays

To determine whether a subject is afflicted with a disease, a biological sample may be obtained from a subject and the biological sample may be contacted with a compound or an agent capable of detecting a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein or nucleic acid (e.g., mRNA or genomic DNA) that encodes a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein, in the biological sample. A preferred agent for detecting 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 mRNA or genomic DNA is a labeled nucleic acid probe capable of hybridizing to 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 mRNA or genomic DNA. The nucleic acid probe can be, for example, the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 nucleic acid set forth in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141 or 143 or a portion thereof, such as an oligonucleotide of at least 15, 20, 25, 30, 25, 40, 45, 50, 100, 250 or 500 nucleotides in length and sufficient to specifically hybridize under stringent conditions to 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 mRNA or genomic DNA. Other suitable probes for use in the diagnostic assays of the invention are described herein.

A preferred agent for detecting 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein in a sample is an antibody capable of binding to 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

The term “biological sample” is intended to include tissues, cells, and biological fluids isolated from a subject, as well as tissues, cells, and fluids present within a subject. That is, the detection method of the invention can be used to detect 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 mRNA, protein, or genomic DNA in a biological sample in vitro as well as in vivo. For example, in vitro techniques for detection of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 mRNA include Northern hybridizations and in situ hybridizations. In vitro techniques for detection of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein include enzyme linked immunosorbent assays (ELISAs), Western blots, immunoprecipitations and immunofluorescence. In vitro techniques for detection of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 genomic DNA include Southern hybridizations. Furthermore, in vivo techniques for detection of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein include introducing into a subject a labeled anti-15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 antibody. For example, the antibody can be labeled with a radioactive marker whose presence and location in a subject can be detected by standard imaging techniques.

In another embodiment, the methods further involve obtaining a control biological sample from a control subject, contacting the control sample with a compound or agent capable of detecting 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein, mRNA, or genomic DNA, such that the presence of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein, mRNA or genomic DNA is detected in the biological sample, and comparing the presence of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein, mRNA or genomic DNA in the control sample with the presence of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein, mRNA or genomic DNA in the test sample.

Prognostic Assays

The present invention further pertains to methods for identifying subjects having or at risk of developing a disease associated with aberrant 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 expression or activity.

As used herein, the term “aberrant” includes a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 expression or activity which deviates from the wild type 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 expression or activity. Aberrant expression or activity includes increased or decreased expression or activity, as well as expression or activity which does not follow the wild type developmental pattern of expression or the subcellular pattern of expression. For example, aberrant 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 expression or activity is intended to include the cases in which a mutation in the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene causes the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene to be under-expressed or over-expressed and situations in which such mutations result in a non-functional 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein or a protein which does not function in a wild-type fashion, e.g., a protein which does not interact with a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 substrate, or one which interacts with a non-15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 substrate.

The assays described herein, such as the preceding diagnostic assays or the following assays, can be used to identify a subject having or at risk of developing a disease. A biological sample may be obtained from a subject and tested for the presence or absence of a genetic alteration. For example, such genetic alterations can be detected by ascertaining the existence of at least one of 1) a deletion of one or more nucleotides from a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene, 2) an addition of one or more nucleotides to a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene, 3) a substitution of one or more nucleotides of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene, 4) a chromosomal rearrangement of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene, 5) an alteration in the level of a messenger RNA transcript of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene, 6) aberrant modification of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene, such as of the methylation pattern of the genomic DNA, 7) the presence of a non-wild type splicing pattern of a messenger RNA transcript of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene, 8) a non-wild type level of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249-protein, 9) allelic loss of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene, and 10) inappropriate post-translational modification of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249-protein.

As described herein, there are a large number of assays known in the art which can be used for detecting genetic alterations in a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene. For example, a genetic alteration in a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene may be detected using a probe/primer in a polymerase chain reaction (PCR) (see, e.g., U.S. Pat. Nos. 4,683,195 and 4, 683, 202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene (see Abravaya et al. (1995) Nucleic Acids Res. 23:675-682). This method includes collecting a biological sample from a subject, isolating nucleic acid (e.g., genomic DNA, mRNA or both) from the sample, contacting the nucleic acid sample with one or more primers which specifically hybridize to a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene under conditions such that hybridization and amplification of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al. (1990) Proc. Natl. Acad. Sci. USA 87:1874-1878), transcriptional amplification system (Kwoh, D. Y. et al. (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene from a biological sample can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 can be identified by hybridizing biological sample derived and control nucleic acids, e.g., DNA or RNA, to high density arrays containing hundreds or thousands of oligonucleotide probes (Cronin, M. T. et al. (1996) Human Mutation 7:244-255; Kozal, M. J. et al. (1996) Nature Medicine 2:753-759). For example, genetic mutations in 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 can be identified in two dimensional arrays containing light-generated DNA probes as described in Cronin, M. T. et al. (1996) supra. Briefly, a first hybridization array of probes can be used to scan through long stretches of DNA in a sample and control to identify base changes between the sequences by making linear arrays of sequential, overlapping probes. This step allows for the identification of point mutations. This step is followed by a second hybridization array that allows for the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene in a biological sample and detect mutations by comparing the sequence of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 in the biological sample with the corresponding wild-type (control) sequence. Examples of sequencing reactions include those based on techniques developed by Maxam and Gilbert (1977) Proc. Natl. Acad. Sci. USA 74:560) or Sanger (1977) Proc. Natl. Acad. Sci. USA 74:5463). It is also contemplated that any of a variety of automated sequencing procedures can be utilized when performing the diagnostic assays (Naeve, C. W. (1995) Biotechniques 19:448-53), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad Sci USA 85:4397 and Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 sequence, e.g., a wild-type 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 genes. For example, single strand conformation polymorphism (SSCP) may be used to detect differences in electrophoretic mobility between mutant and wild type nucleic acids (Orita et al. (1989) Proc Natl. Acad. Sci USA: 86:2766; see also Cotton (1993) Mutat. Res. 285:125-144 and Hayashi (1992) Genet. Anal. Tech. Appl. 9:73-79). Single-stranded DNA fragments of sample and control 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to ensure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

Furthermore, the prognostic assays described herein can be used to determine whether a subject can be administered a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 modulator (e.g., an agonist, antagonist, peptidomimetic, protein, peptide, nucleic acid, or small molecule) to effectively treat a disease.

Monitoring of Effects During Clinical Trials

The present invention further provides methods for determining the effectiveness of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 modulator (e.g., a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 modulator identified herein) in treating a disease. For example, the effectiveness of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 modulator in increasing 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene expression, protein levels, or in upregulating 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity, can be monitored in clinical trials of subjects exhibiting decreased 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene expression, protein levels, or downregulated 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity. Alternatively, the effectiveness of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 modulator in decreasing 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene expression, protein levels, or in downregulating 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity, can be monitored in clinical trials of subjects exhibiting increased 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene expression, protein levels, or 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity. In such clinical trials, the expression or activity of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene, and preferably, other genes that have been implicated in nociception can be used as a “read out” or marker of the phenotype of a particular cell.

For example, and not by way of limitation, genes, including 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249, that are modulated in cells by treatment with an agent which modulates 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity (e.g., identified in a screening assay as described herein) can be identified. Thus, to study the effect of agents which modulate 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity on subjects suffering from a cancer in, for example, a clinical trial, cells can be isolated and RNA prepared and analyzed for the levels of expression of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 and other genes implicated in the cancer. The levels of gene expression (e.g., a gene expression pattern) can be quantified by Northern blot analysis or RT-PCR, as described herein, or alternatively by measuring the amount of protein produced, by one of the methods described herein, or by measuring the levels of activity of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 or other genes. In this way, the gene expression pattern can serve as a marker, indicative of the physiological response of the cells to the agent which modulates 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity. This response state may be determined before, and at various points during treatment of the individual with the agent which modulates 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity.

In a preferred embodiment, the present invention provides a method for monitoring the effectiveness of treatment of a subject with an agent which modulates 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity (e.g., an agonist, antagonist, peptidomimetic, protein, peptide, nucleic acid, or small molecule identified by the screening assays described herein) including the steps of (i) obtaining a pre-administration sample from a subject prior to administration of the agent; (ii) detecting the level of expression of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein, mRNA, or genomic DNA in the pre-administration sample; (iii) obtaining one or more post-administration samples from the subject; (iv) detecting the level of expression or activity of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein, mRNA, or genomic DNA in the post-administration samples; (v) comparing the level of expression or activity of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein, mRNA, or genomic DNA in the pre-administration sample with the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein, mRNA, or genomic DNA in the post administration sample or samples; and (vi) altering the administration of the agent to the subject accordingly. For example, increased administration of the agent may be desirable to increase the expression or activity of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 to higher levels than detected, i.e., to increase the effectiveness of the agent. Alternatively, decreased administration of the agent may be desirable to decrease expression or activity of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 to lower levels than detected, i.e. to decrease the effectiveness of the agent. According to such an embodiment, 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 expression or activity may be used as an indicator of the effectiveness of an agent, even in the absence of an observable phenotypic response.

Methods of Treatment:

The present invention provides for both prophylactic and therapeutic methods of treating a subject, e.g., a human, at risk of (or susceptible to) a disease. With regard to both prophylactic and therapeutic methods of treatment, such treatments may be specifically tailored or modified, based on knowledge obtained from the field of pharmacogenomics. “Pharmacogenomics,” as used herein, refers to the application of genomics technologies such as gene sequencing, statistical genetics, and gene expression analysis to drugs in clinical development and on the market. More specifically, the term refers to the study of how a patient's genes determine his or her response to a drug (e.g., a patient's “drug response phenotype”, or “drug response genotype”).

Thus, another aspect of the invention provides methods for tailoring an subject's prophylactic or therapeutic treatment with either the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 molecules of the present invention or 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

Prophylactic Methods

In one aspect, the invention provides a method for preventing in a subject, a disease by administering to the subject an agent which modulates 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 expression or 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity. Subjects at risk for a cancer, e.g., lung, colon, prostate, ovarian or breast cancer, can be identified by, for example, any or a combination of the diagnostic or prognostic assays described herein. Administration of a prophylactic agent can occur prior to the manifestation of symptoms characteristic of aberrant 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 expression or activity, such that a disease is prevented or, alternatively, delayed in its progression. Depending on the type of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 aberrancy, for example, a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249, 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 agonist or 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 antagonist agent can be used for treating the subject. The appropriate agent can be determined based on screening assays described herein.

Therapeutic Methods

Described herein are methods and compositions whereby a cancer may be ameliorated. Certain cancers are brought about, at least in part, by an excessive level of a gene product, or by the presence of a gene product exhibiting an abnormal or excessive activity. As such, the reduction in the level and/or activity of such gene products would bring about the amelioration of at least one symptom of a cancer. Techniques for the reduction of gene expression levels or the activity of a protein are discussed below.

Alternatively, certain other cancers are brought about, at least in part, by the absence or reduction of the level of gene expression, or a reduction in the level of a protein's activity. As such, an increase in the level of gene expression and/or the activity of such proteins would bring about the amelioration of at least one symptom of a cancer.

In some cases, the up-regulation of a gene in a disease state reflects a protective role for that gene product in responding to the disease condition. Enhancement of such a gene's expression, or the activity of the gene product, will reinforce the protective effect it exerts. Some cancerous disease states may result from an abnormally low level of activity of such a protective gene. In these cases also, an increase in the level of gene expression and/or the activity of such gene products would bring about the amelioration of a least one symptom of a cancer. Techniques for increasing target gene expression levels or target gene product activity levels are discussed herein.

Accordingly, another aspect of the invention pertains to methods of modulating 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 expression or activity for therapeutic purposes. Accordingly, in an exemplary embodiment, the modulatory method of the invention involves contacting a cell with a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 or agent that modulates one or more of the activities of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein activity associated with the cell (e.g., an endothelial cell, ovarian cell, bladder cell and prostate cell). An agent that modulates 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein activity can be an agent as described herein, such as a nucleic acid or a protein, a naturally-occurring target molecule of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein (e.g., a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 ligand or substrate), a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 antibody, a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 agonist or antagonist, a peptidomimetic of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 agonist or antagonist, or other small molecule. In one embodiment, the agent stimulates one or more 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activities. Examples of such stimulatory agents include active 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein and a nucleic acid molecule encoding 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 that has been introduced into the cell. In another embodiment, the agent inhibits one or more 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activities. Examples of such inhibitory agents include antisense 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 nucleic acid molecules, anti-15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 antibodies, and 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein or nucleic acid molecule. In one embodiment, the method involves administering an agent (e.g., an agent identified by a screening assay described herein), or combination of agents that modulates (e.g., upregulates or downregulates) 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 expression or activity. In another embodiment, the method involves administering a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein or nucleic acid molecule as therapy to compensate for reduced, aberrant, or unwanted 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 expression or activity.

Stimulation of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity is desirable in situations in which 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 is abnormally downregulated and/or in which increased 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877; 10317, 10485, 25964, 14815, 1363, 1397, 14827, 21708, 3801, 64698, 2179 or 13249 activity is likely to have a beneficial effect. Likewise, inhibition of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity is desirable in situations in which 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 is abnormally upregulated and/or in which decreased 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity is likely to have a beneficial effect.

Methods for Inhibiting Target Gene Expression, Synthesis, or Activity

As discussed above, genes involved in cancer may cause such disorders via an increased level of gene activity. In some cases, such up-regulation may have a causative or exacerbating effect on the disease state. A variety of techniques may be used to inhibit the expression, synthesis, or activity of such genes and/or proteins.

For example, compounds such as those identified through assays described above, which exhibit inhibitory activity, may be used in accordance with the invention to ameliorate at least one symptom of a cancer. Such molecules may include, but are not limited to, small organic molecules, peptides, antibodies, and the like.

For example, compounds can be administered that compete with endogenous ligand for the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein. The resulting reduction in the amount of ligand-bound 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein will modulate endothelial cell physiology. Compounds that can be particularly useful for this purpose include, for example, soluble proteins or peptides, such as peptides comprising one or more of the extracellular domains, or portions and/or analogs thereof, of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein, including, for example, soluble fusion proteins such as Ig-tailed fusion proteins. (For a discussion of the production of Ig-tailed fusion proteins, see, for example, U.S. Pat. No. 5,116,964). Alternatively, compounds, such as ligand analogs or antibodies, that bind to the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 receptor site, but do not activate the protein, (e.g., receptor-ligand antagonists) can be effective in inhibiting 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein activity.

Further, antisense and ribozyme molecules which inhibit expression of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene may also be used in accordance with the invention to inhibit aberrant 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene activity. Still further, triple helix molecules may be utilized in inhibiting aberrant 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene activity.

The antisense nucleic acid molecules used in the methods of the invention are typically administered to a subject or generated in situ such that they hybridize with or bind to cellular mRNA and/or genomic DNA encoding a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, an antisense nucleic acid molecule used in the methods of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid used in the methods of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 mRNA transcripts to thereby inhibit translation of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 mRNA. A ribozyme having specificity for a 32457-encoding nucleic acid can be designed based upon the nucleotide sequence of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 cDNA disclosed herein (i.e., SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141 or 143). For example, a derivative of a Tetrahymena L-19 IVS RNA can be constructed in which the nucleotide sequence of the active site is complementary to the nucleotide sequence to be cleaved in a, 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249-encoding mRNA (see, for example, Cech et al. U.S. Pat. No. 4,987,071; and Cech et al. U.S. Pat. No. 5,116,742). Alternatively, 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 mRNA can be used to select a catalytic RNA having a specific ribonuclease activity from a pool of RNA molecules (see, for example, Bartel, D. and Szostak, J. W. (1993) Science 261:1411-1418).

15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene expression can also be inhibited by targeting nucleotide sequences complementary to the regulatory region of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 (e.g., the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 promoter and/or enhancers) to form triple helical structures that prevent transcription of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene in target cells (see, for example, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15).

Antibodies that are both specific for the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein and interfere with its activity may also be used to modulate or inhibit 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein function. Such antibodies may be generated using standard techniques described herein, against the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein itself or against peptides corresponding to portions of the protein. Such antibodies include but are not limited to polyclonal, monoclonal, Fab fragments, single chain antibodies, or chimeric antibodies.

In instances where the target gene protein is intracellular and whole antibodies are used, internalizing antibodies may be preferred. Lipofectin liposomes may be used to deliver the antibody or a fragment of the Fab region which binds to the target epitope into cells. Where fragments of the antibody are used, the smallest inhibitory fragment which binds to the target protein's binding domain is preferred. For example, peptides having an amino acid sequence corresponding to the domain of the variable region of the antibody that binds to the target gene protein may be used. Such peptides may be synthesized chemically or produced via recombinant DNA technology using methods well known in the art (described in, for example, Creighton (1983), supra; and Sambrook et al. (1989) supra). Single chain neutralizing antibodies which bind to intracellular target gene epitopes may also be administered. Such single chain antibodies may be administered, for example, by expressing nucleotide sequences encoding single-chain antibodies within the target cell population by utilizing, for example, techniques such as those described in Marasco et al. (1993) Proc. Natl. Acad. Sci. USA 90:7889-7893).

In some instances, the target gene protein is extracellular, or is a transmembrane protein, such as the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein. Antibodies that are specific for one or more extracellular domains of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein, for example, and that interfere with its activity, are particularly useful in treating cancer or a cancer. Such antibodies are especially efficient because they can access the target domains directly from the bloodstream. Any of the administration techniques described below which are appropriate for peptide administration may be utilized to effectively administer inhibitory target gene antibodies to their site of action.

Methods for Restoring or Enhancing Target Gene Activity

Genes that cause a cancer may be underexpressed within the cancer. Alternatively, the activity of the protein products of such genes may be decreased, leading to the development of cancer. Such down-regulation of gene expression or decrease of protein activity might have a causative or exacerbating effect on the disease state.

In some cases, genes that are up-regulated in the disease state might be exerting a protective effect. A variety of techniques may be used to increase the expression, synthesis, or activity of genes and/or proteins that exert a protective effect in response to a cancer.

Described in this section are methods whereby the level of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity may be increased to levels wherein the symptoms of the cancer are ameliorated. The level of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity may be increased, for example, by either increasing the level of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene expression or by increasing the level of active 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein which is present.

For example, a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein, at a level sufficient to ameliorate at least one symptom of a cancer may be administered to a patient exhibiting such symptoms. Any of the techniques discussed below may be used for such administration. One of skill in the art will readily know how to determine the concentration of effective, non-toxic doses of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein, utilizing techniques such as those described below.

Additionally, RNA sequences encoding a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein may be directly administered to a patient exhibiting a cancer, at a concentration sufficient to produce a level of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein such that a cancer are ameliorated. Any of the techniques discussed below, which achieve intracellular administration of compounds, such as, for example, liposome administration, may be used for the administration of such RNA molecules. The RNA molecules may be produced, for example, by recombinant techniques such as those described herein.

Further, subjects may be treated by gene replacement therapy. One or more copies of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene, or a portion thereof, that directs the production of a normal 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein with 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 function, may be inserted into cells using vectors which include, but are not limited to adenovirus, adeno-associated virus, and retrovirus vectors, in addition to other particles that introduce DNA into cells, such as liposomes. Additionally, techniques such as those described above may be used for the introduction of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene sequences into human cells.

Cells, preferably, autologous cells, containing 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 expressing gene sequences may then be introduced or reintroduced into the subject at positions which allow for the amelioration of at least one symptom of a cancer. Such cell replacement techniques may be preferred, for example, when the gene product is a secreted, extracellular gene product.

Pharmaceutical Compositions

Another aspect of the invention pertains to methods for treating a subject suffering from a disease. These methods involve administering to a subject an agent which modulates 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 expression or activity (e.g., an agent identified by a screening assay described herein), or a combination of such agents. In another embodiment, the method involves administering to a subject a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein or nucleic acid molecule as therapy to compensate for reduced, aberrant, or unwanted 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 expression or activity.

Stimulation of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity is desirable in situations in which 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 is abnormally downregulated and/or in which increased 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity is likely to have a beneficial effect. Likewise, inhibition of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity is desirable in situations in which 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 is abnormally upregulated and/or in which decreased 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity is likely to have a beneficial effect.

The agents which modulate 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity can be administered to a subject using pharmaceutical compositions suitable for such administration. Such compositions typically comprise the agent (e.g., nucleic acid molecule, protein, or antibody) and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition used in the therapeutic methods of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the agent that modulates 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity (e.g., a fragment of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein or an anti-15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The agents that modulate 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity can also be prepared in the form of suppositories (e.g., with conventional suppository bases such as cocoa butter and other glycerides) or retention enemas for rectal delivery.

In one embodiment, the agents that modulate 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the agent that modulates 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity and the particular therapeutic effect to be achieved, and the limitations inherent in the art of compounding such an agent for the treatment of subjects.

Toxicity and therapeutic efficacy of such agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and can be expressed as the ratio LD50/ED50. Agents which exhibit large therapeutic indices are preferred. While agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage for use in humans. The dosage of such 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 modulating agents lies preferably within a range of circulating concentrations that include the ED50 with little or no toxicity. The dosage may vary within this range depending upon the dosage form employed and the route of administration utilized. For any agent used in the therapeutic methods of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e, including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram).

It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2^{nd} Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

The nucleic acid molecules used in the methods of the invention can be inserted into vectors and used as gene therapy vectors. Gene therapy vectors can be delivered to a subject by, for example, intravenous injection, local administration (see U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

Pharmacogenomics

In conjunction with the therapeutic methods of the invention, pharmacogenomics (i.e., the study of the relationship between a subject's genotype and that subject's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer an agent which modulates 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity, as well as tailoring the dosage and/or therapeutic regimen of treatment with an agent which modulates 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity.

Pharmacogenomics deals with clinically significant hereditary variations in the response to drugs due to altered drug disposition and abnormal action in affected persons. See, for example, Eichelbaum, M. et al. (1996) Clin. Exp.Pharmacol. Physiol. 23(10-11): 983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate aminopeptidase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants). Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach” can be utilized to identify genes that predict drug response. According to this method, if a gene that encodes a drug target is known (e.g., a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein used in the methods of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and the cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling” can be utilized to identify genes that predict drug response. For example, the gene expression of an animal dosed with a drug (e.g., a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 molecule or 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 modulator used in the methods of the present invention) can give an indication whether gene pathways related to toxicity have been turned on.

Information generated from more than one of the above pharmacogenomics approaches can be used to determine appropriate dosage and treatment regimens for prophylactic or therapeutic treatment of a subject. This knowledge, when applied to dosing or drug selection, can avoid adverse reactions or therapeutic failure and, thus, enhance therapeutic or prophylactic efficiency when treating a subject suffering from a cancer, with an agent which modulates 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity.

Recombinant Expression Vectors and Host Cells Used in the Methods of the Invention

The methods of the invention (e.g., the screening assays described herein) include the use of vectors, preferably expression vectors, containing a nucleic acid encoding a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors to be used in the methods of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel (1990) Methods Enzymol. 185:3-7. Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 proteins, mutant forms of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 proteins, fusion proteins, and the like).

The recombinant expression vectors to be used in the methods of the invention can be designed for expression of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 proteins in prokaryotic or eukaryotic cells. For example, 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 proteins can be expressed in bacterial cells such as E. coli, insect cells (using baculovirus expression vectors), yeast cells, or mammalian cells. Suitable host cells are discussed further in Goeddel (1990) supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity assays, (e.g., direct assays or competitive assays described in detail below), or to generate antibodies specific for 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 proteins. In a preferred embodiment, a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 fusion protein expressed in a retroviral expression vector of the present invention can be utilized to infect bone marrow cells which are subsequently transplanted into irradiated recipients. The pathology of the subject recipient is then examined after sufficient time has passed (e.g., six weeks).

In another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J. et al., Molecular Cloning: A Laboratory Manual. 2^{nd} ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid).

The methods of the invention may further use a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific, or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes, see Weintraub, H. et al., Antisense RNA as a molecular tool for genetic analysis, Reviews—Trends in Genetics, Vol. 1(1) 1986.

Another aspect of the invention pertains to the use of host cells into which a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 nucleic acid molecule of the invention is introduced, e.g., a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 nucleic acid molecule within a recombinant expression vector or a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook et al. (Molecular Cloning: A Laboratory Manual. 2^{nd}, ed., Cold Spring Harbor Laboratory, Cold Spring Harbor Laboratory Press, Cold Spring Harbor, N.Y., 1989), and other laboratory manuals.

A host cell used in the methods of the invention, such as a prokaryotic or eukaryotic host cell in culture, can be used to produce (i.e., express) a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein. Accordingly, the invention further provides methods for producing a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein using the host cells of the invention. In one embodiment, the method comprises culturing the host cell of the invention (into which a recombinant expression vector encoding a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein has been introduced) in a suitable medium such that a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein is produced. In another embodiment, the method further comprises isolating a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein from the medium or the host cell.

Isolated Nucleic Acid Molecules Used in the Methods of the Invention

The methods of the invention include the use of isolated nucleic acid molecules that encode 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 proteins or biologically active portions thereof, as well as nucleic acid fragments sufficient for use as hybridization probes to identify 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249-encoding nucleic acid molecules (e.g., 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 mRNA) and fragments for use as PCR primers for the amplification or mutation of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

A nucleic acid molecule used in the methods of the present invention, e.g., a nucleic acid molecule having the nucleotide sequence of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141 or 143, or a portion thereof, can be isolated using standard molecular biology techniques and the sequence information provided herein. Using all or portion of the nucleic acid sequence of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141 or 143, as a hybridization probe, 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 nucleic acid molecules can be isolated using standard hybridization and cloning techniques (e.g., as described in Sambrook, J., Fritsh, E. F., and Maniatis, T. Molecular Cloning: A Laboratory Manual. 2^{nd} ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141 or 143 can be isolated by the polymerase chain reaction (PCR) using synthetic oligonucleotide primers designed based upon the sequence of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141 or 143.

A nucleic acid used in the methods of the invention can be amplified using cDNA, mRNA or, alternatively, genomic DNA as a template and appropriate oligonucleotide primers according to standard PCR amplification techniques. Furthermore, oligonucleotides corresponding to 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 nucleotide sequences can be prepared by standard synthetic techniques, e.g., using an automated DNA synthesizer.

In a preferred embodiment, the isolated nucleic acid molecules used in the methods of the invention comprise the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141 or 143, a complement of the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141 or 143, or a portion of any of these nucleotide sequences. A nucleic acid molecule which is complementary to the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141 or 143, is one which is sufficiently complementary to the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141 or 143, such that it can hybridize to the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141 or 143, thereby forming a stable duplex.

In still another preferred embodiment, an isolated nucleic acid molecule used in the methods of the present invention comprises a nucleotide sequence which is at least about 55%, 60%, 65%, 70%, 75%, 80%, 85%, 90%, 95%, 96%, 97%, 98%, 99% or more identical to the entire length of the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141 or 143, or a portion of any of this nucleotide sequence.

Moreover, the nucleic acid molecules used in the methods of the invention can comprise only a portion of the nucleic acid sequence of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141 or 143 121, 123, 125, 127, 129, 131, 133, 135, 137, 139, 141 or 143, for example, a fragment which can be used as a probe or primer or a fragment encoding a portion of a 15986, 2188, 20743, 9148, 9151, 9791, 44252, 14184, 42461, 8204, 7970, 25552, 21657, 26492, 2411, 15088; 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein, e.g., a biologically active portion of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein. The probe/primer typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141 or 143, of an anti-sense sequence of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141 or 143, or of a naturally occurring allelic variant or mutant of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141 or 143. In one embodiment, a nucleic acid molecule used in the methods of the present invention comprises a nucleotide sequence which is greater than 100, 100-200, 200-300, 300-400, 400-500, 500-600, 600-700, 700-800, 800-900, 900-1000, 1000-1100, 1100-1200, 1200-1300, or more nucleotides in length and hybridizes under stringent hybridization conditions to a nucleic acid molecule of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141 or 143.

As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4 and 6. Additional stringent conditions can be found in Molecular Cloning: A Laboratory Manual, Sambrook et al., Cold Spring Harbor Press, Cold Spring Harbor, N.Y. (1989), chapters 7, 9 and 11. A preferred, non-limiting example of stringent hybridization conditions includes hybridization in 4× sodium chloride/sodium citrate (SSC), at about 65-70° C. (or hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41(% G+C)−(600/N), where N is the number of bases in the hybrid, and [Na+] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C., see e.g., Church and Gilbert (1984) Proc. Natl. Acad. Sci. USA 81:1991-1995, (or alternatively 0.2×SSC, 1% SDS).

In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein, such as by measuring a level of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249-encoding nucleic acid in a sample of cells from a subject e.g., detecting 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 mRNA levels or determining whether a genomic 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene has been mutated or deleted.

The methods of the invention further encompass the use of nucleic acid molecules that differ from the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141 or 143, due to degeneracy of the genetic code and thus encode the same 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 proteins as those encoded by the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141 or 143. In another embodiment, an isolated nucleic acid molecule included in the methods of the invention has a nucleotide sequence encoding a protein having an amino acid sequence shown in SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142 or 144.

The methods of the invention further include the use of allelic variants of human 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249, e.g., functional and non-functional allelic variants. Functional allelic variants are naturally occurring amino acid sequence variants of the human 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein that maintain a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity. Functional allelic variants will typically contain only conservative substitution of one or more amino acids of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142 or 144, or substitution, deletion or insertion of non-critical residues in non-critical regions of the protein.

Non-functional allelic variants are naturally occurring amino acid sequence variants of the human 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein that do not have a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity. Non-functional allelic variants will typically contain a non-conservative substitution, deletion, or insertion or premature truncation of the amino acid sequence of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142 or 144 or a substitution, insertion or deletion in critical residues or critical regions of the protein.

The methods of the present invention may further use non-human orthologues of the human 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein. Orthologues of the human 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein are proteins that are isolated from non-human organisms and possess the same 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity.

The methods of the present invention further include the use of nucleic acid molecules comprising the nucleotide sequence of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141 or 143 or a portion thereof, in which a mutation has been introduced. The mutation may lead to amino acid substitutions at “non-essential” amino acid residues or at “essential” amino acid residues. A “non-essential” amino acid residue is a residue that can be altered from the wild-type sequence of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 (e.g., the sequence of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142 or 144) without altering the biological activity, whereas an “essential” amino acid residue is required for biological activity. For example, amino acid residues that are conserved among the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 proteins of the present invention are not likely to be amenable to alteration.

Mutations can be introduced into SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141 or 143, by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein is preferably replaced with another amino acid residue from the same side chain family. Alternatively, in another embodiment, mutations can be introduced randomly along all or part of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 coding sequence, such as by saturation mutagenesis, and the resultant mutants can be screened for 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 biological activity to identify mutants that retain activity. Following mutagenesis of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141 or 143, the encoded protein can be expressed recombinantly and the activity of the protein can be determined using the assay described herein.

Another aspect of the invention pertains to the use of isolated nucleic acid molecules which are antisense to the nucleotide sequence of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141 or 143. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 coding strand, or to only a portion thereof. In one embodiment, an antisense nucleic acid molecule is antisense to a “coding region” of the coding strand of a nucleotide sequence encoding a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249. The term “noncoding region” refers to 5′ and 3′ sequences which flank the coding region that are not translated into amino acids (also referred to as 5′ and 3′ untranslated regions).

Given the coding strand sequences encoding 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 disclosed herein, antisense nucleic acids of the invention can be designed according to the rules of Watson and Crick base pairing. The antisense nucleic acid molecule can be complementary to the entire coding region of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 mRNA, but more preferably is an oligonucleotide which is antisense to only a portion of the coding or noncoding region of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 mRNA. For example, the antisense oligonucleotide can be complementary to the region surrounding the translation start site of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest). Antisense nucleic acid molecules used in the methods of the invention are further described above.

In yet another embodiment, the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 nucleic acid molecules used in the methods of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. (1996) Proc. Natl. Acad. Sci. 93:14670-675.

PNAs of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 nucleic acid molecules can be used in the therapeutic and diagnostic applications described herein. For example, PNAs can be used as antisense or antigene agents for sequence-specific modulation of gene expression by, for example, inducing transcription or translation arrest or inhibiting replication. PNAs of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. et al. (1996) supra)); or as probes or primers for DNA sequencing or hybridization (Hyrup B. et al. (1996) supra; Perry-O'Keefe et al. (1996) supra).

In another embodiment, PNAs of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. et al. (1996) supra). The synthesis of PNA-DNA chimeras can be performed as described in Hyrup B. et al. (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide used in the methods of the invention may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO88/09810) or the blood-brain barrier (see, e.g., PCT Publication No. WO89/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

Isolated 15986, 2188, 20743, 9148, 9151, 9791, 44252, 14184, 42461, 8204, 7970, 25552, 21657, 26492, 2411, 15088, 1905.28899, 63380, 33935, 10480, 12686, 25501, 17694, 15701.53062, 49908, 21612, 38949.6216, 46863, 9235, 2201, 6985.9883, 12238, 18057, 21617, 39228, 49928, 54476, 62113, 64316.12264, 32362, 58198, 2887, 3205, 8557, 9600, 9693, 44867, 53058, 55556, 57658.2208, 10252, 10302, 14218, 33877, 10317, 10485, 25964, 14815, 1363, 1397, 14827, 21708, 3801, 64698, 2179 or 13249 Proteins and Anti-15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 Antibodies Used in the

Methods of the Invention

The methods of the invention include the use of isolated 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 proteins, and biologically active portions thereof, as well as polypeptide fragments suitable for use as immunogens to raise anti-15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 antibodies. In one embodiment, native 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 proteins can be isolated from cells or tissue sources by an appropriate purification scheme using standard protein purification techniques. In another embodiment, 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 proteins are produced by recombinant DNA techniques. Alternative to recombinant expression, a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein or polypeptide can be synthesized chemically using standard peptide synthesis techniques.

As used herein, a “biologically active portion” of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein includes a fragment of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein having a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity. Biologically active portions of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein include peptides comprising amino acid sequences sufficiently identical to or derived from the amino acid sequence of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein, e.g., the amino acid sequence shown in SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142 or 144, which include fewer amino acids than the full length 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 proteins, and exhibit at least one activity of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein. Typically, biologically active portions comprise a domain or motif with at least one activity of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein (e.g., the N-terminal region of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein that is believed to be involved in the regulation of apoptotic activity). A biologically active portion of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein can be a polypeptide which is, for example, 25, 50, 75, 100, 125, 150, 175, 200, 250, 300 or more amino acids in length. Biologically active portions of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein can be used as targets for developing agents which modulate a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 activity.

In a preferred embodiment, the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein used in the methods of the invention has an amino acid sequence shown in SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142 or 144. In other embodiments, the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein is substantially identical to SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142 or 144, and retains the functional activity of the protein of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142 or 144, yet differs in amino acid sequence due to natural allelic variation or mutagenesis, as described in detail in subsection V above. Accordingly, in another embodiment, the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein used in the methods of the invention is a protein which comprises an amino acid sequence at least about 50%, 55%, 60%, 65%, 70%, 75%, 80%, 85%, 90%, 95%, 96%, 97%, 98%, 99% or more identical to SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142 or 144.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 amino acid sequence of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142 or 144 having 500 amino acid residues, at least 75, preferably at least 150, more preferably at least 225, even more preferably at least 300, and even more preferably at least 400 or more amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. 48:444-453 (1970)) algorithm which has been incorporated into the GAP program in the GCG software package, using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (Comput. Appl. Biosci. 4:11-17 (1988)) which has been incorporated into the ALIGN program (version 2.0 or 2.0U), using a PAM120 weight residue table, a gap length penalty of 12 and a gap penalty of 4.

The methods of the invention may also use 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 chimeric or fusion proteins. As used herein, a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 “chimeric protein” or “fusion protein” comprises a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 polypeptide operatively linked to a non-15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 polypeptide. An “15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 polypeptide” refers to a polypeptide having an amino acid sequence corresponding to a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 molecule, whereas a “non-15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 polypeptide” refers to a polypeptide having an amino acid sequence corresponding to a protein which is not substantially homologous to the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein, e.g., a protein which is different from the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein and which is derived from the same or a different organism. Within a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 fusion protein the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 polypeptide can correspond to all or a portion of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein. In a preferred embodiment, a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 fusion protein comprises at least one biologically active portion of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein. In another preferred embodiment, a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 fusion protein comprises at least two biologically active portions of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein. Within the fusion protein, the term “operatively linked” is intended to indicate that the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 polypeptide and the non-15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 polypeptide are fused in-frame to each other. The non-15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 polypeptide can be fused to the N-terminus or C-terminus of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 polypeptide.

For example, in one embodiment, the fusion protein is a GST-15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 fusion protein in which the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 sequences are fused to the C-terminus of the GST sequences. Such fusion proteins can facilitate the purification of recombinant 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249.

In another embodiment, this fusion protein is a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein containing a heterologous signal sequence at its N-terminus. In certain host cells (e.g., mammalian host cells), expression and/or secretion of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 can be increased through use of a heterologous signal sequence.

The 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 fusion proteins used in the methods of the invention can be incorporated into pharmaceutical compositions and administered to a subject in vivo. The 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 fusion proteins can be used to affect the bioavailability of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 substrate. Use of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 fusion proteins may be useful therapeutically for the treatment of disorders caused by, for example, (i) aberrant modification or mutation of a gene encoding a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein; (ii) mis-regulation of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 gene; and (iii) aberrant post-translational modification of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein.

Moreover, the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249-fusion proteins used in the methods of the invention can be used as immunogens to produce anti-15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 antibodies in a subject, to purify 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 ligands and in screening assays to identify molecules which inhibit the interaction of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 with a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 substrate.

Preferably, a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 chimeric or fusion protein used in the methods of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining; and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249-encoding nucleic acid can be cloned into such an expression vector such that the fusion moiety is linked in-frame to the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein.

The present invention also pertains to the use of variants of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 proteins which function as either 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 agonists (mimetics) or as 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 antagonists. Variants of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 proteins can be generated by mutagenesis, e.g., discrete point mutation or truncation of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein. An agonist of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 proteins can retain substantially the same, or a subset, of the biological activities of the naturally occurring form of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein. An antagonist of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein can inhibit one or more of the activities of the naturally occurring form of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein by, for example, competitively modulating a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249-mediated activity of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein.

In one embodiment, variants of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein which function as either 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 agonists (mimetics) or as 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 antagonists can be identified by screening combinatorial libraries of mutants, e.g., truncation mutants, of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein for 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein agonist or antagonist activity. In one embodiment, a variegated library of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 variants is generated by combinatorial mutagenesis at the nucleic acid level and is encoded by a variegated gene library. A variegated library of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 variants can be produced by, for example, enzymatically ligating a mixture of synthetic oligonucleotides into gene sequences such that a degenerate set of potential 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 sequences is expressible as individual polypeptides, or alternatively, as a set of larger fusion proteins (e.g., for phage display) containing the set of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 sequences therein. There are a variety of methods which can be used to produce libraries of potential 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In addition, libraries of fragments of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein coding sequence can be used to generate a variegated population of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 fragments for screening and subsequent selection of variants of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein. In one embodiment, a library of coding sequence fragments can be generated by treating a double stranded PCR fragment of a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 variants (Arkin and Yourvan (1992) Proc. Natl. Acad. Sci. USA 89:7811-7815; Delgrave et al. (1993) Protein Engineering 6(3):327-331).

The methods of the present invention further include the use of anti-15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 antibodies. An isolated 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein, or a portion or fragment thereof, can be used as an immunogen to generate antibodies that bind 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 using standard techniques for polyclonal and monoclonal antibody preparation. A full-length 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein can be used or, alternatively, antigenic peptide fragments of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 can be used as immunogens. The antigenic peptide of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 comprises at least 8 amino acid residues of the amino acid sequence shown in SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142 or 144 and encompasses an epitope of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 such that an antibody raised against the peptide forms a specific immune complex with the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 that are located on the surface of the protein, e.g., hydrophilic regions, as well as regions with high antigenicity.

A 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 immunogen is typically used to prepare antibodies by immunizing a suitable subject, (e.g., rabbit, goat, mouse, or other mammal) with the immunogen. An appropriate immunogenic preparation can contain, for example, recombinantly expressed 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein or a chemically synthesized 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 polypeptide. The preparation can further include an adjuvant, such as Freund's complete or incomplete adjuvant, or similar immunostimulatory agent. Immunization of a suitable subject with an immunogenic 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 preparation induces a polyclonal anti-15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 antibody response.

The term “antibody” as used herein refers to immunoglobulin molecules and immunologically active portions of immunoglobulin molecules, i.e., molecules that contain an antigen binding site which specifically binds (immunoreacts with) an antigen, such as a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 molecules. The term “monoclonal antibody” or “monoclonal antibody composition”, as used herein, refers to a population of antibody molecules that contain only one species of an antigen binding site capable of immunoreacting with a particular epitope of 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249. A monoclonal antibody composition thus typically displays a single binding affinity for a particular 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein with which it immunoreacts.

Polyclonal anti-15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 antibodies can be prepared as described above by immunizing a suitable subject with a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 immunogen. The anti-15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 antibody titer in the immunized subject can be monitored over time by standard techniques, such as with an enzyme linked immunosorbent assay (ELISA) using immobilized 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249. If desired, the antibody molecules directed against 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 can be isolated from the mammal (e.g., from the blood) and further purified by well known techniques, such as protein A chromatography to obtain the IgG fraction. At an appropriate time after immunization, e.g., when the anti-15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985) Monoclonal Antibodies and Cancer Therapy, Alan R. Liss, Inc., pp. 77-96) or trioma techniques. The technology for producing monoclonal antibody hybridomas is well known (see generally Kenneth, R. H. in Monoclonal Antibodies: A New Dimension In Biological Analyses, Plenum Publishing Corp., New York, N.Y. (1980); Lerner, E. A. (1981) Yale J. Biol. Med. 54:387-402; Gefter, M. L. et al. (1977) Somatic Cell Genet. 3:231-36). Briefly, an immortal cell line (typically a myeloma) is fused to lymphocytes (typically splenocytes) from a mammal immunized with a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 immunogen as described above, and the culture supernatants of the resulting hybridoma cells are screened to identify a hybridoma producing a monoclonal antibody that binds 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249.

Any of the many well known protocols used for fusing lymphocytes and immortalized cell lines can be applied for the purpose of generating an anti-15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 monoclonal antibody (see, e.g., G. Galfre et al. (1977) Nature 266:55052; Gefter et al. (1977) supra; Lerner (1981) supra; and Kenneth (1980) supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249, e.g., using a standard ELISA assay.

Alternative to preparing monoclonal antibody-secreting hybridomas, a monoclonal anti-15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 antibody can be identified and isolated by screening a recombinant combinatorial immunoglobulin library (e.g., an antibody phage display library) with 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 to thereby isolate immunoglobulin library members that bind 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrad et al. (1991) Bio/Technology 9:1373-1377; Hoogenboom et al. (1991) Nuc. Acid Res. 19:4133-4137; Barbas et al. (1991) Proc. Natl. Acad. Sci. USA 88:7978-7982; and McCafferty et al. (1990) Nature 348:552-554.

Additionally, recombinant anti-15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 antibodies, such as chimeric and humanized monoclonal antibodies, comprising both human and non-human portions, which can be made using standard recombinant DNA techniques, are within the scope of the methods of the invention. Such chimeric and humanized monoclonal antibodies can be produced by recombinant DNA techniques known in the art, for example using methods described in Robinson et al. International Application No. PCT/US86/02269; Akira, et al. European Patent Application 184, 187; Taniguchi, M., European Patent Application 171, 496; Morrison et al. European Patent Application 173, 494; Neuberger et al. PCT International Publication No. WO 86/01533; Cabilly et al. U.S. Pat. No. 4,816,567; Cabilly et al. European Patent Application 125, 023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; Shaw et al. (1988) J. Natl. Cancer Inst. 80:1553-1559; Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

An anti-15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 antibody can be used to detect 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein (e.g., in a cellular lysate or cell supernatant) in order to evaluate the abundance and pattern of expression of the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 protein. Anti-15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

This invention is further illustrated by the following examples which should not be construed as limiting. The contents of all references, patents and published patent applications cited throughout this application, as well as the Figure and the Sequence Listing is incorporated herein by reference.

### EXAMPLES

### Example 1

### Tissue Distribution of Using Taqman™ Analysis

This example describes the TaqMan™ procedure. The Taqman™ procedure is a quantitative, reverse transcription PCR-based approach for detecting mRNA. The RT-PCR reaction exploits the 5′ nuclease activity of AmpliTaq Gold™ DNA Polymerase to cleave a TaqMan™ probe during PCR. Briefly, cDNA was generated from the samples of interest, e.g., heart, kidney, liver, skeletal muscle, and various vessels, and used as the starting material for PCR amplification. In addition to the 5′ and 3′ gene-specific primers, a gene-specific oligonucleotide probe (complementary to the region being amplified) was included in the reaction (i.e., the Taqman™ probe). The TaqMan™ probe includes the oligonucleotide with a fluorescent reporter dye covalently linked to the 5′ end of the probe (such as FAM (6-carboxyfluorescein), TET (6-carboxy-4,7,2′,7′-tetrachlorofluorescein), JOE (6-carboxy-4,5-dichloro-2,7-dimethoxyfluorescein), or VIC) and a quencher dye (TAMRA (6-carboxy-N,N,N′,N′-tetramethylrhodamine) at the 3′ end of the probe.

During the PCR reaction, cleavage of the probe separates the reporter dye and the quencher dye, resulting in increased fluorescence of the reporter. Accumulation of PCR products is detected directly by monitoring the increase in fluorescence of the reporter dye. When the probe is intact, the proximity of the reporter dye to the quencher dye results in suppression of the reporter fluorescence. During PCR, if the target of interest is present, the probe specifically anneals between the forward and reverse primer sites. The 5′-3′ nucleolytic activity of the AmpliTaq™ Gold DNA Polymerase cleaves the probe between the reporter and the quencher only if the probe hybridizes to the target. The probe fragments are then displaced from the target, and polymerization of the strand continues. The 3′ end of the probe is blocked to prevent extension of the probe during PCR. This process occurs in every cycle and does not interfere with the exponential accumulation of product. RNA was prepared using the trizol method and treated with DNase to remove contaminating genomic DNA. cDNA was synthesized using standard techniques. Mock cDNA synthesis in the absence of reverse transcriptase resulted in samples with no detectable PCR amplification of the control gene confirms efficient removal of genomic DNA contamination.

### Example 2

### Tissue Distribution of Using in Situ Analysis

For in situ analysis, various tissues, e.g., tissues obtained from normal colon, breast, lung, and ovarian normal tissue, as well as colon, breast, lung, and ovarian tumors, colon metastatic to the liver, and angiogenic tissues were first frozen on dry ice. Ten-micrometer-thick sections of the tissues were post-fixed with 4% formaldehyde in DEPC treated 1× phosphate-buffered saline at room temperature for 10 minutes before being rinsed twice in DEPC 1× phosphate-buffered saline and once in 0.1 M triethanolamine-HCl (pH 8.0). Following incubation in 0.25% acetic anhydride-0.1 M triethanolamine-HCl for 10 minutes, sections were rinsed in DEPC 2×SSC (1×SSC is 0.15M NaCl plus 0.015M sodium citrate). Tissue was then dehydrated through a series of ethanol washes, incubated in 100% chloroform for 5 minutes, and then rinsed in 100% ethanol for 1 minute and 95% ethanol for 1 minute and allowed to air dry.

Hybridizations were performed with ³⁵S-radiolabeled (5×10⁷ cpm/ml) cRNA probes. Probes were incubated in the presence of a solution containing 600 mM NaCl, 10 mM Tris (pH 7.5), 1 mM EDTA, 0.01% sheared salmon sperm DNA, 0.01% yeast tRNA, 0.05% yeast total RNA type X1, 1× Denhardt's solution, 50% formamide, 10% dextran sulfate, 100 mM dithiothreitol, 0.1% sodium dodecyl sulfate (SDS), and 0.1% sodium thiosulfate for 18 hours at 55° C.

After hybridization, slides were washed with 2×SSC. Sections were then sequentially incubated at 37° C. in TNE (a solution containing 10 mM Tris-HCl (pH 7.6), 500 mM NaCl, and 1 mM EDTA), for 10 minutes, in TNE with 10% g of RNase A per ml for 30 minutes, and finally in TNE for 10 minutes. Slides were then rinsed with 2×SSC at room temperature, washed with 2×SSC at 50° C. for 1 hour, washed with 0.2×SSC at 55° C. for 1 hour, and 0.2×SSC at 60° C. for 1 hour. Sections were then dehydrated rapidly through serial ethanol-0.3 M sodium acetate concentrations before being air dried and exposed to Kodak Biomax MR scientific imaging film for 24 hours and subsequently dipped in NB-2 photoemulsion and exposed at 4° C. for 7 days before being developed and counter stained.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed:

1. A method for identifying a compound capable of treating cancer or an angiogenic disorder, comprising:
a) combining a compound to be tested with a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 polypeptide under conditions suitable for binding of the test compound to the polypeptide; and
b) detecting binding of the test compound to the polypeptide to thereby identify a compound which binds to the polypeptide, thereby identifying a compound capable of treating cancer or an angiogenic disorder.

2. The method of claim 1, wherein the compound is selected from the group consisting of a small molecule, a peptide or an antibody.

3. The method of claim 1, wherein the polypeptide further comprises heterologous sequences.

4. The method of claim 1, wherein the polypeptide is an isolated polypeptide, a membrane-bound form of an isolated polypeptide or a cell comprising the polypeptide.

5. The method of claim 4, wherein the cell is a cancer cell.

6. The method of claim 1, wherein the cancer or an angiogenic disorder is a cancer of the lung, colon, breast, ovary or prostate.

7. The method of claim 1, wherein the binding of the test compound to the polypeptide is detected by a method selected from the group consisting of:
a) a competition binding assay;
b) an immunoassay; and
c) a yeast two-hybrid assay.

8. A method for identifying a compound capable of treating cancer or an angiogenic disorder, comprising:
a) combining a compound to be tested with a host cell expressing a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 polypeptide under conditions suitable for binding of the test compound to the polypeptide; and
b) detecting binding of the test compound to the polypeptide to thereby identify a compound which binds to the polypeptide, thereby identifying a compound capable of treating cancer or an angiogenic disorder.

9. The method of claim 8, wherein the compound is selected for the group consisting of a small molecule, a peptide, an antibody or an antisense nucleic acid molecule.

10. The method of claim 8, wherein the polypeptide further comprises heterologous sequences.

11. The method of claim 8, wherein the host cell is a cancer cell.

12. The method of claim 8, wherein the cancer or an angiogenic disorder is a cancer of the lung, colon, breast, ovary or prostate.

13. The method of claim 8, wherein the binding of the test compound to the polypeptide is detected by a method selected from the group consisting of:
a) a competition binding assay;
b) an immunoassay; and
c) a yeast two-hybrid assay.

14. A method of identifying a subject having a cancer, or at risk for developing a cancer comprising:
a) contacting a sample obtained from the subject comprising polypeptides with a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 binding substance; and
b) detecting the presence of a polypeptide in the sample that binds to the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 binding substance, thereby identifying a subject having a cancer, or at risk for developing a cancer.

15. The method of claim 14, wherein the binding substance is an antibody.

16. The method of claim 14, wherein the binding substance is detectably labeled.

17. A method for treating a subject having a cancer or a cancer characterized by aberrant 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 polypeptide activity or aberrant 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 nucleic acid expression comprising administering to the subject a 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 modulator, thereby treating said subject having a cancer.

18. The method of claim 17, wherein the disorder is a disorder associated with a cancer including but not limited to cancers of the lung, colon, breast, ovary or prostate or an angiogenic disorder.

19. The method of claim 17, wherein the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 modulator is administered in a pharmaceutically acceptable formulation.

20. The method of 17, wherein the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 modulator is capable of modulating 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 polypeptide activity.

21. The method of claim 20, wherein the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 modulator is an anti-15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 antibody.

22. The method of claim 17, wherein the 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 modulator is capable of modulating 15986, 2188, 20743, 9148, 9151, 9791, 44252, 14184, 42461, 8204, 7970, 25552, 21657, 26492, 2411, 15088, 1905, 28899, 63380, 33935, 10480, 12686, 25501, 17694, 15701, 53062, 49908, 21612, 38949, 6216, 46863, 9235, 2201, 6985, 9883, 12238, 18057, 21617, 39228, 49928, 54476, 62113, 64316, 12264, 32362, 58198, 2887, 3205, 8557, 9600, 9693, 44867, 53058, 55556, 57658, 2208, 10252, 10302, 14218, 33877, 10317, 10485, 25964, 14815, 1363, 1397, 14827, 21708, 3801, 64698, 2179 or 13249 nucleic acid expression.
